(19) United States
(12) Patent Application Publication
(10) Pub. No.: US 2002/0168663 A1
(43) Pub. Date: Nov. 14, 2002

(54) Methods for DNA conjugation onto solid phase including related optical biodiscs and disc drive systems

(76) Inventors: Brigitte Chau Phan, Irvine, CA (US); Jorma Antero Virtanen, Irvine, CA (US); Amethyst Hoang Lam, Irvine, CA (US); KaYuen Yeung, San Francisco, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/086,941

(22) Filed: Feb. 26, 2002

Related U.S. Application Data
(63) Continuation-in-part of application No. 10/038,297, filed on Jan. 4, 2002.
(60) Provisional application No. 60/271,922, filed on Feb. 27, 2001.
(60) Provisional application No. 60/272,485, filed on Mar. 1, 2001.
(60) Provisional application No. 60/275,643, filed on Mar. 14, 2001.
(60) Provisional application No. 60/277,854, filed on Mar. 22, 2001.
(60) Provisional application No. 60/278,685, filed on Mar. 26, 2001.
(60) Provisional application No. 60/314,906, filed on Aug. 24, 2001.
(60) Provisional application No. 60/352,270, filed on Jan. 30, 2002.

(51) Int. Cl. C12Q 1/68; G03C 1/492; G03C 1/494; G03C 1/76; C12M 1/34

(52) U.S. Cl. 435/6; 435/287.2; 430/273.1

(57) ABSTRACT

The invention provides for methods of conjugating biochemical probes onto a solid phase for use in biomedical assays as implemented in conjunction with an optical bio-disc system. The method includes determining the suitability of a test solid phase for purposes of use in a dual bead assay, selecting a test solid phase, conjugating a probe to the test solid phase in the presence or absence of a cross-linking agent, and determining the total amount of probe bound to the test solid phase in the presence or absence of a cross-linking agent. The method is further employed to determine the percentage of probe bound covalently or non-covalently to the solid phase and calculating the percentage of probe bound covalently thereby selecting the solid phase with the highest conjugation efficiency. The invention is further directed at methods for determining whether a target agent is present in a biological sample. A bio-disc for performing a dual bead assay according to these methods is also provided.

### CROSS-REFERENCE TO RELATED APPLICATIONS

This application is a continuation-in-part of U.S. application Ser. No. 10/038,297 filed Jan. 4, 2002 which claimed the benefit of priority from U.S. Provisional Application Serial No. 60/259,806 filed Jan. 4, 2001 and U.S. Provisional Application Serial No. 60/271,922 filed Feb. 27, 2001.

This application also claims the benefit of priority from U.S. Provisional Application Serial No. 60/271,922 filed the Feb. 27, 2001; U.S. Provisional Application Serial No. 60/272,485 filed Mar. 1, 2001; U.S. Provisional Application Serial No. 60/275,643 filed Mar. 14, 2001; U.S. Provisional Application Serial No. 60/277,854 filed Mar. 22, 2001; U.S. Provisional Application Serial No. 60/278,685 filed Mar. 26, 2001; U.S. Provisional Application Serial No. 60/314,906 filed Aug. 24, 2001; and U.S. Provisional Application Serial No. 60/352,270 filed Jan. 30, 2002.

Each of the above applications is herein incorporated by reference in its entirety.

### BACKGROUND OF THE INVENTION

1. Field of the Invention

The present invention relates to optical analysis systems for performing assays. The invention further relates to methods for DNA conjugation onto solid phase including related optical bio-discs and disc drive systems. The invention is further directed to dual bead assays performed on optical bio-discs.

2. Discussion of the Related Art

There is a significant need to make diagnostic assays and forensic assays of all types faster and more local to the end-user. Ideally, clinicians, patients, investigators, the military, other health care personnel, and consumers should be able to test themselves for the presence of certain factors or indicators in their systems, and for the presence of certain biological material at a crime scene or on a battlefield. At present, there are a number of silicon-based chips with nucleic acids and/or proteins attached thereto, which are commercially available or under development. These chips are not for use by the end-user, or for use by persons or entities lacking very specialized expertise and expensive equipment.

### SUMMARY OF THE INVENTION

The present invention relates to performing assays, and particularly to using dual bead structures on a disc. The invention includes methods for preparing assays, methods for performing assays, discs for performing assays, and related detection systems.

In one aspect, the present invention includes methods for determining whether a target agent is present in a biological sample. These methods can include mixing capture beads, each having at least one transport probe, reporter beads, each having at least one signal probe, and a biological sample. These components are mixed under binding conditions that permit formation of a dual bead complex if the target agent is present in the sample. The dual bead complex thus includes a reporter bead and a capture bead each bound to the target agent. The dual bead complex is isolated from the mixture to obtain an isolate. The isolate is then exposed to a capture field on an optical disc. The capture field has a capture agent that binds specifically to the signal probe or transport probe of the dual bead complex. The dual bead complex in the optical disc is then detected to indicate that the target agent is present in the sample and, if desired, to indicate a concentration.

The capture beads can have a specified size and have a characteristic that makes them “isolatable.” The capture beads are preferably magnetic, in which case the isolating of dual bead complex (and some capture beads not part of a complex) in a mixture includes subjecting the mixture to a magnetic field with a permanent magnet or an electromagnet. Capture beads that are not magnetic may be isolated by centrifugal forces.

The reporter bead should have characteristics that make it identifiable and distinguishable with detection. The reporter beads can be made of one of a number of materials, such as latex, gold, plastic, steel, or titanium, and should have a known and specified size. The reporter beads can be fluorescent and can be yellow, green, red, or blue, for example.

The dual bead complex can be formed on the disc itself, or outside the disc and added to the disc. To form the dual bead complex off disc, methods referred to here as “single-step” or “two-step” can be employed. In the two-step method, the mixture initially includes capture beads and the sample. The capture beads are then isolated to wash away unbound sample and leave bound and unbound capture beads in a first isolate. Reporter beads are then added to the first isolate to produce dual bead complex structures and the isolation process is repeated. The resulting isolate leaves dual bead complex with reporters, but also includes unbound capture beads without reporters. The reporters make the dual bead complex detectable.

In the “single-step” method, the capture beads, reporter beads, and sample are mixed together from the start and then the isolation process isolates dual bead complex along with unbound capture beads.

These methods for producing and isolating dual bead complex structures can be performed on the disc. The sample and beads can be added to the disc together, or the beads can be pre-loaded on the disc so that only a sample needs to be added. The sample and beads can be added in a mixing chamber on the disc, and the disc can be rotated in one direction or in both to assist the mixing. An isolate can then be created, such as by applying an electromagnet and rotating to cause the material other than the capture beads to be moved to a waste chamber. The isolate is then directed through rotation to capture fields.

The dual bead complex structures can be detected on the capture field by use of various methods. In one embodiment, the detecting includes directing a beam of electromagnetic energy from a disc drive toward the capture field and analyzing electromagnetic energy returned from or transmitted past the reporter bead of the dual bead complex attached to the capture field. The disc drive assembly can include a detector and circuitry or software that senses the detector signal for a sufficient transition between light and dark (referred to as an “event”) to spot a reporter bead.

Beads can, alternatively, be detected based on their fluorescence. In this case, the energy source in the disc drive preferably has a wavelength controllable light source and a detector that is or can be made specific to a particular wavelength. Alternatively, a disc drive can be made with a specific light source and detector to produce a dedicated device, in which case the source may only need fine-tuning.

The biological sample can include blood, serum, plasma, cerebrospinal fluid, breast aspirate, synovial fluid, pleural fluid, perintoneal fluid, pericardial fluid, urine, saliva, amniotic fluid, semen, mucus, a hair, feces, a biological particulate suspension, a single-stranded or double-stranded nucleic acid molecule, a cell, an organ, a tissue, or a tissue extract, or any other sample that includes a target that may be bound through chemical or biological processes. Further details relating to other aspects associated with the selection and detection of various targets is disclosed in, for example, commonly assigned and co-pending U.S. Provisional Patent Application Serial No. 60/278,697 entitled “Dual Bead Assays for Detecting Medical Targets” filed Mar. 26, 2001, which is incorporated herein by reference in its entirety.

In addition to these medical uses, the embodiments of the present invention can be used in other ways, such as for testing for impurities in a sample, such as food or water, or for otherwise detecting the presence of a material, such as a biological warfare agent.

The target agent can include, for example, a nucleic acid (such as DNA or RNA) or a protein (such as an antigen or an antibody). If a nucleic acid, both the transport probe and the signal probe can be a nucleic acid molecule complementary to the target nucleic acid. If a protein, both the transport probe and the signal probe can be an antibody that specifically binds the target protein.

The transport probe or signal probe can bind specifically to the capture agent on the optical disc due to a high affinity between the probe and the capture agent. This high affinity can, for example, be the result of a strong protein-protein affinity (i.e., antigen-antibody affinity), or the result of a complementarity between two nucleic acid molecules.

Preferably the binding is to the signal probe, and then the disc is rotated to move unbound structures, including capture beads not bound to reporter beads, away from the capture field. If the binding is to the transport probe, unbound capture beads will be included, although the reporter beads are still the beads that are detected. This may be acceptable if the detection is for producing a yes/no answer, or if a fine concentration detection is not otherwise required.

The transport probe and signal probe can each be one or more probes selected from the group consisting of single-stranded DNA, double-stranded DNA, single-stranded RNA, peptide nucleic acid, biotin, streptavidin, an antigen, an antibody, a receptor protein, and a ligand. In a further embodiment, each transport probe includes double-stranded DNA and single-stranded DNA, wherein the double-stranded DNA is proximate to the capture layer of the optical disc and the single-stranded DNA is distal relative to the capture layer of the optical disc.

The reporter bead and/or signal probe can be biotinylated and the capture agent can include streptavidin or neutravidin. Chemistry for affixing capture agents to the capture layer of the optical disc are generally known, especially in the case of affixing a protein or nucleic acid to solid surfaces. The capture agent can be affixed to the capture layer by use of an amino group or a thiol group.

The target agent can include a nucleic acid characteristic of a disease, or a nucleotide sequence specific for a person, or a nucleotide sequence specific for an organism, which may be a bacterium, a virus, a mycoplasm, a fungus, a plant, or an animal. The target agent can include a nucleic acid molecule associated with cancer in a human. The target nucleic acid molecule can include a nucleic acid, which is at least a portion of a gene selected from the group consisting of HER2neu, p52, p53, p21, and bcl-2. The target agent can be an antibody that is present only in a subject infected with HIV-1, a viral protein antigen, or a protein characteristic of a disease state in a subject. The methods and apparatus of the present invention can be used for determining whether a subject is infected by a virus, whether nucleic acid obtained from a subject exhibits a single nucleotide mutation (SNM) relative to corresponding wild-type nucleic acid sequence, or whether a subject expresses a protein of interest, such as a bacterial protein, a fungal protein, a viral protein, an HIV protein, a hepatitis C protein, a hepatitis B protein, or a protein known to be specifically associated with a disease. An example of a dual bead experiment detecting a nucleic acid target is presented below in Example 1.

According to another aspect of the invention, there is provided multiplexing methods wherein more than one target agent (e.g., tens, hundreds, or even thousands of different target agents) can be identified on one optical analysis disc. Multiple capture agents can be provided in a single chamber together in capture fields, or separately in separate capture fields. Different reporter beads can be used to be distinguishable from each other, such as beads that fluoresce at different wavelengths or different size reporter beads. Experiments were performed to identify two different targets using the multiplexing technique. An example of one such assay is discussed below in Example 2.

In accordance with yet another aspect, the invention includes an optical disc with a substrate, a capture layer associated with the substrate, and a capture agent bound to the capture layer, such that the capture agent binds to a dual bead complex. Multiple different capture agents can be used for different types of dual bead complexes. The disc can be designed to allow for some dual bead processing on the disc with appropriate chambers and fluidic structures, and can be pre-loaded with reporter and capture beads so that only a sample needs to be added to form the dual bead complex structures.

According to still a further aspect of this invention, there is provided a disc and disc drive system for performing dual bead assays. The disc drive can include an electromagnet for performing the isolation process, and may include appropriate light source control and detection for the type of reporter beads used. The disc drive can be optical or magneto-optical.

For processing performed on the disc, the drive may advantageously include an electromagnet, and the disc preferably has a mixing chamber, a waste chamber, and capture area. In this embodiment, the sample is mixed with beads in the mixing chamber, a magnetic field is applied adjacent the mixing chamber, and the sample not held by the magnet is directed to the waste chamber so that all magnetic beads, whether bound into a dual bead complex or unbound, remain in the mixing chamber. The magnetic beads are then directed to the capture area. One of a number of different valving arrangements can be used to control the flow. In still another aspect of the present invention, a bio-disc is produced for use with biological samples and is used in conjunction with a disc drive, such as a magneto-optical disc drive, that can form magnetic regions on a disc. In a magneto-optical disc and drive, magnetic regions can be formed in a highly controllable and precise manner. These regions may be employed advantageously to magnetically bind magnetic beads, including unbound magnetic capture beads or including dual bead complexes with magnetic capture beads. The magneto-optical disc drive can write to selected locations on the disc, and then use an optical reader to detect features located at those regions. The regions can be erased, thereby allowing the beads to be released.

In still another aspect, the invention includes a method for use with a bio-disc and drive including forming magnetic regions on the bio-disc, and providing magnetic beads to the discs so that the beads bind at the magnetic locations. The method preferably further includes detecting at the locations where the magnetic beads bind biological samples, preferably using reporter beads that are detectable, such as by fluorescence or optical event detection. The method can be formed in multiple stages in terms of time or in terms of location through the use of multiple chambers. The regions are written to and a sample is moved over the magnetic regions in order to capture magnetic beads. The regions can then be erased and released if desired. This method allows many different tests to be performed at one time, and can allow a level of interactivity between the user and the disc drives such that additional tests can be created during the testing process.

In yet another aspect, the invention provides for a method of evaluating a solid phase for use in a dual bead assay. The method includes the steps of selecting a test solid phase, binding a probe to the test solid phase in the presence or absence of a cross linking agent, determining the total amount of probe bound to the test solid phase in the presence or absence of a cross-linking agent, determining the amount of probe bound to the solid phase covalently, and calculating the percentage of probe bound covalently to the solid phase. The covalent conjugation efficiency required in a dual bead assay varies depending on the target concentration. In one particular embodiment of the present invention, at least 80% covalent binding efficiency is necessary for the solid phase to be suitable for use in a dual bead assay. The process of determining the probe conjugation efficiency is discussed below in Examples 3 and 4.

In certain embodiments thereof, the solid phase is a bead, particularly a magnetic bead. In other embodiments thereof, the solid phase is a surface on a bio disc. Probes that may be tested for binding to a particular solid phase include, but are not limited to, nucleic acids and proteins.

Also it is an aspect of the invention to provide for a method of conjugation for attaching capture DNA and reporter DNA to solid phase. The method of conjugation is an important factor in obtaining good conjugation efficiency. The conjugation efficiency of DNA attachment to any solid phase depends primarily on the quality of the solid phase and the method of conjugation. Various methods of conjugation were investigated employing different parameters such as number of conjugation steps. The pH of the buffer and the mixing mode were also evaluated. In a typical conjugation, the solid phase is first activated in the presence of the cross-linker EDC at acidic pH (0.1M MES buffer, pH 6.0). The DNA probe is then added and the conjugation is carried out for several hours at room temperature. The mode of mixing during conjugation could affect the conjugation efficiency significantly. Intermittent mixing of the tubes during conjugation gives a higher yield than continuous mixing. After conjugation, the unreacted carboxyl groups on the solid phase are blocked. Different blocking reagents were investigated. The blocking by 0.1M Tris-HCl at pH 7.5 is preferred as among those considered to be most efficient. The conjugated beads can be stored at 4° C. for as long as 2 months without any detectable activity loss.

It is another aspect of the invention to attach a double stranded probe to the beads and to select appropriate bead type. The use of double stranded probes in the conjugation increases the covalent attachment of probes to beads significantly. By using appropriate bead type and conjugation conditions, the covalent conjugation efficiency may be as high as 100%.

In this method, the covalent and non-covalent attachment of probes to beads is carried out in the presence or absence of chemical cross-linkers (such as EDC or EDAC). If the non-covalent attachment of probes to a particular bead is less than 10%, that bead is suitable for covalent conjugation of probes. After conjugation, if 100% covalent probe conjugation is desirable, then heat treatment of the beads will dispose of any remaining non-covalently bound probes.

A high covalent conjugation efficiency of DNA probes is essential in the sensitivity of the dual bead assay. Biotinylated single-stranded DNA probes may be used to determine the covalent conjugation efficiency of the probe binding. After the conjugation procedure, the amount of probes is quantified. This quantification represents the total amount of probes (covalent and non-covalent) bound to the beads. Then the beads are subjected to heat treatment to remove the non-covalently bound probes. The amount of remaining probes is then quantified. The percentage of non-covalent probes can be easily calculated from the data from quantification of the total probes and the covalent probes. Example 3 describes the procedure for quantification of the covalent conjugation efficiency of oligonucleotide probes.

In one principal embodiment of the present invention, the dual bead assay may include magnetic capture beads and fluorescent reporter beads. These beads are coated with capture probes and reporter probes respectively. The capture probes and reporter probes are complementary to the target sequence but not to each other. The capture beads are mixed with varying quantities of target DNA and allowed sufficient time to hybridize. Unbound target is removed from the solution by magnetic concentration of the magnetic beads. Fluorescent reporter beads are then allowed to bind to the captured target DNA. Unbound reporter beads are removed by magnetic concentration of the magnetic beads. Thus only in the presence of the target sequence, the magnetic capture beads bind to fluorescent reporter beads resulting in a dual bead assay.

The capture and reporter probes are covalently conjugated onto carboxylated capture beads and reporter beads via EDC conjugation. The use of magnetic beads in the capture of target DNA speeds up the washing steps and significantly facilitates the separation steps between bound and unbound. Furthermore, when the target concentration is limiting, each target molecule will hybridize to one reporter bead. One target molecule is not detectable by any existing technologies but a 1 μm or larger reporter bead can be easily detected and quantified by various methods. Therefore, the dual bead assay increases the sensitivity of the target capture tremendously.

Aspects of the present invention may be advantageously implemented on an analysis disc, modified optical disc, or bio-disc. The bio-disc may include a flow channel having target or capture zones, a return channel in fluid communication therewith, and in some embodiments a mixing chamber in fluid communication with the flow channel. The bio-disc may be implemented on an optical disc including an information encoding format such as CD, CD-R, or DVD or a modified version thereof. The bio-disc may include encoded information for performing, controlling, and post-processing the test or assay. For example, such encoded information may be directed to controlling the rotation rate of the disc. Depending on the test, assay, or investigational protocol, the rotation rate may be variable with intervening or consecutive sessions of acceleration, constant speed, and deceleration. These sessions may be closely controlled both as to speed, direction, and time of rotation to provide, for example, mixing, agitation, or separation of fluids and suspensions with agents, reagents or antibodies. Methods of manufacturing the optical bio-disc according to the present invention are also aspects relating thereto.

Development of a DNA based assay for a bio-disc including, for example, CD, CD-R, or DVD formats and variations thereof, includes attachment of micro-particles or beads to the disc surface as a detection method. These particles or beads are selected in size so that the read or interrogation beam of a disc drive or reader can “see” or detect a change of surface reflectivity caused by the particles.

A bio-disc drive assembly may be employed to rotate the disc, read and process any encoded information stored on the disc, and analyze the DNA samples in the flow channel of the bio-disc. The bio-disc drive is thus provided with a motor for rotating the bio-disc, a controller for controlling the rate of rotation of the disc, a processor for processing return signals form the disc, and an analyzer for analyzing the processed signals. The rotation rate of the motor is controlled to achieve the desired rotation of the disc. The bio-disc drive assembly may also be utilized to write information to the bio-disc either before, during, or after the test material in the flow channel and target zones is interrogated by the read beam of the drive and analyzed by the analyzer. The bio-disc may include encoded information for controlling the rotation rate of the disc, providing processing information specific to the type of DNA test to be conducted, and for displaying the results on a monitor associated with the bio-drive.

According to yet another aspect hereof, the invention is directed at the use of linkers in capture and reporter probes to increase target mediated binding and to reduce non-specific binding of capture beads to reporter beads. The use of magnetic beads in the capture of target DNA speeds up the washing steps and facilitates the separation steps between bound and unbound significantly. Furthermore, when the target concentration is limiting, each target molecule will hybridize to one reporter bead. One target molecule is not detectable by any existing technologies but a 1 μm or larger reporter bead can be easily detected and quantified by various methods. Therefore, the dual bead assay increases the sensitivity of the target capture tremendously. After target capture, specific binding of reporter beads can be detected by different methods. These methods include microscopic analysis, measurement of the fluorescent signal using a fluorimeter, or bead detection in an optical disc or CD-type reader.

It is a preferred embodiment to introduce linkers into the probes. The surface of the capture and reporter beads as shown by atomic force measurement has rough surfaces that would limit the accessibility of the probes to the target in solution. To increase the accessibility of the probes to the target DNA in solution, linkers were introduced to the capture and reporter probes. The increased accessibility of the probes with respect to the target DNA has a double effect. First, it reduces the non-specific binding of capture beads to reporter beads and second, it increases the target mediated binding several fold.

The apparatus and methods in embodiments of the present invention can be designed for use by an end-user, inexpensively, without specialized expertise and expensive equipment. The system can be made portable, and thus usable in remote locations where traditional diagnostic equipment may not generally be available. Other related aspects applicable to components of this assay system and signal acquisition methods are disclosed in commonly assigned and co-pending U.S. patent application Ser. No. 10/038,297 entitled “Dual Bead Assays Including Covalent Linkages For Improved Specificity And Related Optical Analysis Discs” filed Jan. 4, 2002; U.S. Provisional Application Serial No. 60/272,525 entitled “Biological Assays Using Dual Bead Multiplexing Including Optical Bio-Disc and Related Methods” filed Mar. 1, 2001; and U.S. Provisional Application Serial Nos. 60/275,643, 60/314,906, and 60/352,270 each entitled “Surface Assembly for Immobilizing Capture Agents and Dual Bead Assays Including Optical Bio-Disc and Methods Relating Thereto” respectively filed Mar. 14, 2001, Aug. 24, 2001, and Jan. 30, 2002. All of these applications are herein incorporated by reference in their entirety.

Other features and advantages of the present invention will become apparent from the following detailed description and accompanying drawing figures.

### BRIEF DESCRIPTION OF THE DRAWING FIGURES

Further objects of the present invention together with additional features contributing thereto and advantages accruing therefrom will be apparent from the following description of preferred embodiments of the present invention which are shown in the accompanying drawing figures with like reference numerals indicating like components throughout, wherein:

FIG. 1 is a perspective view of an optical disc system according to the present invention;

FIG. 2 is a block and pictorial diagram of an optical reading system according to embodiments of the present invention;

FIGS. 3A, 3B, and 3C are respective exploded, top, and perspective views of a reflective disc according to embodiments of the present invention;

FIGS. 4A, 4B, and 4C are respective exploded, top, and perspective views of a transmissive disc according to embodiments of the present invention;

FIG. 5A is a partial longitudinal cross sectional view of the reflective optical bio-disc shown in FIGS. 3A, 3B, and 3C illustrating a wobble groove formed therein;

FIG. 5B is a partial longitudinal cross sectional view of the transmissive optical bio-disc illustrated in FIGS. 4A, 4B, and 4C showing a wobble groove formed therein and a top detector;

FIG. 6A is a partial radial cross-sectional view of the disc illustrated in FIG. 5A;

FIG. 6B is a partial radial cross-sectional view of the disc illustrated in FIG. 5B;

FIGS. 7A, 8A, 9A, and 10A are schematic representations of a capture bead, a reporter bead, and a dual bead complex as utilized in conjunction with genetic assays;

FIGS. 7B, 8B, 9B, and 10B are schematic representations of a capture bead, a reporter bead, and a dual bead complex as employed in conjunction with immunochemical assays;

FIG. 11A is a pictorial representation of one embodiment of a method for producing genetic dual bead complex solutions;

FIG. 11B is a pictorial representation of one embodiment of a method for producing immunochemical dual bead complex solutions;

FIG. 12A is a pictorial representation of another embodiment of a method for producing genetic dual bead complex solutions;

FIG. 12B is a pictorial representation of another embodiment of a method for producing immunochemical dual bead complex solutions;

FIG. 13 is a longitudinal cross sectional view illustrating the disk layers in combination with a mixing or loading chamber;

FIG. 14 is a view similar to FIG. 13 showing the mixing chamber loaded with dual bead complex solution;

FIGS. 15A and 15B are radial cross sectional views of the disc and target zone illustrating one embodiment for binding of reporter beads to capture agents in a genetic assay;

FIGS. 16A and 16B are radial cross sectional views of the disc and target zone showing another embodiment for binding of reporter beads to capture agents in a genetic assay;

FIG. 17 is radial cross sectional view of the disc and target zone illustrating one embodiment for binding of capture beads to capture agents in a genetic assay;

FIG. 18 is radial cross sectional view of the disc and target zone depicting another embodiment for binding of capture beads to capture agents in a genetic assay;

FIGS. 19A, 19B, and 19C are partial cross sectional views illustrating one embodiment of a method according to this invention for binding the reporter bead of a dual bead complex to a capture layer in a genetic assay;

FIGS. 20A, 20B, and 20C are partial cross sectional views showing one embodiment of a method according to the present invention for binding the reporter bead of a dual bead complex to a capture layer in a immunochemical assay;

FIGS. 21A, 21B, and 21C are partial cross sectional views illustrating another embodiment of a method according to this invention for binding the reporter bead of a dual bead complex to a capture layer in a genetic assay;

FIGS. 22A, 22B, and 22C are partial cross sectional views presenting another embodiment of a method according to the invention for binding the reporter bead of a dual bead complex to a capture layer in a immunochemical assay;

FIGS. 23A and 23B are partial cross sectional views depicting one embodiment of a method according to the present invention for binding the capture bead of a dual bead complex to a capture layer in a genetic assay;

FIGS. 24A and 24B are partial cross sectional views showing another embodiment of a method according to this invention for binding the capture bead of a dual bead complex to a capture layer in a genetic assay;

FIGS. 25A-25D illustrate a method according to the present invention for detecting the presence of target DNA or RNA in a genetic sample utilizing an optical bio-disc;

FIGS. 26A-26D illustrate another method according to this invention for detecting the presence of target DNA or RNA in a genetic sample utilizing an optical bio-disc;

FIGS. 27A-27D illustrate a method according to the present invention for detecting the presence of a target antigen in a biological test sample utilizing an optical bio-disc;

FIG. 28A is a graphical representation of an individual 2.1 micron reporter bead and a 3 micron capture bead positioned relative to the tracks of an optical bio-disc according to the present invention;

FIG. 28B is a series of signature traces derived from the beads of FIG. 28A utilizing a detected signal from the optical drive according to the present invention;

FIG. 29A is a graphical representation of a 2.1 micron reporter bead and a 3 micron capture bead linked together in a dual bead complex positioned relative to the tracks of an optical bio-disc according to the present invention;

FIG. 29B is a series of signature traces derived from the dual bead complex of FIG. 29A utilizing a detected signal from the optical drive according to this invention;

FIG. 30A is a bar graph showing results from a dual bead assay according to the present invention;

FIG. 30B is a graph showing a standard curve demonstrating the detection limit for fluorescent beads detected with a flourimeter;

FIG. 30C is a pictorial representation demonstrating the formation of the dual bead complex;

FIG. 31 is a bar graph showing the sensitivity of the disc drive detection of the dual bead complex;

FIG. 32 is a schematic representation of combining beads for dual bead assay multiplexing according to embodiments of the present invention;

FIG. 33A is a schematic representation of a fluidic circuit according to the present invention utilized in conjunction with a magnetic field generator to control movement of magnetic beads;

FIGS. 33B-33D are schematics of a first fluidic circuit that implements the valving structure of FIG. 33A according to one embodiment of fluid transport aspects of the present invention;

FIGS. 34A-34C are schematics of a second fluidic circuit that implements the valving structure of FIG. 33A according to another embodiment of fluid transport aspects of the present invention;

FIG. 35 is a perspective view of a the magnetic field generator and a disc including one embodiment of a fluidic circuit employed in conjunction with magnetic beads according to this invention;

FIGS. 36A, 36B, and 36C are plan views illustrating a method of separation and detection for dual bead assays using the fluidic circuit shown in FIG. 35;

FIG. 37 is a perspective view of a magneto-optical bio-disc showing magnetic regions, magnetically bound capture beads, and the formation of dual bead complexes according to another aspect of the present invention.

FIG. 38 is a schematic presenting a method for evaluating a solid phase for covalent conjugation of a probe;

FIG. 39 is a schematic detailing various steps in the quantification of covalently-bound and non-covalently bound probes to a solid substrate;

FIG. 40A is a graphic presentation of experimental results of various tests of magnetic bead carriers for covalent linkage of a probe;

FIG. 40B is a graphic presentation of experimental results of various tests of fluorescent bead carriers for covalent linkage of a probe;

FIG. 41A is a pictorial representation illustrating the structural differences between single-stranded and double-stranded DNA that are relevant to their use as probes;

FIG. 41B is a graphic presentation of results of an experiment designed to evaluate the binding properties of single-stranded and double-stranded DNA to a solid phase;

FIG. 42A is graphic presentation of enzyme assay results of a screen of two different capture beads for use in a dual bead assay, these results indicating that both of the tested beads bind a similar amount of target regardless of whether the probe is bound covalently or non-covalently;

FIG. 42B is a graphic presentation of results of a dual bead assay designed to examine the number of reporter beads captured by two different capture beads, these results indicate that covalent bonding of the probe to the capture bead greatly improves assay sensitivity;

FIG. 43 is a graphic presentation demonstrating that the introduction of PEG linkers into probes significantly improves target mediated binding;

FIG. 44 is a bar graph presentation illustrating probe density determination employing 3 μm beads;

FIG. 45 is a bar graph presentation demonstrating the pretreatment of the beads with various detergents including salmon sperm DNA which reduced nonspecific binding by over 10 fold.

### DETAILED DESCRIPTION OF THE PREFERRED EMBODIMENTS

The following description of the present invention relates to optical analysis discs, disc drive systems, and assay chemistries and techniques. The invention further relates to alternate magneto-optical drive systems, MO bio-discs, and related processing methods.

Disc Drive System and Related Optical Analysis Discs

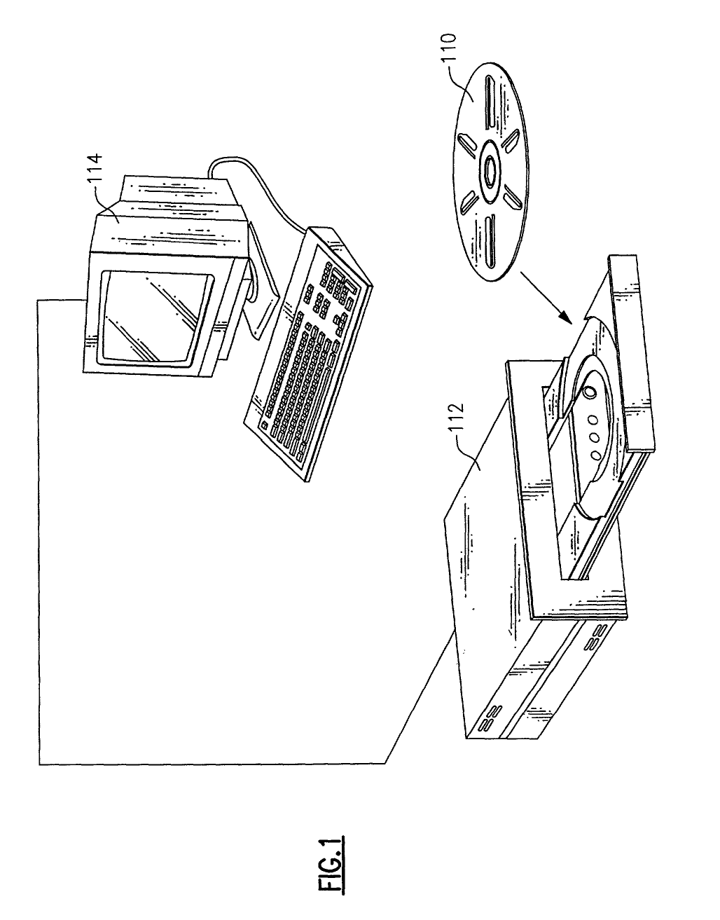

With reference now to FIG. 1, there is shown a perspective view of an optical bio-disc 110 for use in an optical disc drive 112. Drive 112, in conjunction with software in the drive or associated with a separate computer, can cause images, graphs, or output data to be displayed on display monitor 114. As indicated below, there are different types of discs and drives that can be used. The disc drive can be in a unit separate from a controlling computer, or provided in a bay within a computer. The device can be made as portable as a laptop computer, and thus usable with battery power and in remote locations not generally served by advanced diagnostic equipment. The drive is preferably a conventional drive with minimal or no hardware modification, but can be a dedicated bio-disc drive. Further details regarding these types of drive systems and related signal processing methods are disclosed in, for example, commonly assigned and co-pending U.S. patent application Ser. No. 09/378,878 entitled “Methods and Apparatus for Analyzing Operational and Non-operational Data Acquired from Optical Discs” filed Aug. 23, 1999; U.S. Provisional Patent Application Serial No. 60/150,288 entitled “Methods and Apparatus for Optical Disc Data Acquisition Using Physical Synchronization Markers” filed Aug. 23, 1999; U.S. patent application Ser. No. 09/421,870 entitled “Trackable Optical Discs with Concurrently Readable Analyte Material” filed Oct. 26, 1999; U.S. patent application Ser. No. 09/643,106 entitled “Methods and Apparatus for Optical Disc Data Acquisition Using Physical Synchronization Markers” filed Aug. 21, 2000; U.S; and U.S. patent application Ser. No. 10/043,688 entitled “Optical Disc Analysis System Including Related Methods For Biological and Medical Imaging” filed Jan. 10, 2002. These applications are herein incorporated by reference in their entirety.

Optical bio-disc 110 for use with embodiments of the present invention may have any suitable shape, diameter, or thickness, but preferably is implemented on a round disc with a diameter and a thickness similar to those of a compact disc (CD), a recordable CD (CD-R), CD-RW, a digital versatile disc (DVD), DVD-R, DVD-RW, or other standard optical disc format. The disc may include encoded information, preferably in a known format, for performing, controlling, and post-processing a test or assay, such as information for controlling the rotation rate and direction of the disc, timing for rotation, stopping and starting, delay periods, locations of samples, position of the light source, and power of the light source. Such encoded information is referred to generally here as operational information.

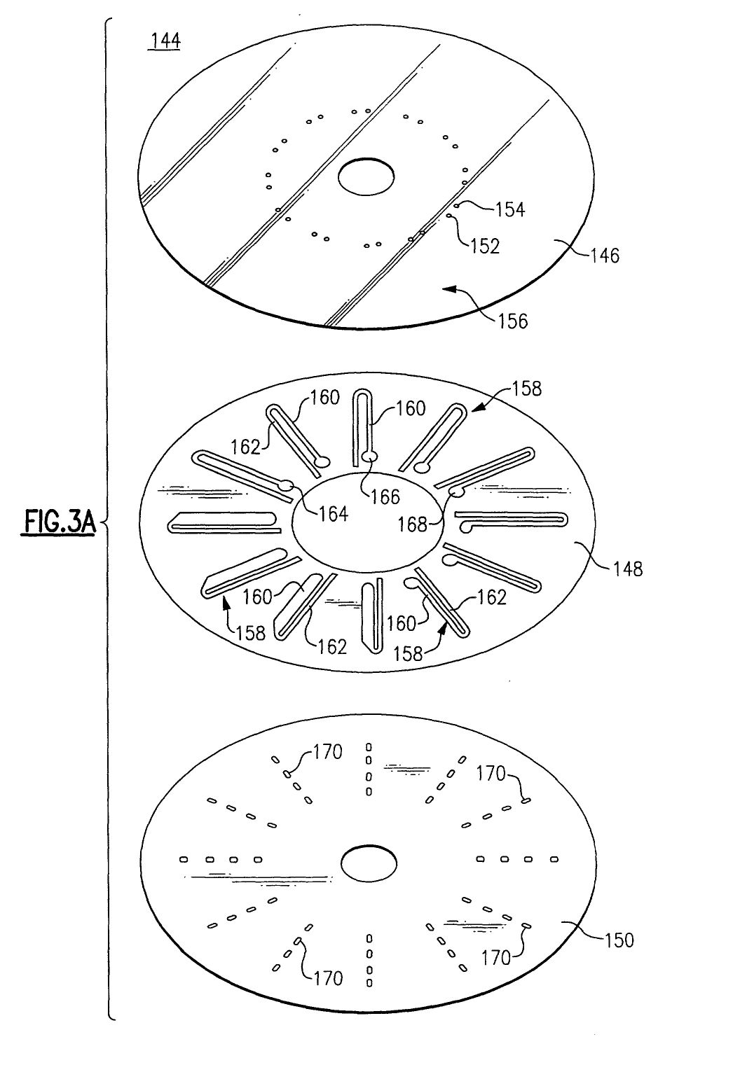

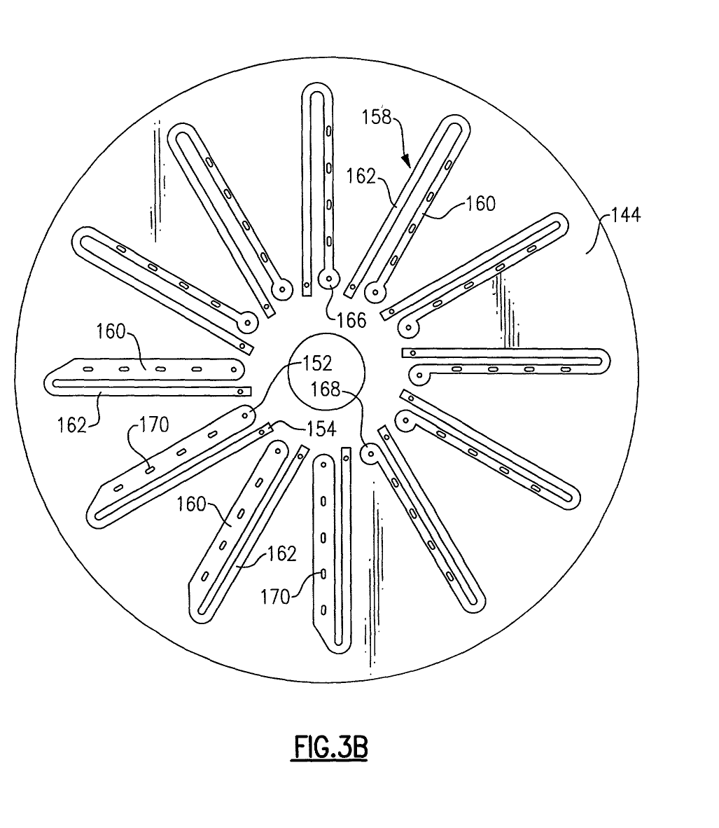

The disc may be a reflective disc, as shown in FIGS. 3A-3C, a transmissive disc, FIGS. 4A-4C, or some combination of reflective and transmissive. In a reflective disc, an incident light beam is focused onto the disc (typically at a reflective surface where information is encoded), reflected, and returned through optical elements to a detector on the same side of the disc as the light source. In a transmissive disc, light passes through the disc (or portions thereof) to a detector on the other side of the disc from the light source. In a transmissive portion of a disc, some light may also be reflected and detected as reflected light.

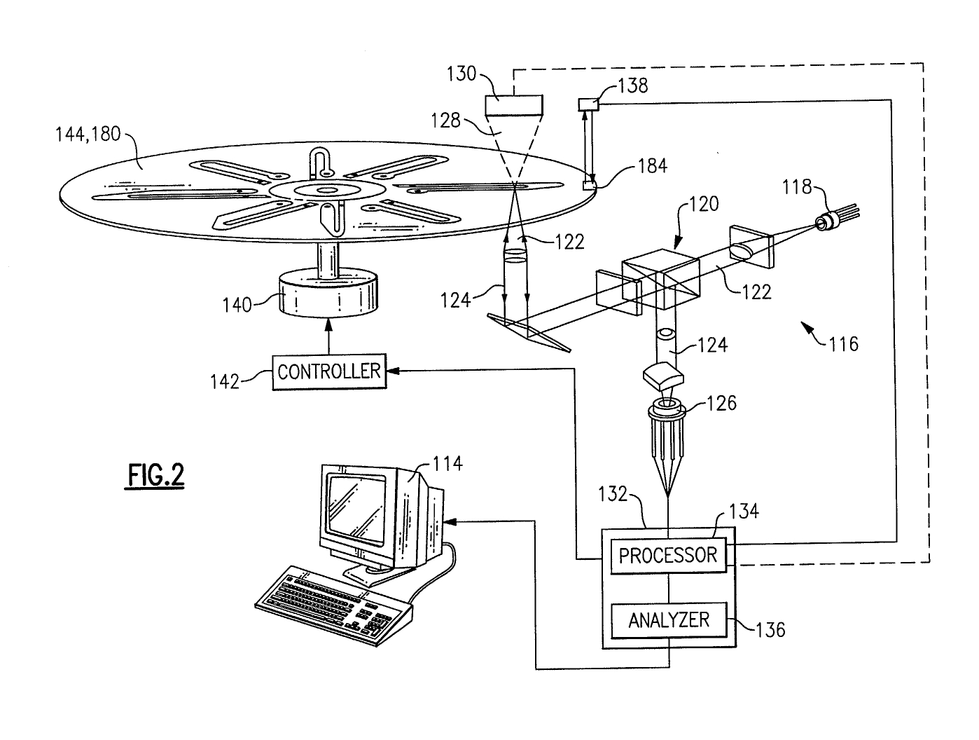

FIG. 2 shows an optical disc reader system 116. This system may be a conventional reader for CD, CD-R, DVD, or other known comparable format, a modified version of such a drive, or a completely distinct dedicated device. The basic components are a motor for rotating the disc, a light system for providing light, and a detection system for detecting light.

With reference now generally to FIGS. 2 through 4C, a light source 118 provides light to optical components 120 to produce an incident light beam 122. In the case of reflective disc 144, FIGS. 3A-3C, a return beam 124 is reflected from either reflective surface 156, 174, or 186, FIGS. 3C and 4C. Return beam 124 is provided back to optical components 120, and then to a bottom detector 126. In this type of disc, the return beam may carry operational information or other encoded data as well as characteristic information about the investigational feature or test sample under study.

For transmissive disc 180, FIGS. 4A-4C, some of the energy from the incident beam 122 will undergo a light/matter interaction with an investigational feature or test sample and then proceed through the disc as a transmitted beam 128 that is detected by a top detector 130. For a transmissive disc including a semi-reflective layer 186 (FIG. 4C) as the operational layer, some of the energy from the incident beam 122 will also reflect from the operational layer as return beam 124 which carries operational information or stored data. Optical components 120 can include a lens, a beam splitter, and a quarter wave plate that changes the polarization of the light beam so that the beam splitter directs a reflected beam through the lens to focus the reflected beam onto the detector. An astigmatic element, such as a cylindrical lens, may be provided between the beam splitter and detector to introduce astigmatism in the reflected light beam. The light source can be controllable to provide variable wavelengths and power levels over a desired range in response to data introduced by the user or read from the disc. This controllability is especially useful when it is desired to detect multiple different structures that fluoresce at different wavelengths.

Now with continuing reference to FIG. 2, it is shown that data from detector 126 and/or detector 130 is provided to a computer 132 including a processor 134 and an analyzer 136. An image or output results can then be provided to a monitor 114. Computer 132 can represent a desktop computer, programmable logic, or some other processing device, and also can include a connection (such as over the Internet) to other processing and/or storage devices. A drive motor 140 and a controller 142 are provided for controlling the rotation rate and direction or rotation of disc the 144 or 180. Controller 142 and the computer 132 with processor 134 can be in remote communication or implemented in the same computer. Methods and systems for reading such a disc are also shown in Gordon, U.S. Pat. No. 5,892,577, which is incorporated herein by reference.

The detector can be designed to detect all light that reaches the detector, or though its design or an external filter, light only at specific wavelengths. By making the detector controllable in terms of the detectable wavelength, beads or other structures that fluoresce at different wavelengths can be separately detected.

A hardware trigger sensor 138 may be used with either a reflective disc 144 or transmissive disc 180. Triggering sensor 138 provides a signal to computer 132 (or to some other electronics) to allow for the collection of data by processor 134 only when incident beam 122 is on a target zone or inspection area. Alternatively, software read from a disc can be used to control data collection by processor 134 independent of any physical marks on the disc. Such software or logical triggering is discussed in further detail in commonly assigned and co-pending U.S. Provisional Application Serial No. 60/352,625 entitled “Logical Triggering Methods And Apparatus For Use With Optical Analysis Discs And Related Disc Drive Systems” filed Jan. 28, 2002, which is herein incorporated by reference in its entirety.

The substrate layer of the optical analysis disc may be impressed with a spiral track that starts at an innermost readable portion of the disc and then spirals out to an outermost readable portion of the disc. In a non-recordable CD, this track is made up of a series of embossed pits with varying length, each typically having a depth of approximately one-quarter the wavelength of the light that is used to read the disc. The varying lengths and spacing between the pits encode the operational data. The spiral groove of a recordable CD-like disc has a detectable dye rather than pits. This is where the operation information, such as the rotation rate, is recorded. Depending on the test, assay, or investigational protocol, the rotation rate may be variable with intervening or consecutive periods of acceleration, constant speed, and deceleration. These periods may be closely controlled both as to speed and time of rotation to provide, for example, mixing, agitation, or separation of fluids and suspensions with agents, reagents, antibodies, or other materials. Different optical analysis disc and bio-disc designs that may be utilized with the present invention, or readily adapted thereto, are disclosed, for example, in commonly assigned, copending U.S. patent application Ser. No. 09/999,274 entitled “Optical Bio-discs with Reflective Layers” filed on Nov. 15, 2001; U.S. patent application Serial No. 10/005,313 entitled “Optical Discs for Measuring Analytes” filed Dec. 7, 2001; U.S. patent application Ser. No. 10/006,371 entitled “Methods for Detecting Analytes Using Optical Discs and Optical Disc Readers” filed Dec. 10, 2001; U.S. patent application Ser. No. 10/006,620 entitled “Multiple Data Layer Optical Discs for Detecting Analytes” filed Dec. 10, 2001; and U.S. patent application Ser. No. 10/006,619 entitled “Optical Disc Assemblies for Performing Assays” filed Dec. 10, 2001, which are all herein incorporated by reference in their entirety.

Numerous designs and configurations of an optical pickup and associated electronics may be used in the context of the embodiments of the present invention. Further details and alternative designs for compact discs and readers are described in Compact Disc Technology, by Nakajima and Ogawa, IOS Press, Inc. (1992); The Compact Disc Handbook, Digital Audio and Compact Disc Technology, by Baert et al. (eds.), Books Britain (1995); and CD-Rom Professional's CD-Recordable Handbook: The Complete Guide to Practical Desktop CD, Starrett et al. (eds.), ISBN:0910965188 (1996); all of which are incorporated herein in their entirety by reference.

The disc drive assembly is thus employed to rotate the disc, read and process any encoded operational information stored on the disc, and analyze the liquid, chemical, biological, or biochemical investigational features in an assay region of the disc. The disc drive assembly may be further utilized to write information to the disc either before, during, or after the material in the assay zone is analyzed by the read beam of the drive. In alternate embodiments, the disc drive assembly is implemented to deliver assay information through various possible interfaces such as via Ethernet to a user, over the Internet, to remote databases, or anywhere such information could be advantageously utilized. Further details relating to this type of disc drive interfacing are disclosed in commonly assigned copending U.S. patent application Ser. No. 09/986,078 entitled “Interactive System For Analyzing Biological Samples And Processing Related Information And The Use Thereof” filed Nov. 7, 2001, which is incorporated herein by reference in its entirety.

Referring now specifically to FIGS. 3A, 3B, and 3C, the reflective disc 144 is shown with a cap 146, a channel layer 148, and a substrate 150. The channel layer 148 may be formed by a thin-film adhesive member. Cap 146 has inlet ports 152 for receiving samples and vent ports 154. Cap 146 may be formed primarily from polycarbonate, and may be coated with a cap reflective layer 156 on the bottom thereof. Reflective layer 156 is preferably made from a metal such as aluminum or gold.

Channel layer 148 defines fluidic circuits 158 by having desired shapes cut out from channel layer 148. Each fluidic circuit 158 preferably has a flow channel 160 and a return channel 162, and some have a mixing chamber 164. A mixing chamber 166 can be symmetrically formed relative to the flow channel 160, while an off-set mixing chamber 168 is formed to one side of the flow channel 160. Fluidic circuits 158 are rather simple in construction, but a fluidic circuit can include other channels and chambers, such as preparatory regions or a waste region, as shown, for example, in U.S. Pat. No. 6,030,581, which is incorporated herein by reference, and can include valves and other fluid control structures. Channel layer 148 can include adhesives for bonding to the substrate and to the cap.

Substrate 150 has a plastic layer 172, and has target zones 170 formed as openings in a substrate reflective layer 174 deposited on the top of layer 172. In this embodiment, reflective layer 174, best illustrated in FIG. 3C, is used to encode operational information. Plastic layer 172 is preferably formed from polycarbonate. Target zones 170 may be formed by removing portions of the substrate reflective layer 174 in any desired shape, or by masking target zone areas before applying substrate reflective layer 174. The substrate reflective layer 174 is preferably formed from a metal, such as aluminum or gold, and can be configured with the rest of the substrate to encode operational information that is read with incident light, such as through a wobble groove or through an arrangement of pits. Light incident from under substrate 150 thus is reflected by layer 174, except at target zones 170, where it is reflected by layer 156. Target zones are where investigational features are detected. If the target zone is a location where an antibody, strand of DNA, or other material that can bind to a target is located, the target zone can be referred to as a capture zone.

With reference now particularly to FIG. 3C, optical disc 144 is cut away to illustrate a partial cross-sectional perspective view. An active layer 176 is formed over substrate reflective layer 174. Active layer 176 may generally be formed from nitrocellulose, polystyrene, polycarbonate, gold, activated glass, modified glass, or a modified polystyrene such as, for example, polystyrene-co-maleic anhydride. In this embodiment, channel layer 148 is situated over active layer 174.

In operation, samples can be introduced through inlet ports 152 of cap 146. When rotated, the sample moves outwardly from inlet port 152 along active layer 176. Through one of a number of biological or chemical reactions or processes, detectable features, referred to as investigational features, may be present in the target zones. Examples of such processes are shown in the incorporated U.S. Pat. No. 6,030,581 and commonly assigned, co-pending U.S. patent application Ser. No. 09/988,728 entitled “Methods And Apparatus For Detecting And Quantifying Lymphocytes With Optical Biodiscs” filed Nov. 16, 2001; and U.S. patent application Ser. No. 10/035,836 entitled “Surface Assembly For Immobilizing DNA Capture Probes And Bead-Based Assay Including Optical Bio-Discs And Methods Relating Thereto” filed Dec. 21, 2001, both of which are herein incorporated by reference in their entireties.

The investigational features captured within the target zones, by the capture layer with a capture agent, may be designed to be located in the focal plane coplanar with reflective layer 174, where an incident beam is typically focused in conventional readers. Alternatively, the investigational features may be captured in a plane spaced away from the focal plane. The former configuration is referred to as a “proximal” type disc, and the latter a “distal” type disc.

Referring to FIGS. 4A, 4B, and 4C, it is shown that one particular embodiment of the transmissive optical disc 180 includes a clear cap 182, a channel layer 148, and a substrate 150. The clear cap 182 includes inlet ports 152 and vent ports 154 and is preferably formed mainly from polycarbonate. Trigger marks 184 may be included on the cap 182. Channel layer 148 has fluidic circuits 158, which can have structure and use similar to those described in conjunction with FIGS. 3A, 3B, and 3C. Substrate 150 may include target zones 170, and preferably includes a polycarbonate layer 172. Substrate 150 may, but need not, have a thin semi-reflective layer 186 deposited on top of layer 172. Semi-reflective layer 186 is preferably significantly thinner than substrate reflective layer 174 on substrate 150 of reflective disc 144 (FIGS. 3A-3C). Semi-reflective layer 186 is preferably formed from a metal, such as aluminum or gold, but is sufficiently thin to allow a portion of an incident light beam to penetrate and pass through layer 186, while some of the incident light is reflected back. A gold film layer, for example, is 95% reflective at a thickness greater than about 700 Å, while the transmission of light through the gold film is about 50% transmissive at approximately 100 Å.

FIG. 4C is a cut-away perspective view of transmissive disc 180. The semi-reflective nature of layer 186 makes its entire surface potentially available for target zones, including virtual zones defined by trigger marks or encoded data patterns on the disc. Target zones 170 may also be formed by marking the designated area in the indicated shape or alternatively in any desired shape. Markings to indicate target zone 170 may be made on semi-reflective layer 186 or on a bottom portion of substrate 150 (under the disc). Target zones 170 may be created by silk screening ink onto semi-reflective layer 186.

An active layer 176 is applied over semi-reflective layer 186. Active layer 176 may be formed from the same materials as described above in conjunction with layer 176 (FIG. 3C) and serves substantially the same purpose when a sample is provided through an opening in disc 180 and the disc is rotated. In transmissive disc 180, there is no reflective layer, on the clear cap 182, comparable to reflective layer 156 in reflective disc 144 (FIG. 3C).

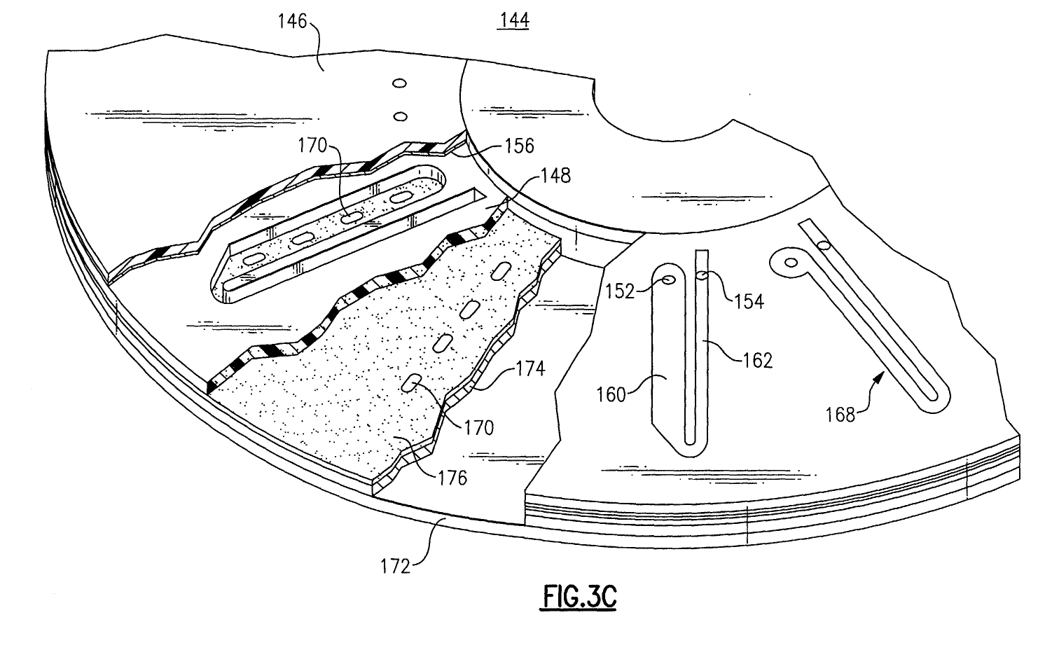

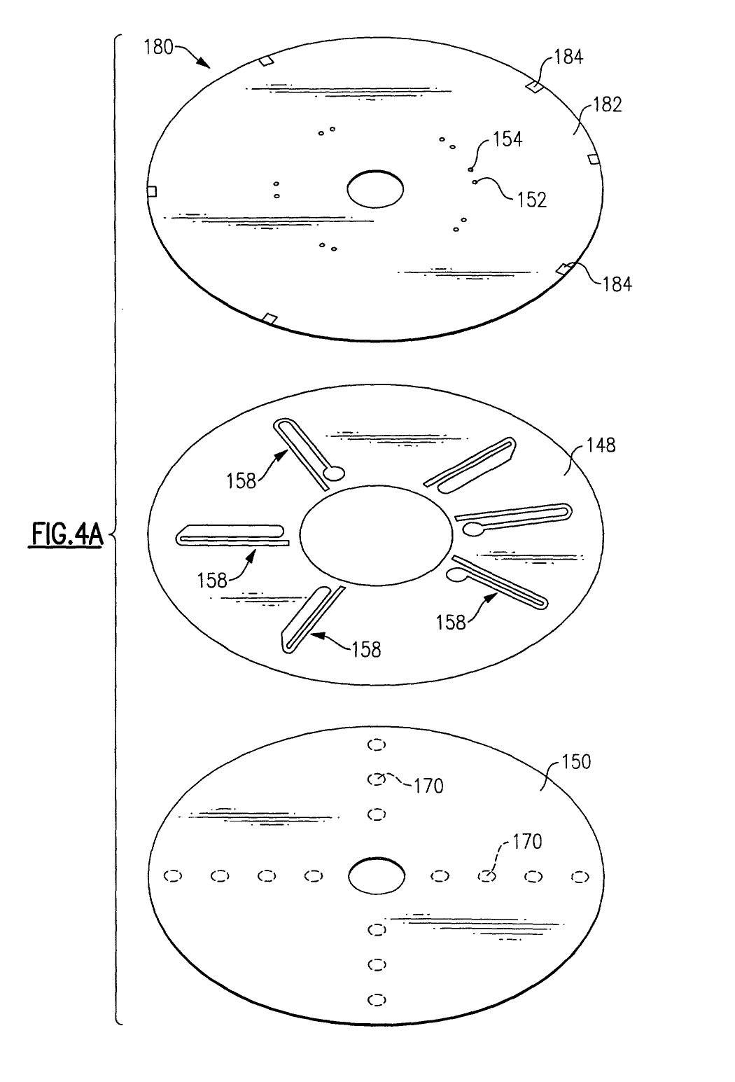

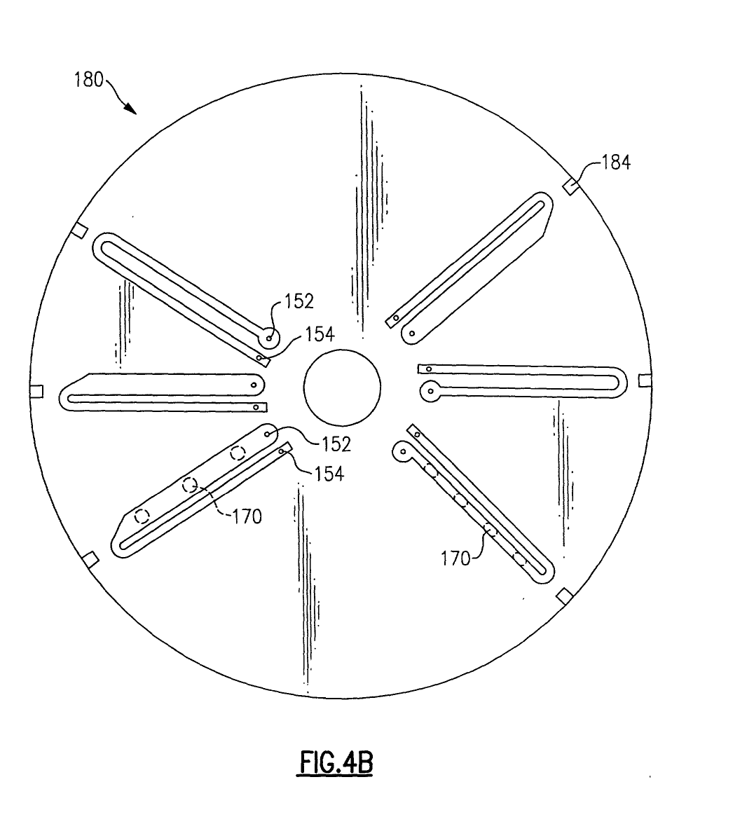

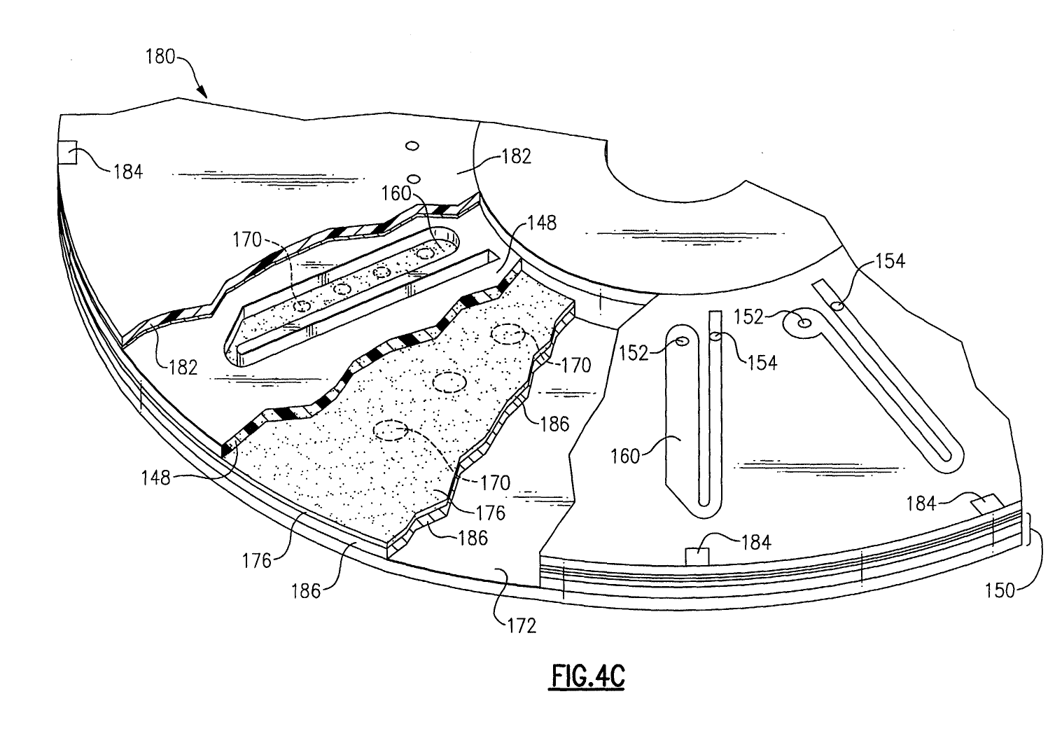

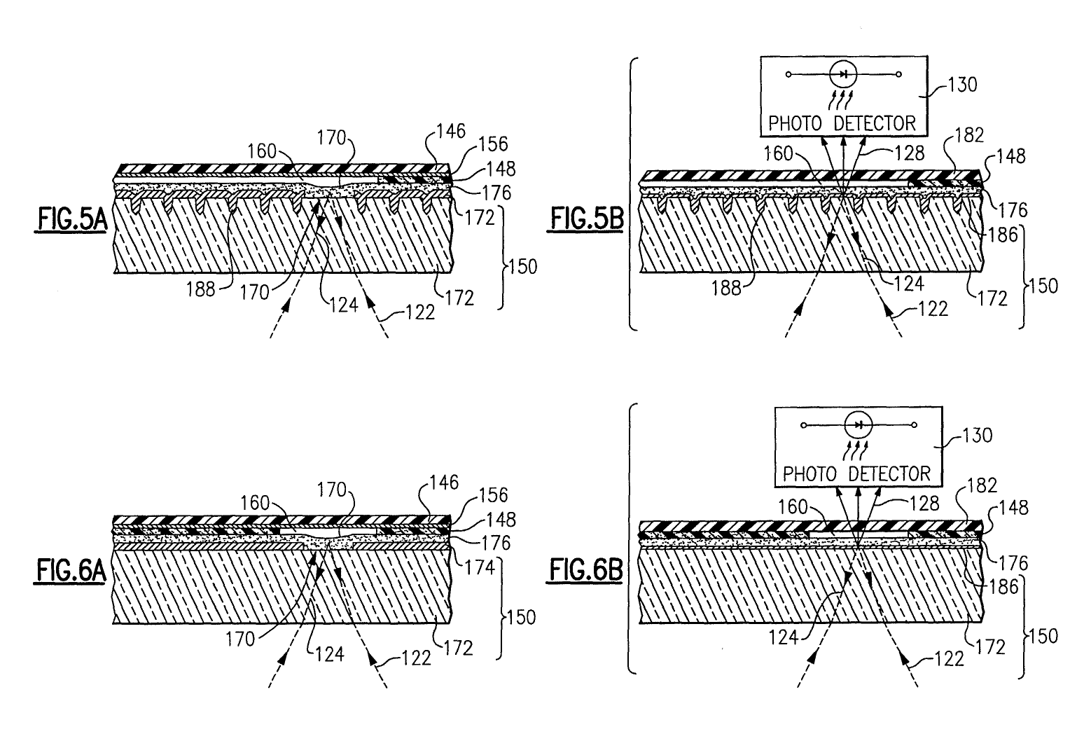

Referring now to FIG. 5A, there is shown a cross sectional view taken across the tracks of the reflective disc embodiment 144 (FIGS. 3A-3C) of the bio-disc 110 (FIG. 1) according to the present invention. As illustrated, this view is taken longitudinally along a radius and flow channel of the disc. FIG. 5A includes the substrate 150 which is composed of a plastic layer 172 and a substrate reflective layer 174. In this embodiment, the substrate 150 includes a series of grooves 188. The grooves 188 are in the form of a spiral extending from near the center of the disc toward the outer edge. The grooves 188 are implemented so that the interrogation or incident beam 122 may track along the spiral grooves 188 on the disc. This type of groove 188 is known as a “wobble groove”. The groove 188 is formed by a bottom portion having undulating or wavy side walls. A raised or elevated portion separates adjacent grooves 188 in the spiral. The reflective layer 174 applied over the grooves 188 in this embodiment is, as illustrated, conformal in nature. FIG. 5A also shows the active layer 176 applied over the reflective layer 174. As shown in FIG. 5A, the target zone 170 is formed by removing an area or portion of the reflective layer 174 at a desired location or, alternatively, by masking the desired area prior to applying the reflective layer 174. As further illustrated in FIG. 5A, the plastic adhesive member or channel layer 148 is applied over the active layer 176. FIG. 5A also shows the cap portion 146 and the reflective surface 156 associated therewith. Thus, when the cap portion 146 is applied to the plastic adhesive member 148 including the desired cut-out shapes, the flow channel 160 is thereby formed.

FIG. 5B is a cross sectional view, similar to that illustrated in FIG. 5A, taken across the tracks of the transmissive disc embodiment 180 (FIGS. 4A-4C) of the bio-disc 110 (FIG. 1) according to the present invention. This view is taken longitudinally along a radius and flow channel of the disc. FIG. 5B illustrates the substrate 150 that includes the thin semi-reflective layer 186. This thin semi-reflective layer 186 allows the incident or interrogation beam 122, from the light source 118 (FIG. 2), to penetrate and pass through the disc to be detected by the top detector 130, while some of the light is reflected back in the form of the return beam 124. The thickness of the thin semi-reflective layer 186 is determined by the minimum amount of reflected light required by the disc reader to maintain its tracking ability. The substrate 150 in this embodiment, like that discussed in FIG. 5A, includes the series of grooves 188. The grooves 188 in this embodiment are also preferably in the form of a spiral extending from near the center of the disc toward the outer edge. The grooves 188 are implemented so that the interrogation beam 122 may track along he spiral. FIG. 5B also shows the active layer 176 applied over the thin semi-reflective layer 186. As further illustrated in FIG. 5B, the plastic adhesive member or channel layer 148 is applied over the active layer 176. FIG. 5B also shows the clear cap 182. Thus, when the clear cap 182 is applied to the plastic adhesive member 148 including the desired cut-out shapes, the flow channel 160 is thereby formed and a part of the incident beam 122 is allowed to pass therethrough substantially unreflected. The amount of light that passes through can then be detected by the top detector 130.

FIG. 6A is a view similar to FIG. 5A but taken perpendicularly to a radius of the disc to illustrate the reflective disc and the initial refractive property thereof when observing the flow channel 160 from a radial perspective. In a parallel comparison manner, FIG. 6B is a similar view to FIG. 5B but taken perpendicularly to a radius of the disc to represent the transmissive disc and the initial refractive property thereof when observing the flow channel 160 from a radial perspective. Grooves 188 are not seen in FIGS. 5A and 5B since the sections are cut along the grooves 188. FIGS. 6A and 6B show the presence of the narrow flow channel 160 that is situated perpendicular to the grooves 188 in these embodiments. FIGS. 5A, 5B, 6A, and 6B show the entire thickness of the respective reflective and transmissive discs. In these views, the incident beam 122 is illustrated initially interacting with the substrate 150 which has refractive properties that change the path of the incident beam as shown to provide focusing of the beam 122 on the reflective layer 174 or the thin semi-reflective layer 186.

Assay Chemistries and Dual Bead Formation

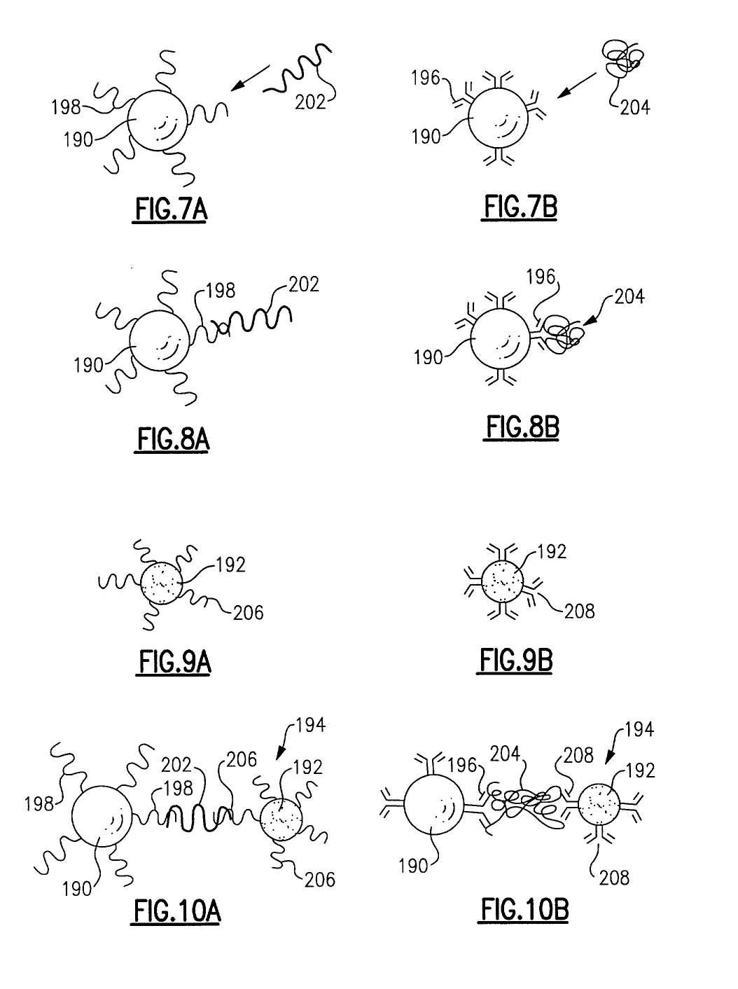

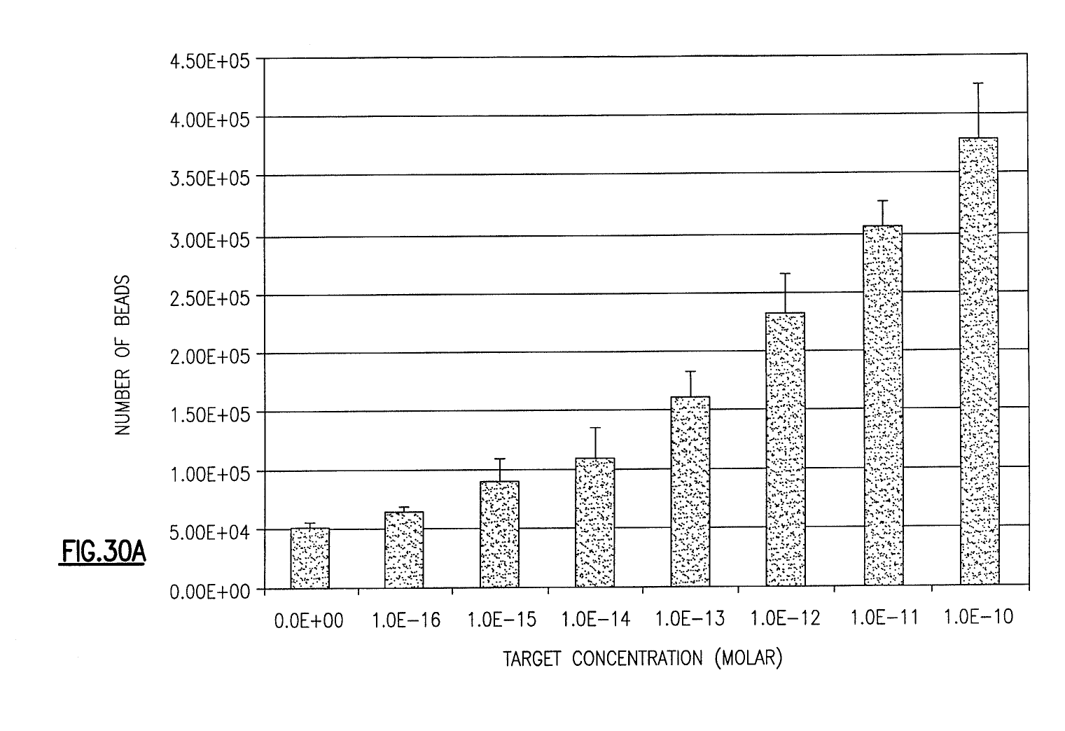

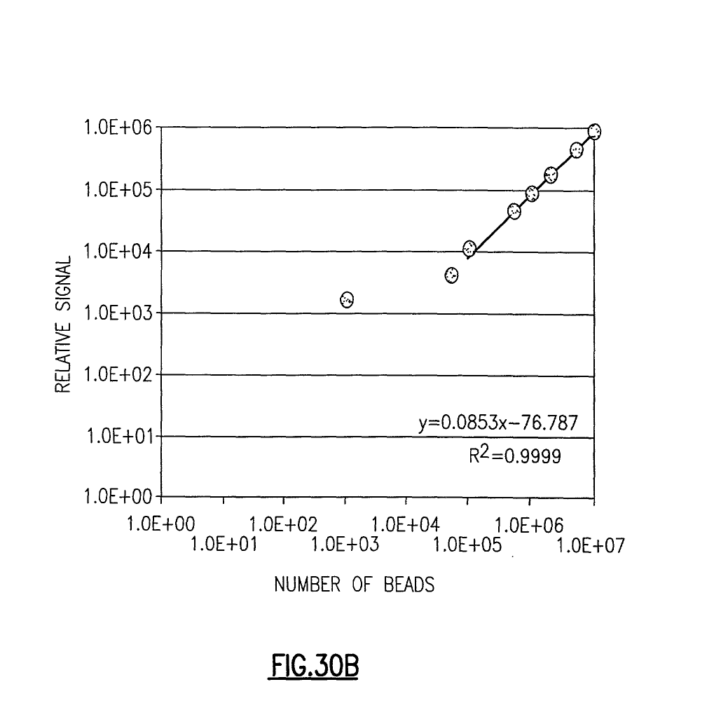

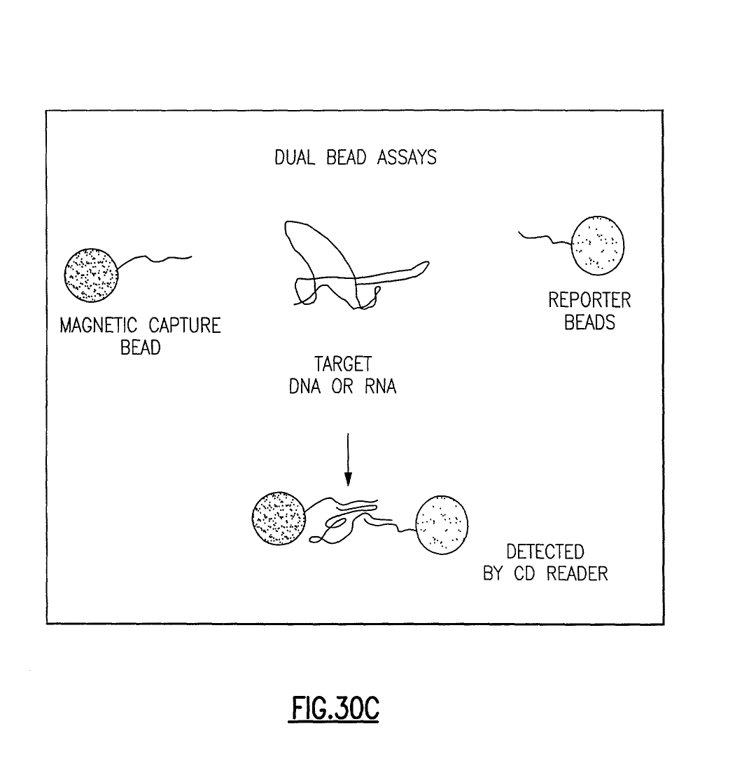

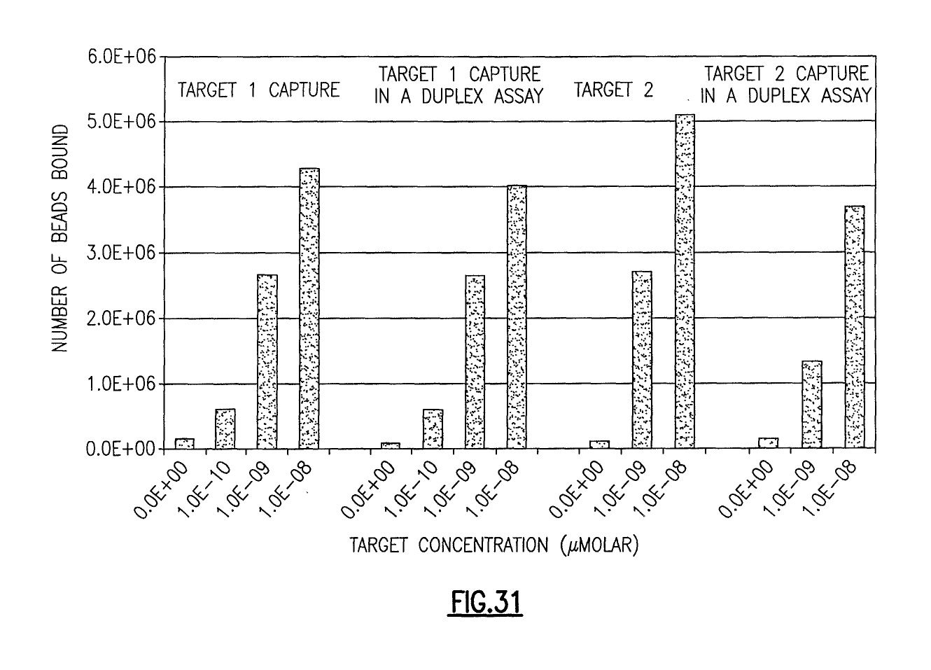

Referring now to FIGS. 7A-10A and 7B-10B, there is shown a capture bead 190, a reporter bead 192, and the formation of a dual bead complex 194. Capture bead 190 can be used in conjunction with a variety of different assays including biological assays such as immunoassays (FIGS. 7B-10B), molecular assays, and more specifically genetic assays (FIGS. 7A-10A). In the case of immunoassays, antibody transport probes 196 are conjugated onto the beads. Antibody transport probes 196 include proteins, such as antigens or antibodies, implemented to capture protein targets. In the case of molecular assays, oligonucleotide transport probes 198 would be conjugated onto the beads. Oligonucleotide transport probes 198 include nucleic acids such as DNA or RNA implemented to capture genetic targets. The dual bead formation as implemented in a genetic assay using single probes on each bead is also illustrated in FIG. 30C below.

As shown in FIG. 7A, a target agent such as target DNA or RNA 202, obtained from a test sample, is added to a capture bead 190 coated with oligonucleotide transport probes 198. In this implementation, transport probes 198 are formed from desired sequences of nucleic acids. Aspects relating to DNA probe conjugation onto solid phase of this system of assays are discussed in further detail in commonly assigned and co-pending U.S. Provisional Application Serial No. 60/278,685 entitled “Use of Double Stranded DNA for Attachment to Solid Phase to Reduce Non-Covalent Binding” filed Mar. 26, 2001. This application is herein incorporated by reference in its entirety.

As shown in FIG. 7B, a target agent such as target antigen 204 from a test sample is added to a capture bead 190 coated with antibody transport probes 196. In this alternate implementation, the transport probes 196 are formed from proteins such as antibodies.

Capture bead 190 has a characteristic that allows it to be isolated from a material suspension or solution. The capture bead may be selected based upon a desired size, and a preferred way to make it isolatable is for it to be magnetic.

FIG. 8A illustrates the binding of target DNA or RNA 202 to complementary transport probes 198 on capture bead 190 in the genetic assay implementation of the present invention. FIG. 8B shows an immunoassay version of FIG. 8A, transport probes 196 can alternatively include antibodies or antigens for binding to a target protein 204.

FIG. 9A shows a reporter bead 192 coated with oligonucleotide signal probes 206 complementary to target agent 202 (see FIG. 8A). Reporter bead 192 is selected based upon a desired size and the material properties for detection and reporting purposes. In one specific embodiment a 2.1 micron polystyrene bead is employed. Signal probes 206 can be strands of DNA or RNA to capture target DNA or RNA.

FIG. 9B illustrates a reporter bead 192 coated with antibody signal probes 208 that bind to the target agent 204 as shown in FIG. 8B. Reporter bead 192 is selected based upon a desired size and the material properties for detection and reporting purposes. This may also preferably include a 2.1 micron polystyrene bead. Signal probes 208 can be antigens or antibodies implemented to capture protein or glycoportein targets.

FIG. 10A is a pictorial representation of a dual bead complex 194 that can be formed from capture bead 190 with probe 198, target agent 202, and reporter bead 192 with probe 206. Probes 198 and 206 conjugated on capture bead 190 and reporter bead 192, respectively, have sequences complementary to the target agent 202, but not to each other. Further details regarding target agent detection and methods of reducing non-specific binding of target agents to beads are discussed in commonly assigned and co-pending U.S. Provisional Application Serial No. 60/278,106 entitled “Dual Bead Assays Including Use of Restriction Enzymes to Reduce Non-Specific Binding” filed Mar. 23, 2001; and U.S. Provisional Application Serial No. 60/278,110 entitled “Dual Bead Assays Including Use of Chemical Methods to Reduce Non-Specific Binding” filed Mar. 23, 2001, which are both incorporated herein by reference in their entirety.

FIG. 10B is a pictorial representation of the immunoassay version of a dual bead complex 194 that can be formed from capture bead 190 with probe 196, target agent 204, and reporter bead 192 with probe 208. Probes 196 and 208 conjugated on capture bead 190 and reporter bead 192, respectively, only bind to the target agent 202, and not to each other.

In an alternative embodiment of the current system of assays, target agent binding efficiency and specificity may be enhanced by using a cleavable spacer that temporarily links the reporter bead 192 and capture bead 190. The dual bead complex formed by the cleavable spacer essentially places the transport probe and the signal probe in close proximity to each other thus allowing more efficient target binding to both probes. Once the target agent is bound to the probes the spacer may then be cleaved permitting the bound target agent to retain the dual bead structure. The use of cleavable spacers in dual bead assay systems is disclosed in further detail in commonly assigned and co-pending U.S. Provisional Application Serial No. 60/278,688 entitled “Dual Bead Assays Using Cleavable Spacers to Improve Specificity and Sensitivity” filed Mar. 26, 2001, which is herein incorporated in its entirety by reference.

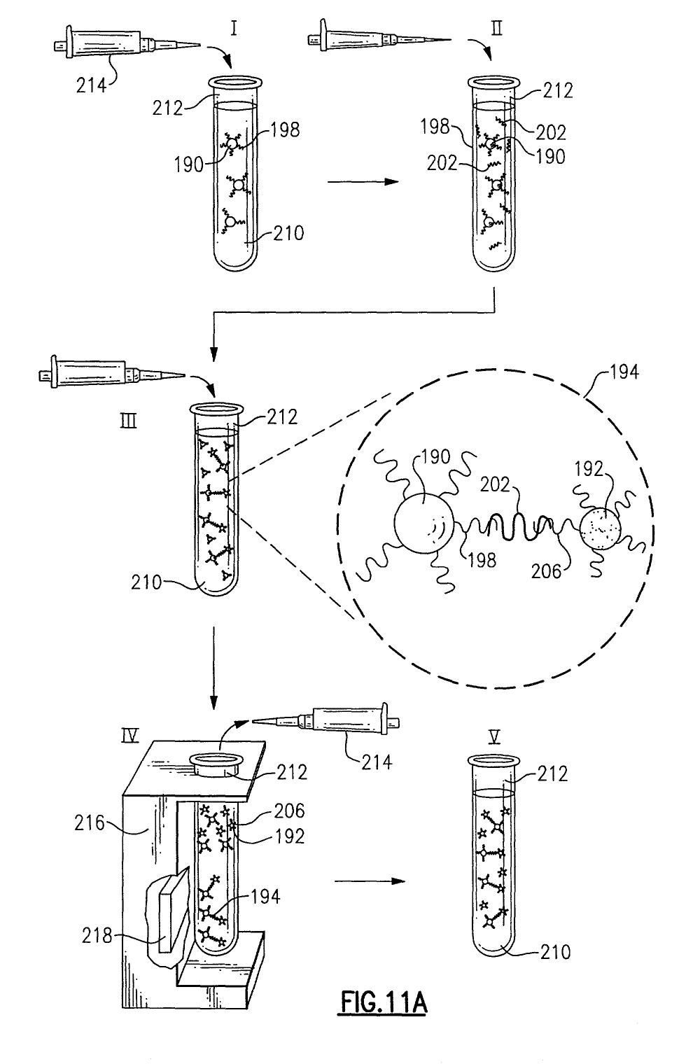

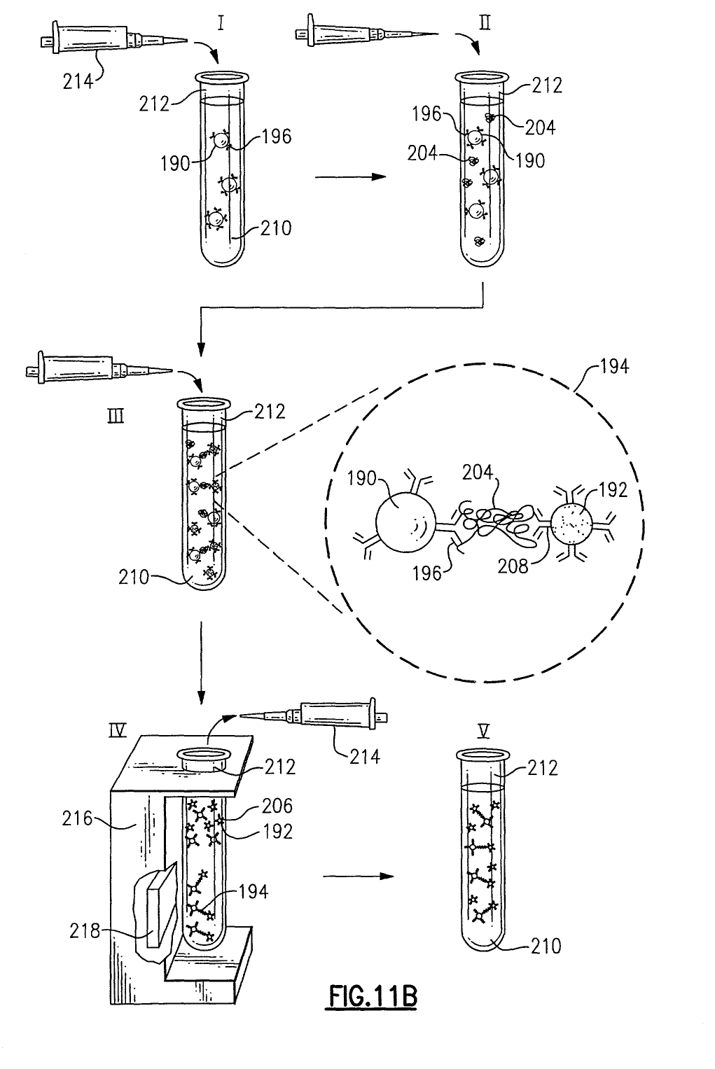

With reference now to FIG. 11A, there is illustrated a method of preparing a molecular assay using a “single-step hybridization” technique to create dual bead complex structures in a solution according to one aspect of the present invention. This method includes 5 principal steps identified consecutively as Steps I, II, III, IV, and V.

In Step I of this method, a number of capture beads 190 coated with oligonucleotide transport probes 198 are deposited into a test tube 212 containing a buffer solution 210. The number of capture beads 190 used in this method may be, for example, on the order of 10E+07 and each on the order of 1 micron or greater in diameter. Capture beads 190 are suspended in hybridization solution and are loaded into the test tube 212 by injection with pipette 214. The preferred hybridization solution is composed of 0.2M NaCl, 10 mM MgCl₂, 1 mM EDTA, 50 mM Tris-HCl, pH 7.5, and 5×Denhart's mix. A desirable hybridization temperature is 37 degrees Celsius. In a preliminary step in this embodiment, transport probes 198 are conjugated to 3 micron magnetic capture beads 190 by EDC conjugation. Further details regarding conjugation methods are disclosed in commonly assigned U.S. Provisional Application Serial No. 60/271,922 entitled, “Methods for Attaching Capture DNA and Reporter DNA to Solid Phase Including Selection of Bead Types as Solid Phase” filed Feb. 27, 2001; and U.S. Provisional Application Serial No. 60/277,854 entitled “Methods of Conjugation for Attaching Capture DNA and Reporter DNA to Solid Phase” filed Mar. 22, 2001, both of which are herein incorporated by reference in their entirety.

As shown in Step II, target DNA or RNA 202 is added to the solution. Oligonucleotide transport probes 198 are complementary to the DNA or RNA target agent 202. The target DNA or RNA 202 thus binds to the complementary sequences of transport probe 198 attached to the capture bead 190 as shown in FIG. 8A.

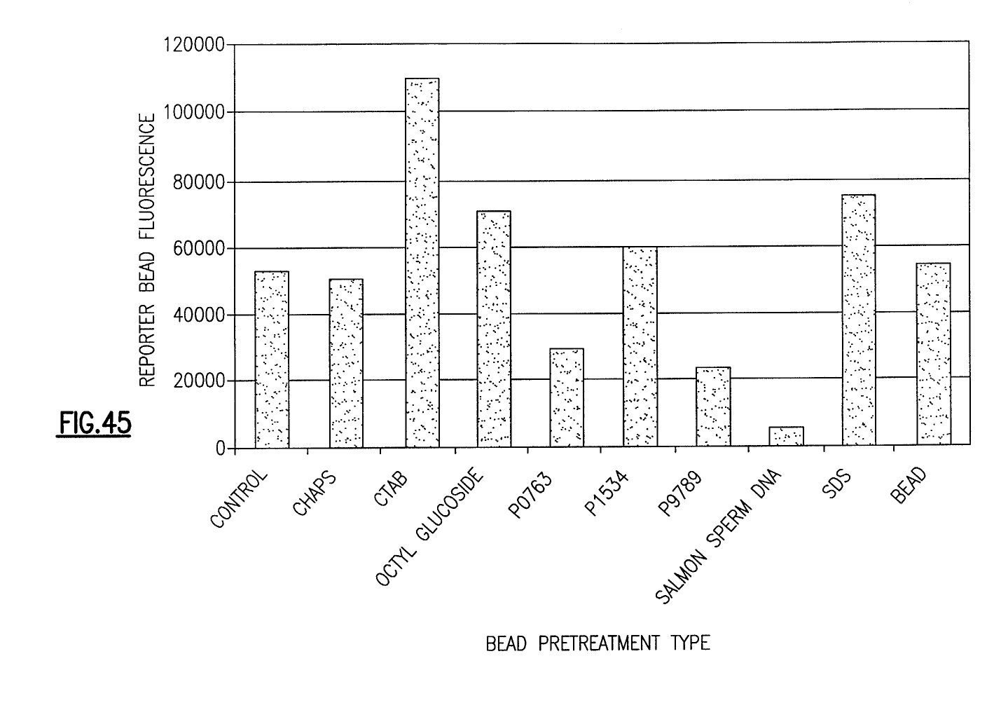

With reference now to Step III, there is added to the solution 210 reporter beads 192 coated with oligonucleotide signal probes 206. As also shown in FIGS. 9A and 10A, signal probes 206 are complementary to the target DNA or RNA 202. In one embodiment, signal probes 206, which are complementary to a portion of the target DNA or RNA 202, are conjugated to 2.1 micron fluorescent reporter beads 192. Signal probes 206 and transport probes 198 each have sequences that are complementary to the target DNA 202, but not complementary to each other. After adding reporter beads 192, the dual bead complex 194 is formed such that the target DNA 202 links capture bead 190 and reporter beads 192. With specific and thorough washing, there should be minimal non-specific binding between reporter bead 192 and capture bead 190. In addition to the washing step, non-specific bead binding may also be reduced by using blocking agents as discussed in FIG. 45 below. The target agent 202 and signal probe 206 are preferably allowed to hybridize for three to four hours at 37 degrees Celsius.

In this embodiment and others, it was found that intermittent mixing (i.e., periodically mixing and then stopping) produced greater yield of dual bead complex than continuous mixing during hybridization.

As next shown in Step IV, after hybridization, the dual bead complex 194 is separated from unbound reporter beads in the solution. The solution can be exposed to a magnetic field to capture the dual bead complex structures 194 using the magnetic properties of capture bead 190. The magnetic field can be encapsulated in a magnetic test tube rack 216 with a built-in magnet 218, which can be permanent or electromagnetic to draw out the magnetic beads and remove any unbound reporter beads in the suspension. Note that capture beads not bound to reporter beads will also be isolated.

The purification process illustrated in Step IV includes the removal of supernatant containing free-floating particles. Wash buffer is added into the test tube and the bead solution is mixed well. The preferred wash buffer for the one step assay consists of 145 mM NaCl, 50 mM Tris, pH 7.5, 0.1% SDS, 0.05% Tween, 0.25% NFDM, and 10 mM EDTA. Most of the unbound reporter beads 182, free-floating DNA, and non-specifically bound particles are agitated and removed from the supernatant. The dual bead complex can form a matrix of capture beads, target sequences, and reporter beads, wherein the wash process can further assist in the extraction of free floating particles trapped in the lattice structure of overlapping dual bead particles. Further details relating to other aspects associated with methods of decreasing non-specific binding of reporter beads to capture beads are disclosed in, for example, commonly assigned and co-pending U.S. Provisional Application Serial No. 60/272,134 entitled “Reduction of Non-Specific Binding in Dual Bead Assays by Selection of Bead Type and Bead Treatment” filed Feb. 28, 2001; and U.S. Provisional Application Serial No. 60/275,006 entitled “Reduction of Non-Specific Binding in Dual Bead Assays by Selection of Buffer Conditions and Wash Conditions” filed Mar. 12, 2001. Both of these applications are herein incorporated by reference in their entirety.

The last principal step shown in FIG. 11A is Step V. In this step, once the dual bead complex has been washed approximately 3-5 times with wash buffer solution, the assay mixture may be loaded into the disc and analyzed as illustrated in FIGS. 14, 25D and 26D below. Detection of the dual beads and reporter beads may also be carried out using a fluorescent detector provided that the reporter beads are fluorescent. Fluorescent detectors may include fluorescent optical disc readers, Fluorimagers, fluorescent microscopes, and fluorimeters. Data generated using a fluorimeter for fluorescent reporter bead detection in a dual bead assay are shown below in FIGS. 30A, 30B, 31, 42B, 43, and 45.

FIG. 11B illustrates an immunoassay using a “single-step antigen binding” method, similar to that in FIG. 11A, to create dual bead complex structures in a solution. This method similarly includes 5 principal steps. These steps are respectively identified as Steps I, II, III, IV, and V in FIG. 11A.

As shown in Step I, capture beads 190, e.g., on the order of 10E+07 in number and each on the order of 1 micron or above in diameter, which are coated with antibody transport probes 196 are added to a buffer solution 210. This solution may be that same as that employed in the method shown in FIG. 11A or alternatively may be specifically prepared for use with immunochemical assays. The antibody transport probes 196 have a specific affinity for the target antigen 204. The transport probes 196 bind specifically to epitopes within the target antigen 204 as also shown in FIG. 8B. In one embodiment, antibody transport probes 196 which have an affinity for a portion of the target antigen may be conjugated to 3 micron magnetic capture beads 190 via EDC conjugation. Alternatively, conjugation of the transport probes 196 to the capture bead 190 may be achieved by passive adsorption.

With reference now to Step II shown in FIG. 11B, the target antigen 204 is added to the solution. The target antigen 204 binds to the antibody transport probe 196 attached to the capture bead 190 as also shown in FIG. 8B.

As illustrated in Step III, reporter beads 192 coated with antibody signal probes 208 are added to the solution. Antibody signal probes 208 specifically binds to the epitopes on target antigen 204 as also represented in FIGS. 9B and 10B. In one embodiment, signal probes 208 are conjugated to 2.1 micron fluorescent reporter beads 192. Signal probes 208 and transport probes 196 each bind to specific epitopes on the target antigen, but not to each other. After adding reporter beads 192, the dual bead complex 194 is formed such that the target antigen 204 links capture bead 190 and reporter bead 192. With specific and thorough washing, there should be minimal non-specific binding between reporter bead 192 and capture bead 190. In addition to thorough washing, non-specific bead binding may also be reduced by using blocking agents as discussed below in FIG. 45.

In Step IV, after the binding in Step III, the dual bead complex 194 is separated from unbound reporter beads in the solution. The solution can be exposed to a magnetic field to capture the dual bead complex structures 194 using the magnetic properties of capture bead 190. The magnetic field can be encapsulated in a magnetic test tube rack 216 with a built-in magnet 218, which can be permanent or electromagnetic to draw out the magnetic beads and remove any unbound reporter beads in the suspension. Note that capture beads not bound to reporter beads will also be isolated.

The purification process of Step IV includes the removal of supernatant containing free-floating particles. Wash buffer is added into the test tube and the bead solution is mixed well. Most of the unbound reporter beads 182, free-floating protein samples, and non-specifically bound particles are agitated and removed from the supernatant. The dual bead complex can form a matrix of capture beads, target antigen, and reporter beads, wherein the wash process can further assist in the extraction of free floating particles trapped in the lattice structure of overlapping dual bead particles.

The last principal step in FIG. 11B is Step V. In this step, once the dual bead complex has been washed approximately 3-5 times with wash buffer solution, the assay mixture is loaded into the disc and is thereby in condition to be analyzed. Loading of the assay mixture into an optical bio-disc and bead detection using an optical disc reader is described in further detail in conjunction with FIGS. 14, 25A-25D and 26A-26D. A fluorecent detector may also be used to analyze fluorescent reporter beads in a dual bead assay. Fluorescent detectors may include fluorescent optical disc readers, Fluorimagers, fluorescent microscopes, and fluorimeters. Data generated using a fluorimeter to detect fluorescent reporter beads in a dual bead assay are shown below in FIGS. 30A, 30B, 31, 42B, 43, and 45.

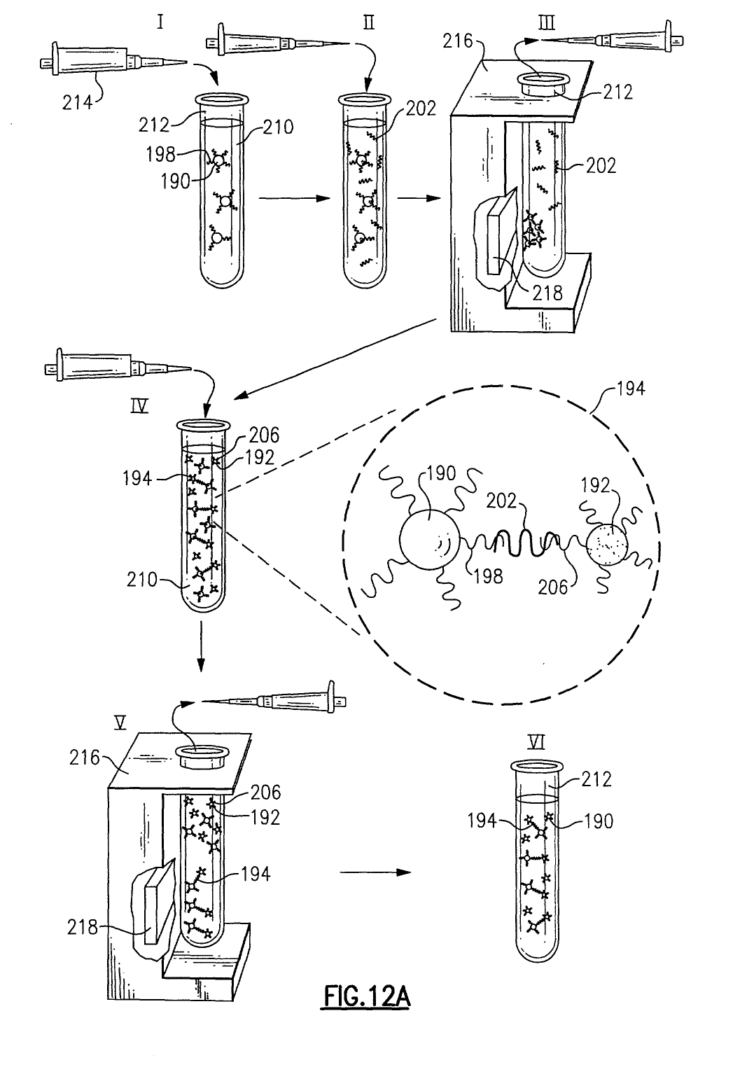

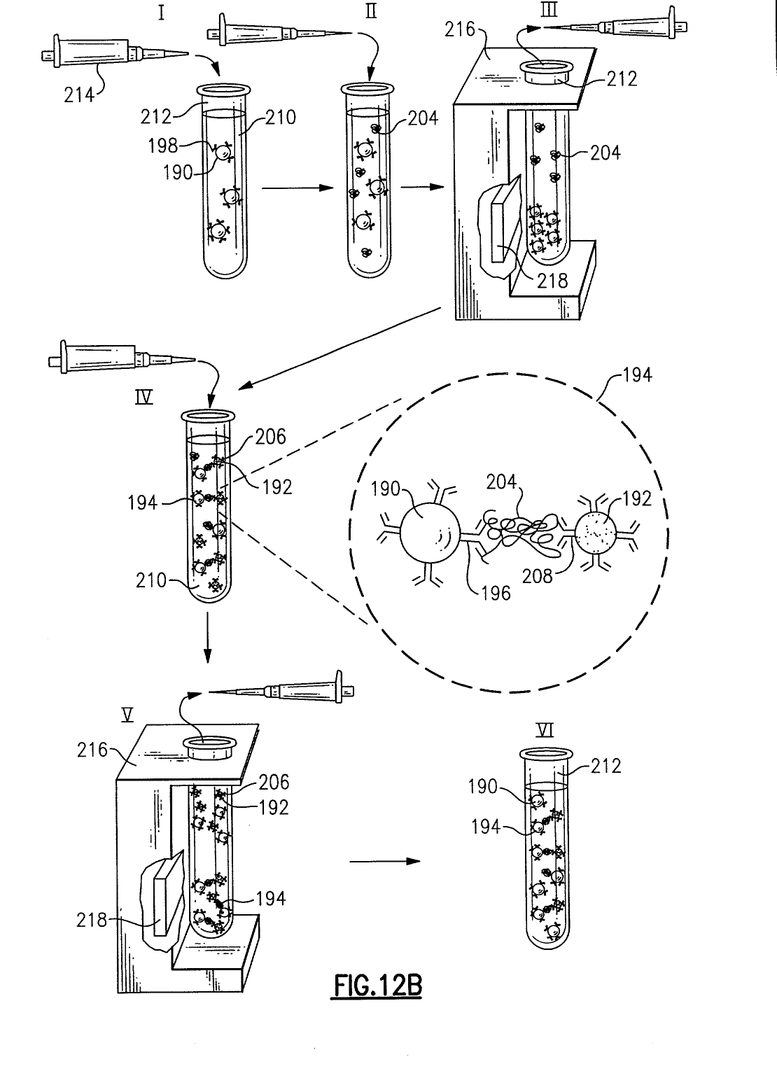

FIG. 12A shows an alternative genetic assay method referred to here as a “two-step hybridization” to create the dual bead complex which has 6 principal steps. Generally, capture beads are coated with oligonucleotide transport probes 198 complementary to DNA or RNA target agent and placed into a buffer solution. In this embodiment, transport probes which are complementary to a portion of target agent are conjugated to 3 micron magnetic capture beads via EDC conjugation. Other type of conjugation of the oligonucleotide transport probes to a solid phase may be utilized. These include, for example, passive adsorption or use of streptavidin-biotin interactions. These 6 main steps according to this method of the present invention are consecutively identified as Steps I, II, III, IV, V, and VI in FIG. 12A. The specific methodology used to perform the two-step hybridization in discussed in detail in Examples 1, 2, and 5.

More specifically now with reference to Step I shown in FIG. 12A, capture beads 190, suspended in hybridization solution, are loaded from the pipette 214 into the test tube 212. The preferred hybridization solution is composed of 0.2M NaCl, 10 mM MgCl₂, 1 mM EDTA, 50 mM Tris-HCl, pH 7.5, and 5×Denhart's mix. A desirable hybridization temperature is 37 degrees Celsius.

In Step II, target DNA or RNA 202 is added to the solution and binds to the complementary sequences of transport probe 198 attached to capture bead 190. In one specific embodiment of this method, target agent 202 and the transport probe 198 are allowed to hybridize for 2 to 3 hours at 37 degrees Celsius. Sufficient hybridization, however, may be achieved within 30 minutes at room temperature. At higher temperatures, hybridization may be achieved substantially instantaneously.

As next shown in Step III, target agents 202 bound to the capture beads are separated from unbound species in solution by exposing the solution to a magnetic field to isolate bound target sequences by using the magnetic properties of the capture bead 190. The magnetic field can be enclosed in a magnetic test tube rack 216 with a built-in magnet permanent 218 or electromagnet to draw out the magnetic beads and remove any unbound target DNA 202 free-floating in the suspension via pipette extraction of the solution. A wash buffer is added and the separation process can be repeated. The preferred wash buffer after the transport probes 198 and target DNA 202 hybridize, consists of 145 mM NaCl, 50 mM Tris, pH 7.5, and 0.05% Tween. Hybridization methods and techniques for decreasing non-specific binding of target agents to beads are further disclosed in commonly assigned and co-pending U.S. Provisional Application Serial No. 60/278,691 entitled “Reduction of Non-Specific Binding of Dual Bead Assays by Use of Blocking Agents” filed Mar. 26, 2001. This application is herein incorporated by reference in its entirety.

Referring now to Step IV illustrated in FIG. 12A, reporter beads 192 are added to the solution as discussed in conjunction with the method shown in FIG. 11A. Reporter beads 192 are coated with signal probes 206 that are complementary to target agent 202. In one particular embodiment of this method, signal probes 206, which are complementary to a portion of target agent 202, are conjugated to 2.1 micron fluorescent reporter beads 192. Signal probes 206 and transport probes 198 each have sequences that are complementary to target agent 202, but not complementary to each other. After the addition of reporter beads 192, the dual bead complex structures 190 are formed. As would be readily apparent to one of skill in the art, the dual bead complex structures are formed only if the target agent of interest is present. In this formation, target agent 202 links magnetic capture bead 190 and reporter bead 192. Using the preferred buffer solution, with specific and thorough washing, there is minimal non-specific binding between the reporter beads and the capture beads. In addition to the washing step, blocking agents may be used to reduce non-specific bead binding between capture and reporter beads as discussed in connection with FIG. 45 below. Target agent 202 and signal probe 206 are preferably allowed to hybridize for 2-3 hours at 37 degrees Celsius. As with Step II discussed above, sufficient hybridization may be achieved within 30 minutes at room temperature. At higher temperatures, the hybridization taking place in this step may also be achieved substantially instantaneously.

With reference now to Step V shown in FIG. 12A, after the hybridization in Step IV, the dual bead complex 194 is separated from unbound species in solution. The solution is again exposed to a magnetic field to isolate the dual bead complex 194 using the magnetic properties of the capture bead 190. Note again that the isolate will include capture beads not bound to reporter beads.

A purification process to remove supernatant containing free-floating particles includes adding wash buffer into the test tube and mixing the bead solution well. The preferred wash buffer for the two-step assay consists of 145 mM NaCl, 50 mM Tris, pH 7.5, 0.1% SDS, 0.05% Tween, 0.25% NFDM, and 10 mM EDTA. Most unbound reporter beads, free-floating DNA, and non-specifically bound particles are agitated and removed from the supernatant. The dual bead complex can form a matrix of capture beads, target agents, and reporter beads, wherein the wash process can further assist in the extraction of free floating particles trapped in the lattice structure of overlapping dual bead particles. Other related aspects directed to reduction of non-specific binding between reporter bead, target agent, and capture bead are disclosed in, for example, commonly assigned and co-pending U.S. Provisional Application Serial No. 60/272,243 entitled “Mixing Methods to Reduce Non-Specific Binding in Dual Bead Assays” filed Feb. 28, 2001; and U.S. Provisional Application Serial No. 60/272,485 entitled “Dual Bead Assays Including Linkers to Reduce Non-Specific Binding” filed Mar. 1, 2001, which are incorporated herein in their entirety.

The final principal step shown in FIG. 12A is Step VI. In this step, once the dual bead complex 194 has been washed approximately 3-5 times with wash buffer solution, the assay mixture is loaded into the disc and analyzed. Alternatively, during this step, the oligonucleotide signal and transport probes may be ligated to prevent breakdown of the dual bead complex during the disc analysis and signal detection processes. Further details regarding probe ligation methods are disclosed in commonly assigned and co-pending U.S. Provisional Application Serial No. 60/278,694 entitled “Improved Dual Bead Assays Using Ligation” filed Mar. 26, 2001, which is herein incorporated in its entirety by reference.

In accordance with another aspect of this invention, FIG. 12B shows an immuno-assay method, similar to those discussed in connection with FIGS. 11B and 12A, referred to here as a “two-step binding” to create the dual bead complex in an immunochemical assay. As with the method shown in FIG. 12A, this method includes 6 main steps. In general, capture beads coated with antibody transport probes which specifically binds to epitopes on target antigen are placed into a buffer solution. In one specific embodiment, antibody transport probes are conjugated to 3 micron magnetic capture beads. Different sized magnetic capture beads may be employed depending on the type of disc drive and disc assembly utilized to perform the assay. These 6 main steps according to this alternative method of the invention are respectively identified as Steps I, II, III, IV, V and VI in FIG. 12B.

With specific reference now to Step I shown in FIG. 12B, capture beads 190, suspended in buffer 210 solution, are loaded into a test tube 212 via injection from pipette 214.

In Step II, target antigen 204 is added to the solution and binds to the antibody transport probe 196 attached to capture bead 190. Target antigen 204 and the transport probe 196 are preferably allowed to bind for 2 to 3 hours at 37 degrees Celsius. Shorter binding times are also possible.

As shown in Step III, target antigen 204 bound to the capture beads 190 are separated from unbound species in solution by exposing the solution to a magnetic field to isolate bound target proteins or glycoproteins by using the magnetic properties of the capture bead 190. The magnetic field can be enclosed in a magnetic test tube rack 216 with a built-in magnet permanent 218 or electromagnet to draw out the magnetic beads and remove any unbound target antigen 204 free-floating in the suspension via pipette extraction of the solution. A wash buffer is added and the separation process can be repeated.

As next illustrated in Step IV, reporter beads 192 are added to the solution as discussed in conjunction with the method shown in FIG. 11B. Reporter beads 192 are coated with signal probes 208 that have an affinity for the target antigen 204. In one particular embodiment of this two-step immunochemical assay, signal probes 208, which bind specifically to a portion of target agent 204, are conjugated to 2.1 micron fluorescent reporter beads 192. Signal probes 208 and transport probes 196 each bind to specific epitopes on the target agent 204, but do not bind to each other. After the addition of reporter beads 192, the dual bead complex structures 190 are formed. As would be readily apparent to those skilled in the art, these dual bead complex structures are formed only if the target antigen of interest is present. In this formation, target antigen 204 links magnetic capture bead 190 and reporter bead 192. Using the preferred buffer solution, with specific and thorough washing, there is minimal non-specific binding between the reporter beads and the capture beads. Target antigen 204 and signal probe 208 are allowed to hybridize for 2-3 hours at 37 degrees Celsius. As with Step II discussed above, sufficient binding may be achieved within 30 minutes at room temperature. In the case of immunoassays temperatures higher than 37 degrees Celsius are not preferred because the proteins will denature.

Turning next to Step V as illustrated in FIG. 12B, after the binding shown in Step IV, the dual bead complex 194 is separated from unbound species in solution. This is achieved by exposing the solution to a magnetic field to isolate the dual bead complex 194 using the magnetic properties of the capture bead 190 as shown. Note again that the isolate will include capture beads not bound to reporter beads.

A purification process to remove supernatant containing free-floating particles includes adding wash buffer into the test tube and mixing the bead solution well. Most unbound reporter beads, free-floating proteins, and non-specifically bound particles are agitated and removed from the supernatant. The dual bead complex can form a matrix of capture beads, target agents, and reporter beads, wherein the wash process can further assist in the extraction of free floating particles trapped in the lattice structure of overlapping dual bead particles.

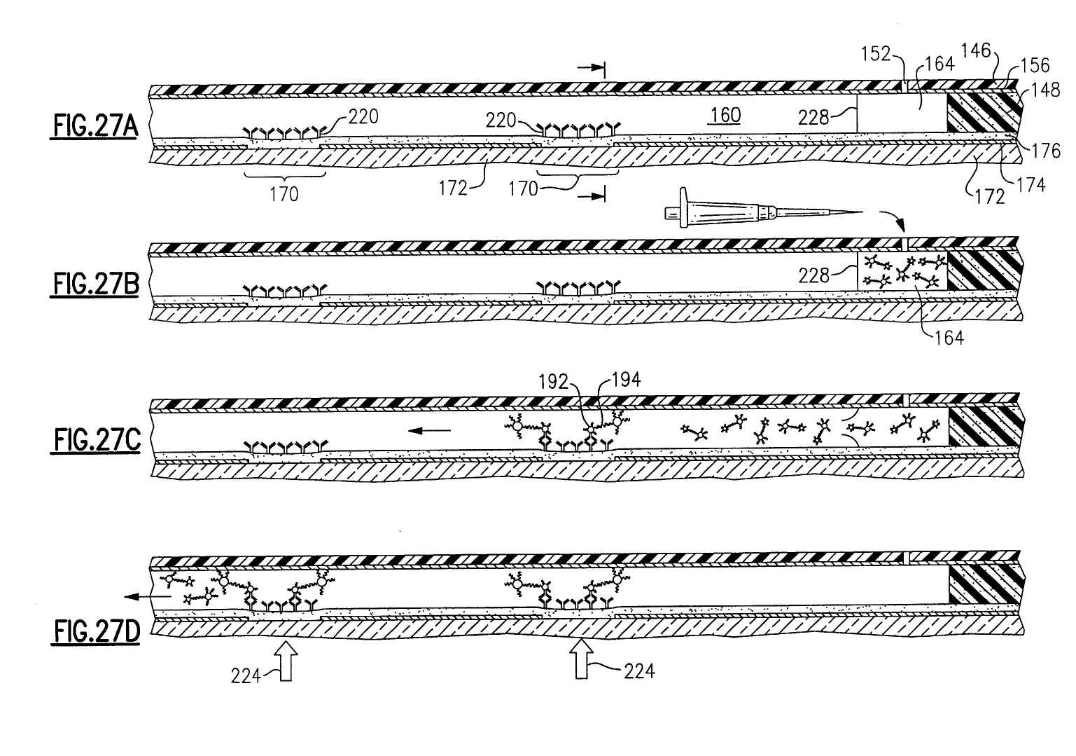

The final main step shown in FIG. 12B is Step VI. In this step, once the dual bead complex 194 has been washed approximately 3-5 times with wash buffer solution, the assay mixture is loaded into the disc and analyzed as described in further detail below with reference FIGS. 27A-27D. Fluorescent reporter beads in a dual bead test may also be carried out using a fluorescent type detector. Fluorescent detectors may include fluorescent optical disc readers, Fluorimagers, fluorescent microscopes, and fluorimeters. Data generated using a fluorimeter for fluorescent reporter bead detection in dual bead assays are illustrated below in FIGS. 30A, 30B, 31, 42B, 43, and 45.

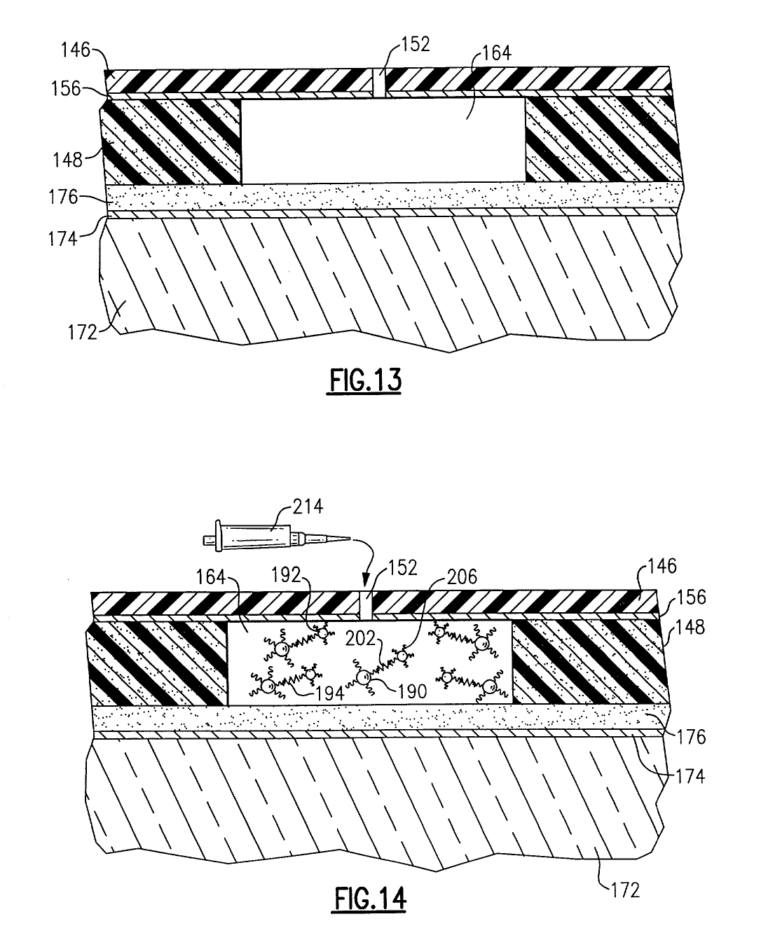

With reference now to FIG. 13, there is shown a cross sectional view illustrating the disk layers (similar to FIG. 6) of the mixing or loading chamber 164. Access to the loading chamber 164 is achieved by an inlet port 152 where the dual bead assay preparation is loaded into the disc system.

FIG. 14 is a view similar to FIG. 13 showing the mixing or loading chamber 164 with the pipette 214 injection of the dual bead complex 194 onto the disc. In this example, the complex includes reporters 192 and capture bead 190 linked together by the target DNA or RNA 202. The signal DNA 206 is illustrated as single stranded DNA complementary to the capture agent. The discs illustrated in FIGS. 13 and 14 may be readily adapted to other assays including the immunoassays and general molecular assays discussed above which employ, alternatively, proteins such as antigens or antibodies implemented as the transport probes, target agents, and signal probes accordingly.

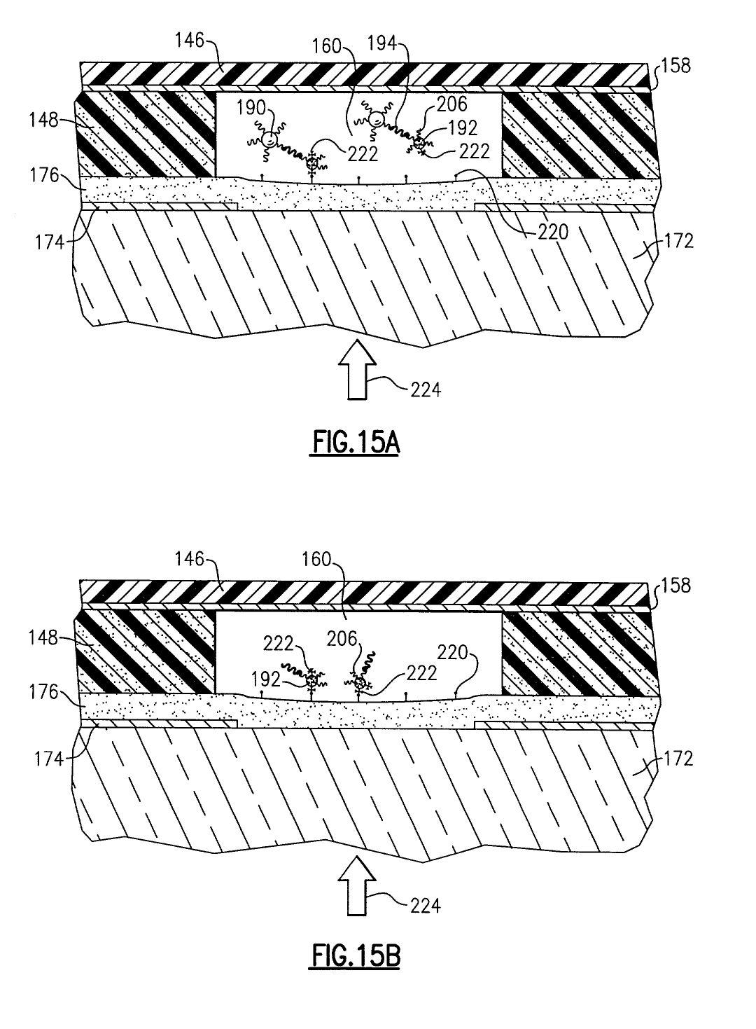

FIG. 15A shows the flow channel 160 and the target or capture zone 170 after anchoring of dual bead complex 194 to a capture agent 220. The capture agent 220 in this embodiment is attached to the active layer 176 by applying a small volume of capture agent solution to the active layer 176 to form clusters of capture agents within the area of the target zone 170. In this embodiment, the capture agent includes biotin or BSA-biotin. FIG. 15A also shows reporters 192 and capture beads 190 as components of a dual bead complex 194 as employed in the present invention. In this embodiment, anchor agents 222 are attached to the reporter beads 192. The anchor agent 222, in this embodiment, may include Sterptavidin or Neutravidin. So when the reporter beads 192 come in close proximity to the capture agents 220, binding occurs between the anchor probe 222/206 and the capture agent 220, via biotin-streptavidin interactions, thereby retaining the dual bead complex 194 within the target zone 170. At this point, an interrogation beam 224 directed to the target zone 170 can be used to detect the dual bead complex 194 within the target zone 170.

FIG. 15B is a cross sectional view similar to FIG. 15A illustrating the entrapment of the reporter bead 192 within the target zone 170 after a subsequent change in disc rotational speed. The change in rotational speed removes the capture beads 190 from the dual bead complex 194, ultimately isolating the reporter bead 192 in the target zone 170 to be detected by the interrogation or read beam 224.

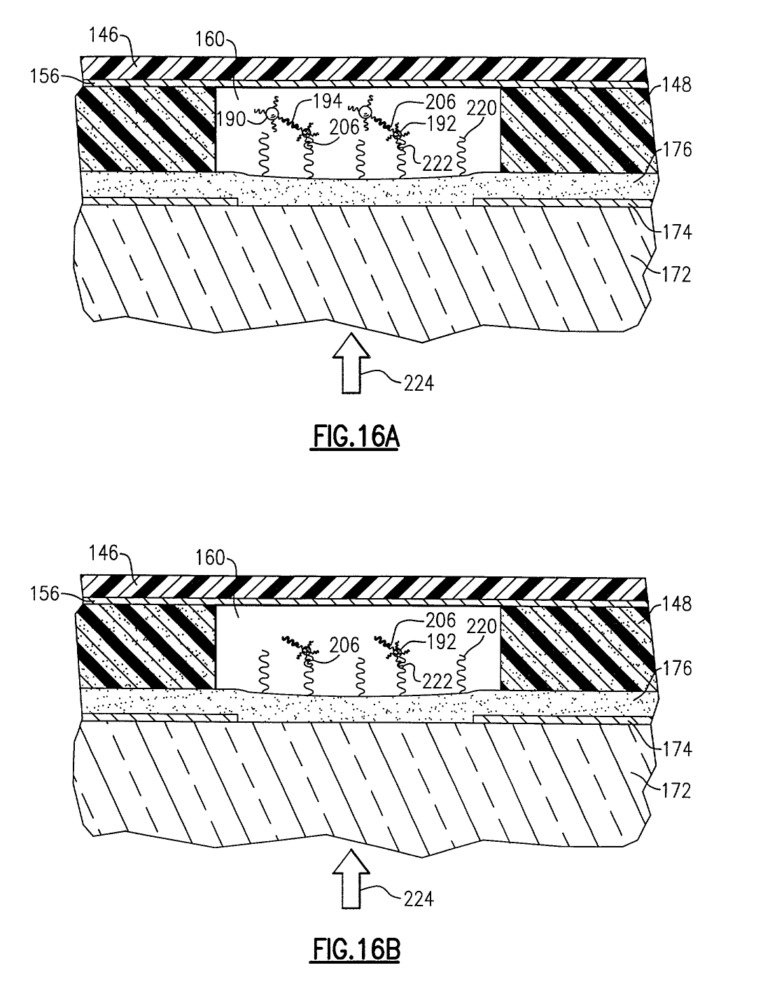

FIG. 16A is a cross sectional view, similar to FIG. 15A, that illustrates an alternative embodiment to FIG. 15A wherein the signal probes 206 or an anchor agent 222, on the reporter beads 192, directly hybridizes to the capture agent 220. FIG. 16A shows the flow channel 160 and the target or capture zone 170 after anchoring of dual bead complex 194 with the capture agent 220. The capture agent 220 in this embodiment is attached to the active layer 176 by applying a small volume of capture agent solution to the active layer 176 to form clusters of capture agents within the area of the target zone 170. Alternatively, the capture agent 220 may be attached to the active layer using an amino group that covalently binds to the active layer 176. In this embodiment, the capture agent includes DNA. FIG. 16A also shows reporters 192 and capture beads 190 as components of a dual bead complex 194 as employed in the present invention. In this embodiment, anchor probes 222 are attached to the reporter beads 192 The anchor agent 222, in this embodiment, may be a specific sequence of nucleic acids that are complimentary to the capture agent 220 or the oligonucleotide signal probe 206 itself. So when the reporter beads 192 come in close proximity to the capture agents 220, hybridization occurs between the anchor probe 222 and the capture agent 220 thereby retaining the dual bead complex 194 within the target zone 170. In an alternate embodiment, the signal probe 206 serves the function of anchor probe 222. At this point, an interrogation beam 224 directed to the target zone 170 may be used to detect the dual bead complex 194 within the target zone 170.

FIG. 16B illustrates the embodiment in FIG. 16A after a subsequent change in disc rotational speed. The change in rational speed removes the capture bead 190 from the dual bead complex 194, ultimately isolating the reporter bead 192 and the target DNA sequence 202 in the target zone 170 to be detected by an interrogation beam 224.

Referring now to FIG. 17, there is shown an alternative to the embodiment illustrated in FIG. 15A. In this embodiment, anchor agents 222 are attached to the capture beads 190 instead of the reporter beads. The anchor agent 222, in this embodiment, may include Streptavidin or Neutravidin. As in FIG. 15A, the target zone 170 is coated with a capture agent 220. The capture agent may include biotin or BSA-biotin. FIG. 17 also shows reporters 192 and capture beads 190 as components of a dual bead complex 194 as employed in the present invention. When the capture beads 190 come in close proximity to the capture agents 220, binding occurs between the anchor probe 222 and the capture agent 220, via biotin-streptavidin interactions, thereby retaining the dual bead complex 194 within the target zone 170. At this point, an interrogation beam 224 directed to the target zone 170 can be used to detect the dual bead complex 194 within the target zone 170.

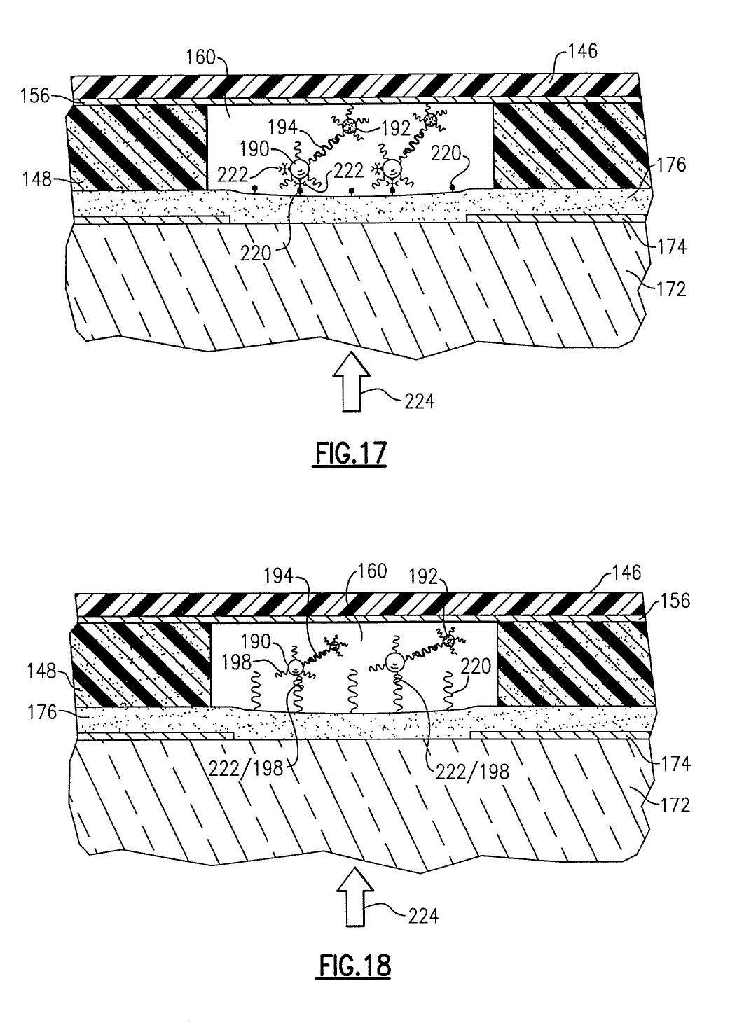

FIG. 18 is an alternative to the embodiment illustrated in FIG. 16A. In this embodiment, anchor agents 222 are attached to the capture beads 190 instead of the reporter beads. In this embodiment the transport probes 198, or an anchor agent 222 on the capture bead 190, directly hybridizes to the capture agent 220. In this embodiment, the capture agent 220 includes specific sequences of nucleic acid. The anchor agent 222, in this embodiment, may be a specific sequence of nucleic acids that are complimentary to the capture agent 220 or the oligonucleotide signal transport probe 198 itself. So when the capture beads 190 come in close proximity to the capture agents 220, hybridization occurs between the anchor agent 222 and the capture agent 220 thereby retaining the dual bead complex 194 within the target zone 170. At this point, an interrogation beam 224 directed to the target zone 170 can be used to detect the dual bead complex 194 within the target zone 170.

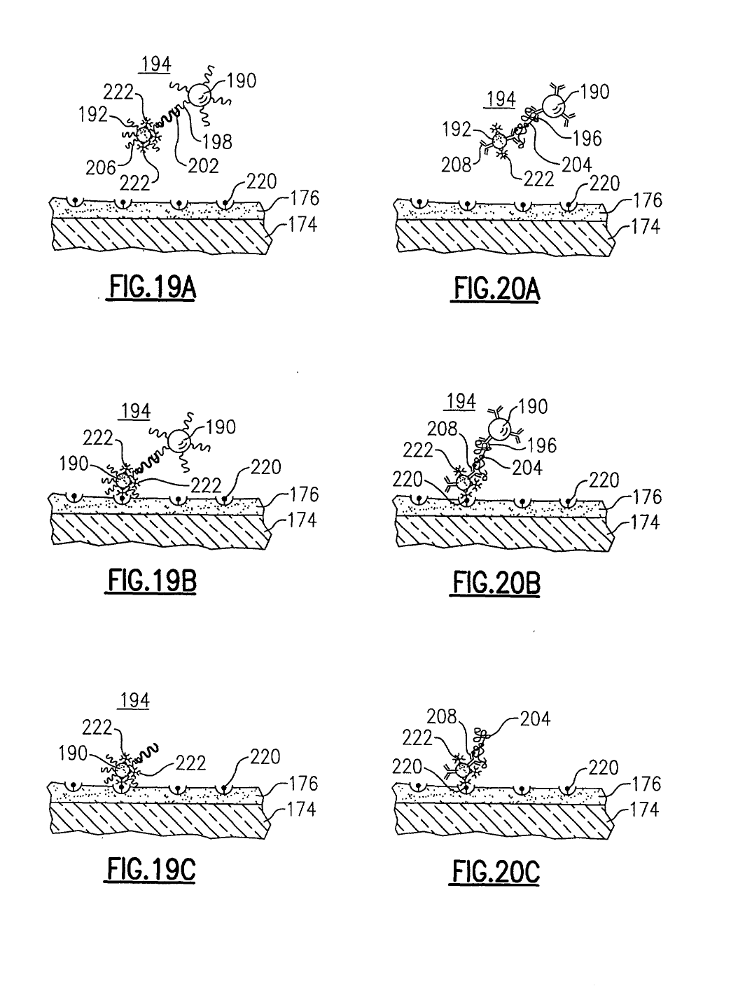

FIGS. 19A-19C are detailed partial cross sectional views showing the active layer 176 and the substrate 174 of the present bio-disc 110 as implemented in conjunction with the genetic assays discussed herein. FIGS. 19A-19C illustrates the capture agent 220 attached to the active layer 176 by applying a small volume of capture agent solution to the active layer 176 to form clusters of capture agents within the area of the target zone. The bond between capture agent 220 and the active layer 176 is sufficient so that the capture agent 220 remains attached to the active layer 176 within the target zone when the disc is rotated. FIGS. 19A and 19B also depict the capture bead 190 from the dual bead complex 194 binding to the capture agent 220 in the capture zone. These dual bead complexes are prepared according to the methods such as those discussed in FIGS. 11A and 12A. The capture agent 220 includes biotin and BSA-biotin. In this embodiment, the reporter bead 192 anchors the dual bead complex 194 in the target zone via biotin/streptavidin interactions. Alternatively, the target zone may be coated with streptavidin and may bind biotinylated reporter beads. FIG. 19C illustrates an alternative embodiment which includes an additional step to those discussed in connection with FIGS. 19A and 19B. In this preferred embodiment, a variance in the disc rotations per minute may create a centrifugal force enough to break the capture beads 190 away from the dual bead complex 194 based on the differential size and/or mass of the bead. Although there is a shift in the rotation speed of the disc, the reporter bead 192 remains anchored to the target zone. Thus, the reporter beads 192 are maintained within the target zone and detected using an optical bio-disc reader.

FIGS. 20A, 20B, and 20C illustrate an alternative embodiment to the embodiment discussed in FIGS. 19A-19C. FIGS. 20A-20C show detailed partial cross sectional views of a target zone implemented in conjunction with immunochemical assays. FIGS. 20A and 20B also depict the capture bead 190 from the dual bead complex 194 binding to the capture agent 220 in the capture zone. The capture agent 220 includes biotin and BSA-biotin. These dual bead complexes may be prepared according to methods such as those discussed in FIGS. 11B and 12B. In this embodiment, the reporter bead 192 anchors the dual bead complex 194 in the target zone via biotin/streptavidin interactions.

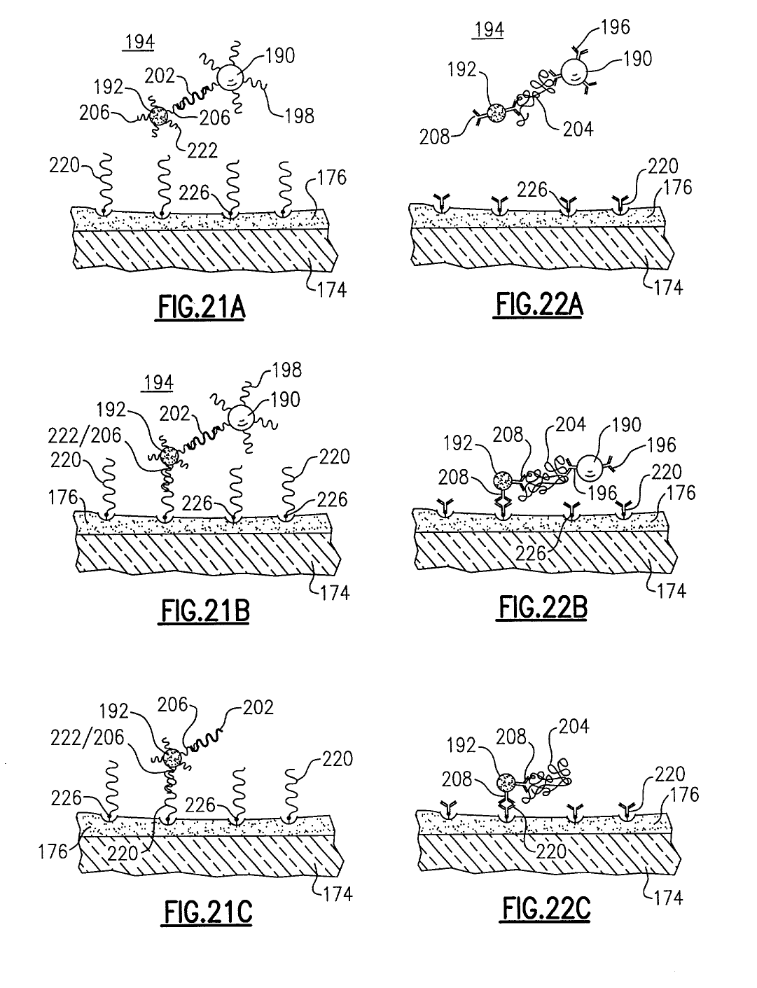

Referring now to FIGS. 21A, 21B, and 21C, there is shown detailed partial cross sectional views of a target zone including the active layer 176 and the substrate 174 of the present bio-disc 110 as implemented in conjunction with the genetic assays discussed herein. FIGS. 21A-21C illustrate the capture agent 220 attached to the active layer 176 by use of an amino group 226 which is made an integral part of the capture agent 220. As indicated, the capture agent 220 is situated within the target zone. The bond between the amino group 226 and the capture agent 220, and the amino group 226 and the active layer 176 is sufficient so that the capture agent 220 remains attached to the active layer 176 within the target zone when the disc is rotated. The preferred amino group 226 is NH₂. A thiol group may alternatively be employed in place of the amino group 226. In this embodiment of the present invention, the capture agent 220 includes the specific sequences of amino acids that are complimentary to the anchor agent 222 or oligonucleotide signal probe 206 which are attached to the reporter bead 192.

FIG. 21B depicts the reporter bead 192 of the dual bead complex 194, prepared according to methods such as those discussed in FIGS. 11A and 12A, binding to the capture agent 220 in the target zone. As the dual bead complex 194 flows towards the capture agent 220 and is in sufficient proximity thereto, hybridization occurs between the anchor agent 222 or oligonucleotide signal probe 206 and the capture agent 220. Thus, the reporter bead 192 anchors the dual bead complex 192 within the target zone.

FIG. 21C illustrates an alternative embodiment that includes an additional step to those discussed in connection with FIGS. 21A-21B. In this preferred embodiment, a variance in the disc rotations per minute may create enough centrifugal force to break the capture beads 190 away from the dual bead complex 194 based on the differential size and/or mass of the bead. Although there is a shift in the rotation speed of the disc, the reporter bead 192 with the target DNA sequence 202 remains anchored to the target zone. In either case, the reporter beads 192 are maintained within the target zone as desired.

FIGS. 22A, 22B, and 22C illustrate an alternative embodiment to the embodiment discussed in FIGS. 21A-21C. FIGS. 22A-22C show detailed partial cross sectional views of a target zone implemented in conjunction with immunochemical assays. FIGS. 22A and 22B also depict the reporter bead 192 from the dual bead complex 194, prepared according to methods such as those discussed in FIGS. 11B and 12B, binding to the capture agent 220 in the capture zone. In this embodiment, the capture agent 220 includes antibodies bound to the target zone by use of an amino group 226 which is made an integral part of the capture agent 220. Alternatively, the capture agents 220 may be bound to the active layer 176 by passive absorption, and hydrophobic or ionic interactions. In this embodiment, the reporter bead 192 anchors the dual bead complex 194 in the target zone via specific antibody binding. As with the embodiment illustrated in FIG. 21C, FIG. 22C shows an alternative embodiment that includes an additional step to those discussed in connection with FIGS. 22A-22B. In this alternative embodiment, a variance in the disc rotations per minute may create enough centrifugal force to break the capture beads 190 away from the dual bead complex 194 based on the differential size and/or mass of the bead. Although there is a shift in the rotation speed of the disc, the reporter bead 192 with the target antigen 204 remains anchored to the target zone. In either case, the reporter beads 192 are maintained within the target zone as desired.

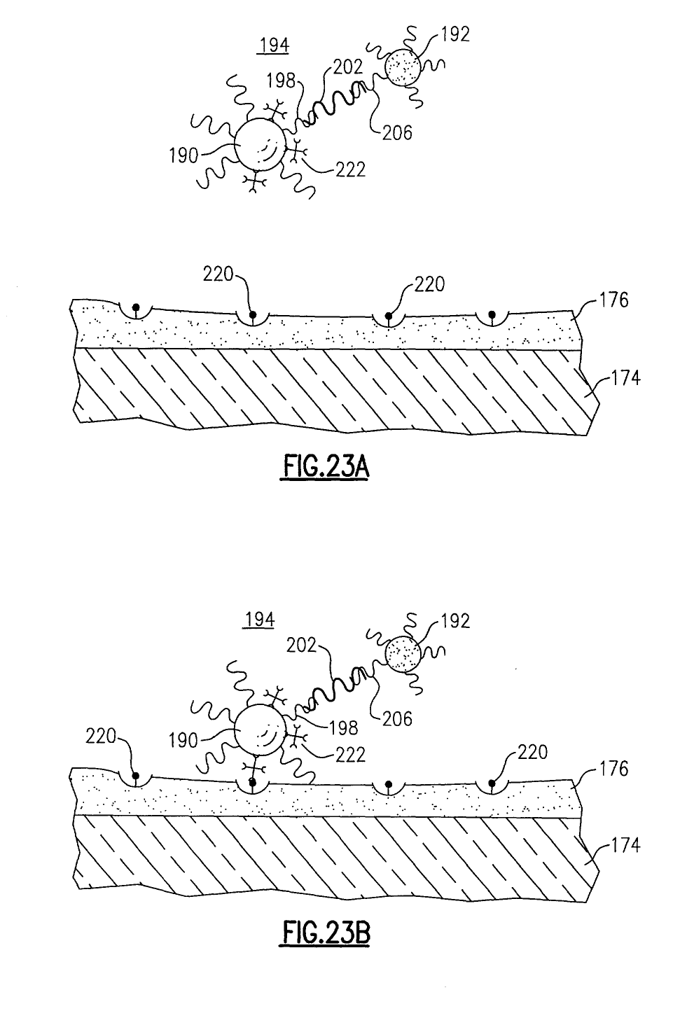

FIGS. 23A and 23B are detailed partial cross sectional views showing the active layer 176 and the substrate 174 of the present bio-disc 110 as implemented in conjunction with the genetic assays. FIGS. 23A and 23B illustrate an alternative embodiment to that discussed in FIGS. 19A and 19B above. In contrast to the embodiment in FIGS. 19A and 19B, in the present embodiment, the anchor agent 222 is attached to the capture bead 190 instead of the reporter bead 192. FIG. 23B illustrates the capture bead 190, from the dual bead complex 194, binding to the capture agent 220 in the capture zone. The capture agent 220 includes biotin and BSA-biotin. In this embodiment, the capture bead 190 anchors the dual bead complex 194 in the target zone via biotin/streptavidin interactions.

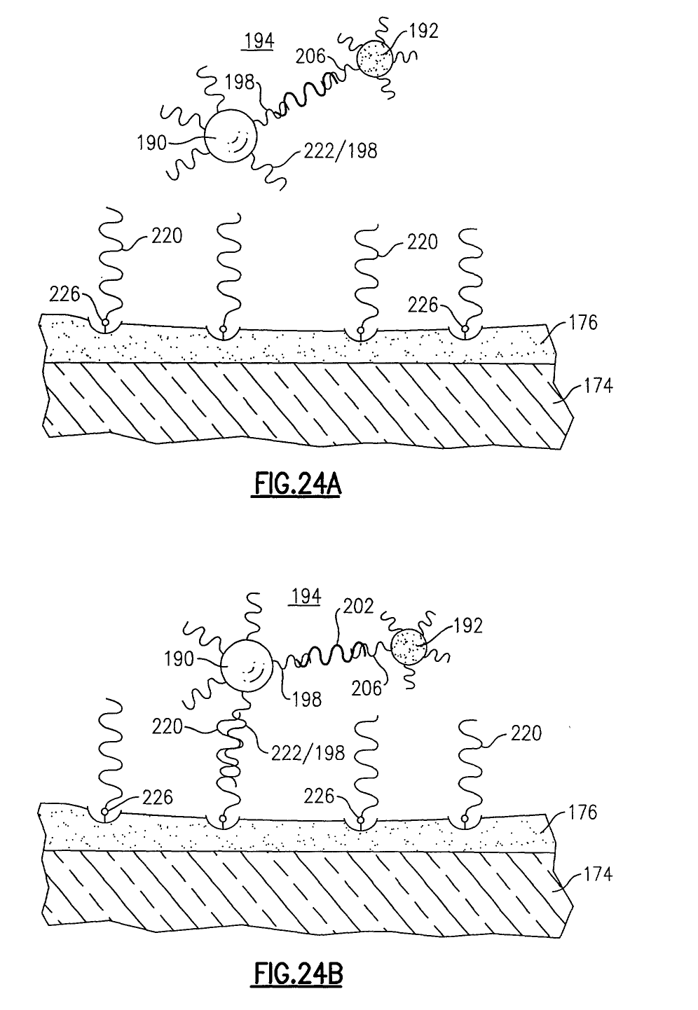

With reference now to FIGS. 24A and 24B, there is presented detailed partial cross sectional views showing the active layer 176 and the substrate 174 of the present bio-disc 110 as implemented in conjunction with the genetic assays. FIGS. 23A and 23B illustrate an alternative embodiment to that discussed in FIGS. 21A and 21B above. In contrast to the embodiment in FIGS. 21A and 21B, in the present embodiment, the anchor agent 222 is attached to the capture bead 190 instead of the reporter bead 192. FIG. 23B illustrates the capture bead 190, from the dual bead complex 194, binding to the capture agent 220 in the capture zone. The capture agent 220 is attached to the active layer 176 by use of an amino group 226 which is made an integral part of the capture agent 220. As indicated, the capture agent 220 is situated within the target zone. The bond between the amino group 226 and the capture agent 220, and the amino group 226 and the active layer 176 is sufficient so that the capture agent 220 remains attached to the active layer 176 within the target zone when the disc is rotated. In this embodiment of the present invention, the capture agent 220 includes the specific sequences of amino acids that are complimentary to the anchor agent 222 or oligonucleotide transport probe 198 which are attached to the capture bead 190. In this embodiment, the capture bead 190 anchors the dual bead complex 194 in the target zone via hybridization between the capture agent 220 and the anchor agent or the transport probe 198.

Disc Processing Methods

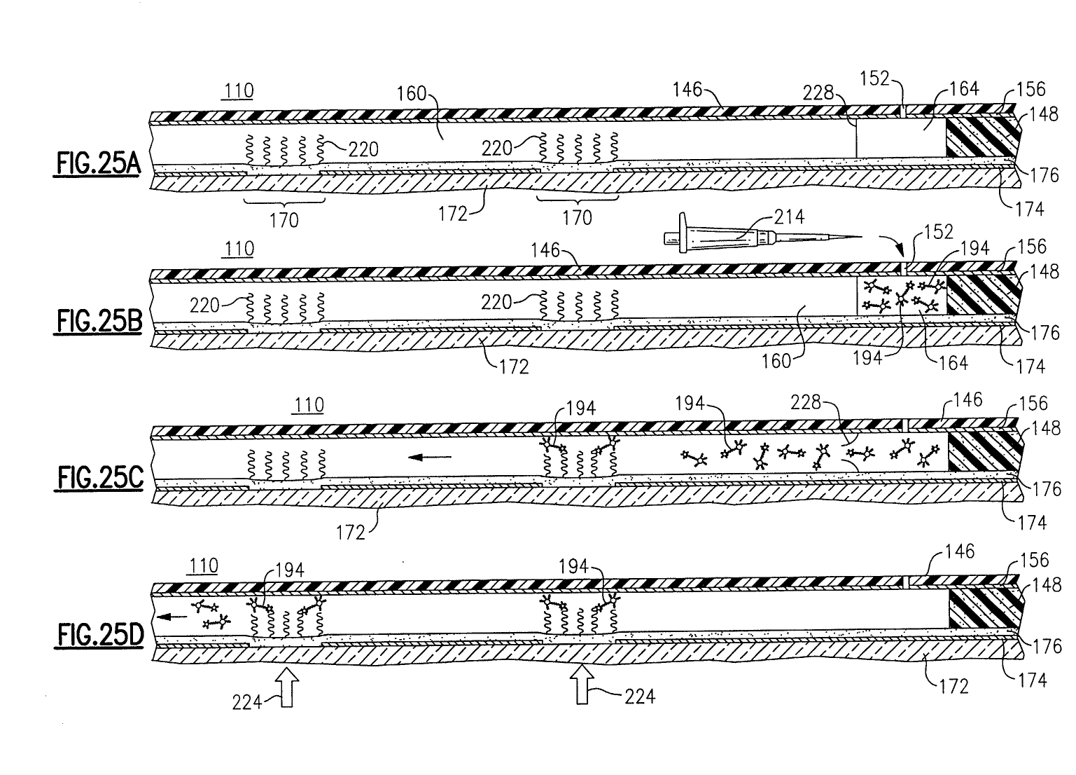

Turning now to FIGS. 25A-25D, there is shown the target zones 170 set out in FIGS. 21A-21C and FIGS. 24A-24B in the context of a disc, using as an input the solution created according to methods such as those shown in FIGS. 11A and 12A.

FIG. 25A shows a mixing/loading chamber 164, accessible through an inlet port 152, and leading to a flow channel 160. Flow channel 160 is pre-loaded with capture agents 220 situated in clusters in target zones 170. Each of the clusters of capture agents 220 is situated within a respective target zone 170. Each target zone 170 can have one type of capture agent or multiple types of capture agents, and separate target zones can have one and the same type of capture agent or multiple different capture agents in multiple capture fields. In the present embodiment, the capture agent can include specific sequences of nucleic acids that are complimentary to anchor agents 222 on either the reporter 192 or capture bead 190.

In FIG. 25B, a pipette 214 is loaded with a test sample of DNA or RNA that has been sequestered in the dual bead complex 194. The dual bead complex is injected into the flow channel 160 through inlet port 152. As flow channel 160 is further filled with the dual bead complex from pipette 214, the dual bead complex 194 begins to move down flow channel 160 as the disc is rotated. The loading chamber 164 can include a break-away retaining wall 228 so that complex 194 moves down the flow channel at one time.

In this embodiment, anchor agents 222, attached to reporter beads 192, bind to the capture agents 220 by hybridization, as illustrated in FIG. 25C. In this manner, reporter beads 192 are retained within target zone 170. Binding can be further facilitated by rotating the disc so that the dual bead complex 194 can slowly move or tumble down the flow channel. Slow movement allows ample time for additional hybridization. After hybridization, the disc can be rotated further at the same speed or faster to clear target zone 170 of any unattached dual bead complex 194, as illustrated in FIG. 25D.

An interrogation beam 224 can then be directed through target zones 170 to determine the presence of reporters, capture beads, and dual bead complex, as illustrated in FIG. 25D. In the event no target DNA or RNA is present in the test sample, there will be no dual bead complex structures, reporters, or capture beads bound to the target zones 170, but a small amount of background signal may be detected in the target zones from non-specific binding. In this case, when the interrogation beam 224 is directed into the target zone 170, a zero or low reading results, thereby indicating that no target DNA or RNA was present in the sample.

The speed, direction, and stages of rotation, such as one speed for one period followed by another speed for another period, can all be encoded in the operational information on the disc.

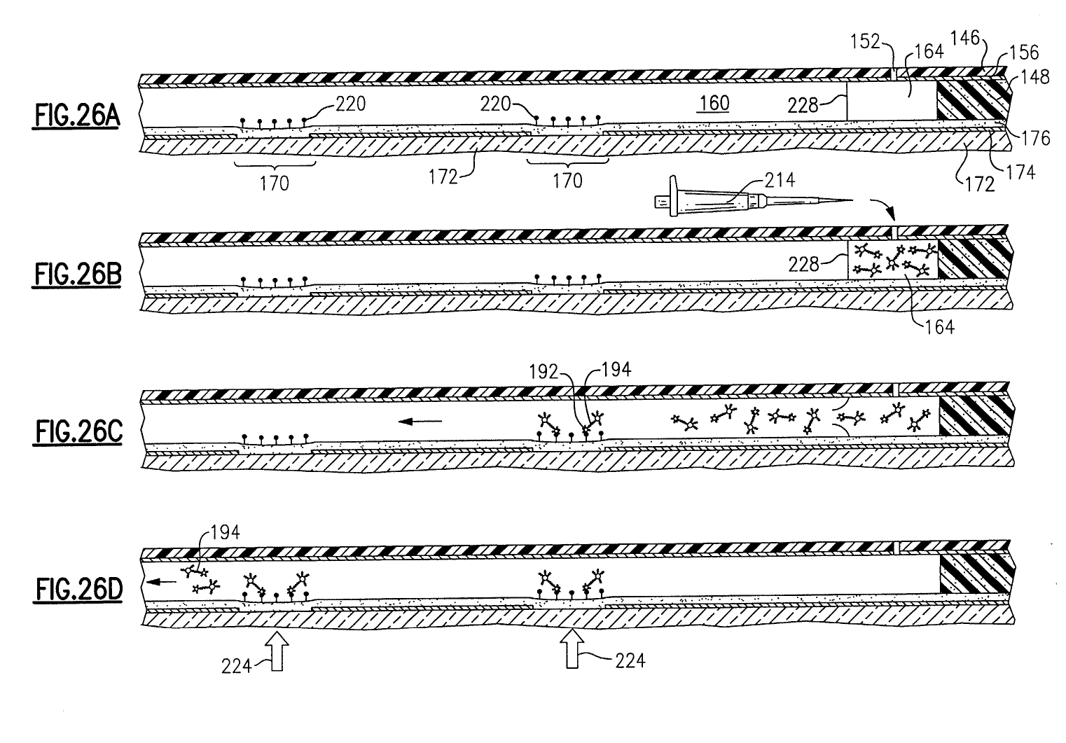

FIGS. 26A-26D show the target zones 170 including the capture chemistries discussed in FIGS. 19A-19C and FIGS. 23A-23B. This method uses as an input, the solution created according to methods shown in FIGS. 11A and 12A. FIGS. 26A-26D illustrate an alternative embodiment to that discussed in FIGS. 25A-25D showing a different bead capture method described in further detail below.

FIG. 26A shows a mixing/loading chamber 164, accessible through an inlet port 152, and leading to a flow channel 160. Flow channel 160 is pre-loaded with capture agents 220 situated in clusters in target zones 170. Each of the clusters of capture agents 220 is situated within a respective target zone 170. Each target zone 170 can have one type of capture agent or multiple types of capture agents, and separate target zones can have one and the same type of capture agent or multiple different capture agents in multiple capture fields. In the present embodiment, the capture agent can include specific biotin and BSA-biotin that has affinity to the anchor agents 222 on either the reporter 192 or capture bead 190. The anchor agents may include Streptavidin and Neutravidin.

In FIG. 26B, a pipette 214 is loaded with a test sample of DNA or RNA that has been sequestered in the dual bead complex 194. The dual bead complex is injected into the flow channel 160 through inlet port 152. As flow channel 160 is further filled with the dual bead complex from pipette 214, the dual bead complex 194 begins to move down flow channel 160 as the disc is rotated. The loading chamber 164 can include a break-away retaining wall 228 so that complex 194 moves down the flow channel at one time.

In this embodiment, anchor agents 222, attached to reporter beads 192, bind to the capture agents 220 by biotin-streptavidin interactions, as illustrated in FIG. 26C. In this manner, reporter beads 192 are retained within target zone 170. Binding can be further facilitated by rotating the disc so that the dual bead complex 194 can slowly move or tumble down the flow channel. Slow movement allows ample time for additional binding between the capture agent 220 and the anchor agent 222. After binding, the disc can be rotated further at the same speed or faster to clear target zone 170 of any unattached dual bead complex 194, as illustrated in FIG. 26D.

An interrogation beam 224 can then be directed through target zones 170 to determine the presence of reporters, capture beads, and dual bead complex, as illustrated in FIG. 26D. In the event no target DNA is present in the test sample, there will be no dual bead complex structures beads bound to the target zones 170. A small amount of background signal may be detected in the target zones from non-specific binding. In this case, when the interrogation beam 224 is directed into the target zone 170, a zero or low reading results, thereby indicating that no target DNA or RNA was present in the sample.

The speed, direction, and stages of rotation, such as one speed for one period followed by another speed for another period, can all be encoded in the operational information on the disc.

Referring next to FIGS. 27A-27D the is shown a series of cross sectional side views illustrating the steps of yet another alternative method according to the present invention. FIGS. 27A-27D show the target zones 170 including the capture mechanisms discussed in connection with FIGS. 22A-22C. This method uses an input the solution created according to the preparation methods shown in FIGS. 11B and 12B. FIGS. 27A-27D illustrate an immunochemical assay and an alternative bead capture method.

FIG. 27A shows a mixing/loading chamber 164, accessible through an inlet port 152, and leading to a flow channel 160. Flow channel 160 is pre-loaded with capture agents 220 situated in clusters in target zones 170. Each of the clusters of capture agents 220 is situated within a respective target zone 170. Each target zone 170 can have one type of capture agent or multiple types of capture agents, and separate target zones can have one and the same type of capture agent or multiple different capture agents in multiple capture fields. In the present embodiment, the capture agent can include antibodies that specifically bind to epitopes on the anchor agents 222 on either the reporter 192 or capture bead 190. Alternatively, the capture agent can directly bind to epitopes on the target antigen 204 within the dual bead complex 194. The anchor agents can include the target antigen, antibody transport probe 196, the antibody signal probe 208, or any antigen, bound to either the reporter bead 192 or the capture bead 190, that has epitopes than can specifically bind to the capture agent 220.

In FIG. 27B, a pipette 214 is loaded with a test sample of target antigen that has been sequestered in the dual bead complex 194. The dual bead complex is injected into the flow channel 160 through inlet port 152. As flow channel 160 is further filled with the dual bead complex from pipette 214, the dual bead complex 194 begins to move down flow channel 160 as the disc is rotated. The loading chamber 164 may include a break-away retaining wall 228 so that complex 194 moves down the flow channel at one time.

In this embodiment, anchor agents 222, attached to reporter beads 192, bind to the capture agents 220 by antibody-antigen interactions, as illustrated in FIG. 27C. In this manner, reporter beads 192 are retained within target zone 170. Binding can be further facilitated by rotating the disc so that the dual bead complex 194 can slowly move or tumble down the flow channel. Slow movement allows ample time for additional binding between the capture agents 220 and the anchor agent 222. After binding, the disc can be rotated further at the same speed or faster to clear target zone 170 of any unattached dual bead complex 194, as illustrated in FIG. 27D.

An interrogation beam 224 can then be directed through target zones 170 to determine the presence of reporters, capture beads, and dual bead complex, as illustrated in FIG. 27D. In the event no target antigen is present in the test sample, there will be no dual bead complex structures, reporters, or capture beads bound to the target zones 170, but a small amount of background signal may be detected in the target zones from non-specific binding. In this case, when the interrogation beam 224 is directed into the target zone 170, a zero or low reading results, thereby indicating that no target was present in the sample.

The speed, direction, and stages of rotation, such as one speed for one period followed by another speed for another period, can all be encoded in the operational information on the disc.

The methods described in FIGS. 25A-25D, 26A-26D, and 27A-27D are implemented using the reflective disc system 144. It should be understood that these methods and any other bead or sphere detection may also be carried out using the transmissive disc embodiment 180, as described in FIGS. 4A-4C, 5B, and 6B. It should also be understood that the methods described in FIGS. 11A-11B, 12A-12B, 25A-25D, 26A-26D, and 27A-27D are not limited to creating the dual bead complexes outside of the optical bio-discs but may include embodiments that use “in-disc” or “on-disc” formation of the dual bead complexes. In these on-disc implementations the dual bead complex is formed within the fluidic circuits of the optical bio-disc 110. For example, the dual bead formation may be carried out in the loading or mixing chamber 164. In one embodiment, the beads and sample are added to the disc at the same time, or nearly the same time. Alternatively, the beads with the probes can be pre-loaded on the disc for future use with a sample so that only a sample needs to be added.

The beads would typically have a long shelf life, with less shelf life for the probes. The probes can be dried or lyophilized (freeze dried) to extend the period during which the probes can remain in the disc. With the probes dried, the sample essentially reconstitutes the probes and then mixes with the beads to produce dual bead complex structures can be performed.

In either case, the basic process for on disc processing includes: (1) inserting the sample into a disc with beads with probes; (2) causing the sample and the beads to mix on the disc; (3) isolating, such as by applying a magnetic field, to hold the dual bead complex and move the non-held beads away, such as to a region referred to here as a waste chamber; and (4) directing the dual bead complexes (and any other material not moved to the waste chamber) to the capture fields. The detection process can be the same as one of those described above, such as by event detection or fluorimetry.

Detection and Related Signal Processing Methods and Apparatus

The number of reporter beads bound in the capture field can be detected in a qualitative manner, and may also be quantified by the optical disc reader.

The test results of any of the test methods described above can be readily displayed on monitor 114 (FIG. 1). The disc according to the present invention preferably includes encoded software that is read to control the controller, the processor, and the analyzer as shown in FIG. 2. This interactive software is implemented to facilitate the methods described herein and the display of results.

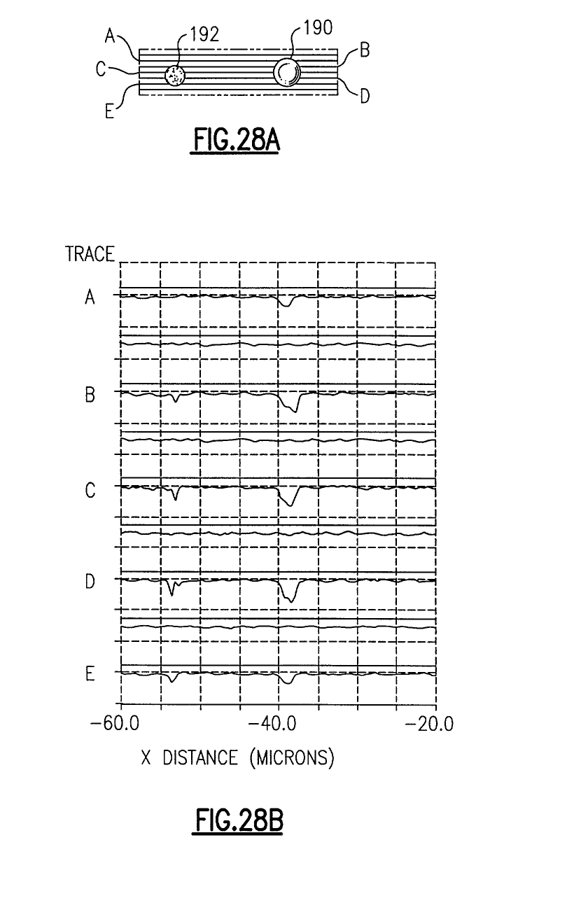

FIG. 28A is a graphical representation of an individual 2.1 micron reporter bead 192 and a 3 micron capture bead 190 positioned relative to the tracks A-E of an optical bio-disc according to the present invention.

FIG. 28B is a series of signature traces, from tracks A-E, derived from the beads of FIG. 28A utilizing a detected signal from the optical drive according to the present invention. These graphs represent the detected return beam 124. As shown, the signatures for a 2.1 micron reporter bead 190 are sufficiently different from those for a 3 micron capture bead 192 such that the two different types of beads can be detected and discriminated. A sufficient deflection of the trace signal from the detected return beam as it passes through a bead is referred to as an event.

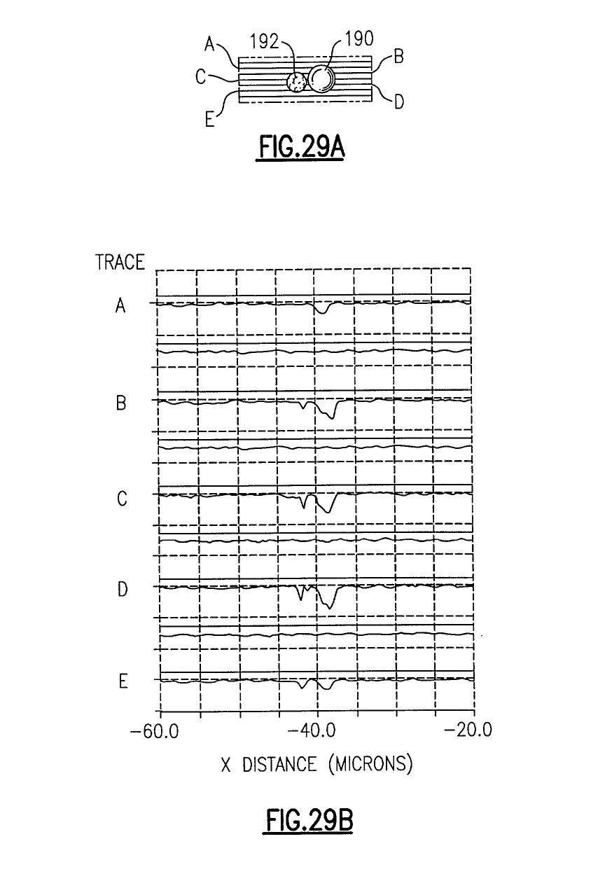

FIG. 29A is a graphical representation of a 2.1 micron reporter bead and a 3 micron capture bead linked together in a dual bead complex positioned relative to the tracks A-E of an optical bio-disc according to the present invention.

FIG. 29B is a series of signature traces, from tracks A-E, derived from the beads of FIG. 29A utilizing a detected signal from the optical drive according to the present invention. These graphs represent the detected return beam 124. As shown, the signatures for a 2.1 micron reporter bead 190 are sufficiently different from those for a 3 micron capture bead 192 such that the two different types of beads can be detected and discriminated. A sufficient deflection of the trace signal from the detected return beam as it passes through a bead is referred to as an event. The relative proximity of the events from the reporter and capture bead indicates the presence or absence the dual bead complex. As shown, the traces for the reporter and the capture bead are right next to each other indicating the beads are joined in a dual bead complex.

Alternatively, other detection methods can be used. For example, reporter beads can be fluorescent or phosphorescent. Detection of these reporters can be carried out in fluorescent or phosphorescent type optical disc readers. Other signal detection methods are described, for example, in commonly assigned co-pending U.S. patent application Ser. No. 10/008,156 entitled “Disc Drive System and Methods for Use with Bio-Discs” filed Nov. 9, 2001, which is expressly incorporated by reference; U.S. Provisional Application Serial Nos. 60/270,095 filed Feb. 20, 2001 and 60/292,108, filed May 18, 2001; and the above referenced U.S. patent application Ser. No. 10/043,688 entitled “Optical Disc Analysis System Including Related Methods For Biological and Medical Imaging” filed Jan. 10, 2002.

FIG. 30A is a bar graph of data generated using a fluorimeter showing a concentration dependent target detection using fluorescent reporter beads. This graph shows the molar concentration of target DNA versus number of detected beads. The dynamic range of target detection shown in the graph is 10E-16 to 10E-10 Molar (moles/liter). While the particular graph shown was generated using data from a fluorimeter, the results may also be generated using a fluorescent type optical disc drive.

FIG. 30B presents a standard curve demonstrating that the sensitivity of a fluorimeter is approximately 1000 beads in a fluorescent dual bead assay. The sensitivity of any assay depends on the sensitivity of the assay itself and on the sensitivity of the detection system. Referring to FIGS. 30A-30C, various studies were done to examine the sensitivity of the dual bead assay using different detection methods, e.g., a fluorimeter, and bio-disc detection according to the present invention.

As shown in FIG. 30B, the sensitivity of a fluorimeter is approximately 1000 beads in a fluorescent dual bead assay. In contrast, FIG. 30A shows that even at 10E-16 Molar (moles/liter), a sufficient number of beads over zero concentration can be detected to sense the presence of the target. With a sensitivity of 10E-16 Molar, a dual bead assay represents a very sensitive detection method for DNA that does not require DNA amplification (such as through PCR) and can be used to detect even a single bead.

In contrast to conventional detection methods, the use of a bio-disc coupled with a CD-reader (FIG. 1) improves the sensitivity of detection. For example, while detection with a fluorimeter is limited to approximately 1000 beads (FIG. 30B), use of a bio-disc coupled with CD-reader may enable the user to detect a single bead with the interrogation beam (FIG. 30C). Thus, the bioassay system provided herein improves the sensitivity of dual bead assays significantly. The detection of single beads using an optical bio-disc is discussed in detail in conjunction with FIGS. 28A and 28B above. FIG. 28B shows the signal traces of each bead as detected by the CD-reader. Dual bead complexes may also be identified by the CD-reader using the unique signature trace collected from the detection of a dual bead complex as shown in FIGS. 29A and 29B. Different optical bio-disc platforms may be used in conjunction with the CD-reader for detection of beads including the reflective and transmissive disc format illustrated in FIGS. 3C and 4C, respectively.

FIG. 30C is a pictorial demonstrating the formation of the dual bead complex linked together by the presence of the target in a genetic assay. Sensitivity to within one reporter molecule is possible with the present dual bead assay quantified with a bio-CD reader shown in FIGS. 1 and 2 above. Similarly, the dual bead complex formation may also be implemented in an immunochemical assay format as illustrated above in FIGS. 7B, 8B, 9B, 10B, 11B, and 12B.

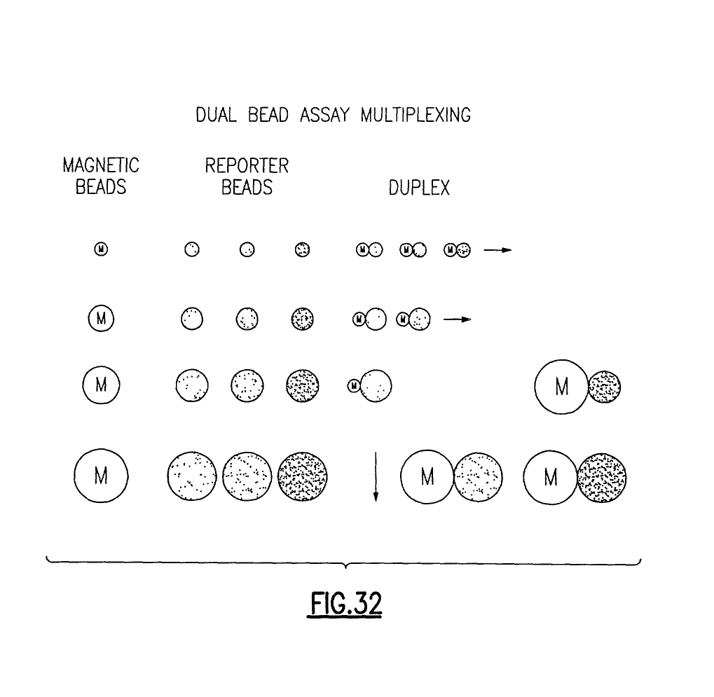

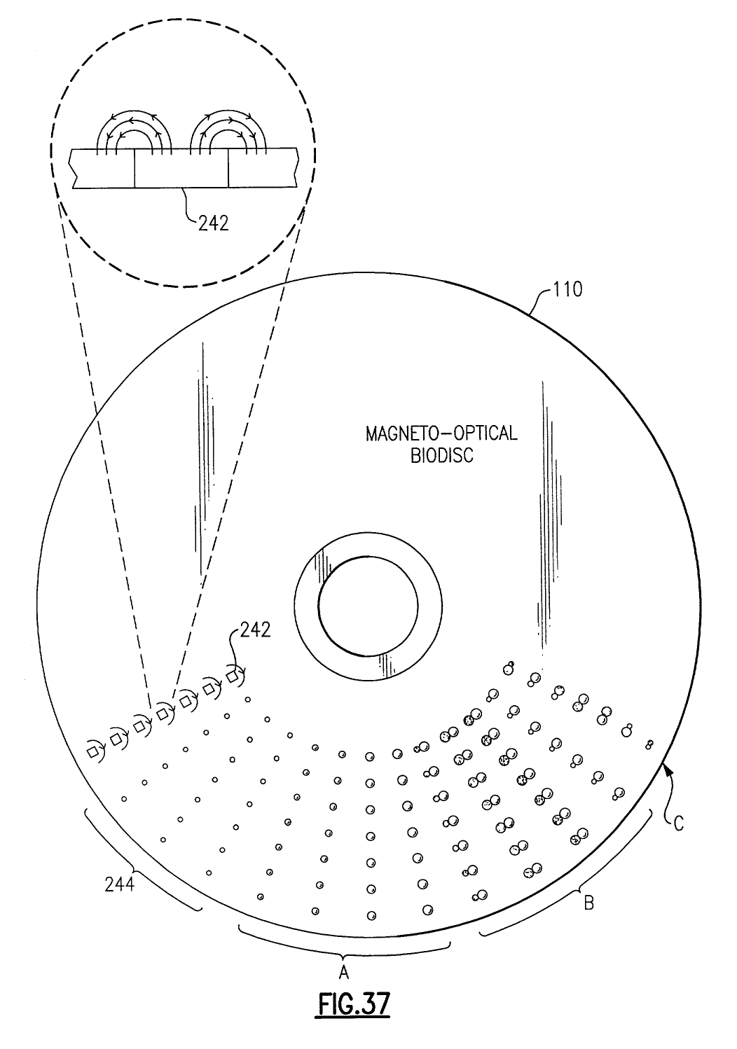

FIG. 31 shows data generated using a fluorimeter illustrating the concentration dependent detection of two different targets. The target detection was carried out in two different methods, the single and the duplex assays. In the single assay, the capture bead contains a transport probe specific to a single target and a reporter probe coated with a signal probe specific to the same target is mixed in a solution together with the target. In the duplex assay, the capture bead contains two different transport probes specific to two different targets. Mixing different reporter beads (red and green fluorescent or silica and polystyrene beads, for example) containing signal probes specific to one of the two targets, allows the detection of two different targets simultaneously. Detection of the dual bead duplex assay may be carried out using a magneto optical disc system as described below. FIGS. 32 and 37, discussed in further detail below, illustrate the formation and binding of various dual bead complexes onto an optical disc which may be detected by an optical bio-disc drive (FIG. 2), a magneto-optical disc system, a fluorescent disc system, or any similar device. Unique signature traces of a dual bead complex collected from an optical disc reader are shown in FIG. 29B above. The traces from FIG. 29B further illustrate that different bead types can be detected by an optical disc reader since different type beads will show a different signature profile.

Multiplexing, Magneto-Optical, and Magnetic Discs Systems

The use of a dual bead assay in the capture of targets allows for the use in multiplexing assays. This type of multiplexing is achieved by combining different sizes of magnetic beads and different types and sizes of reporter beads, different target agents can be detected simultaneously. As indicated in FIG. 32, four sizes of magnetic capture beads, and four sizes of three types of reporter beads produce up to 48 different types of dual bead complex. In a multiplexing assay, probes specific to different targets are thus conjugated to capture beads and reporter beads having different physical and/or optical properties, such as fluorescence at different wavelengths, to allow for the detection of different target agents simultaneously from the same biological sample in the same assay. As indicated in FIGS. 16A, 16B, 17A, and 17B, small differences in size can be detected by detecting reflected or transmitted light.

Multiple dual bead complex structures to capture different target agents can be carried out on or off the disc. If off the disc, the dual bead suspension is loaded into a port on the disc. The port is sealed and the disc is rotated in the disc reader. During spinning, free (unbound) beads are spun off to a periphery of the disc. The reporter beads detecting various target agents are thus localized in capture fields. In this manner, the presence of a specific target agent can be detected, and the amount of a specific target agent can be quantified by the disc reader.

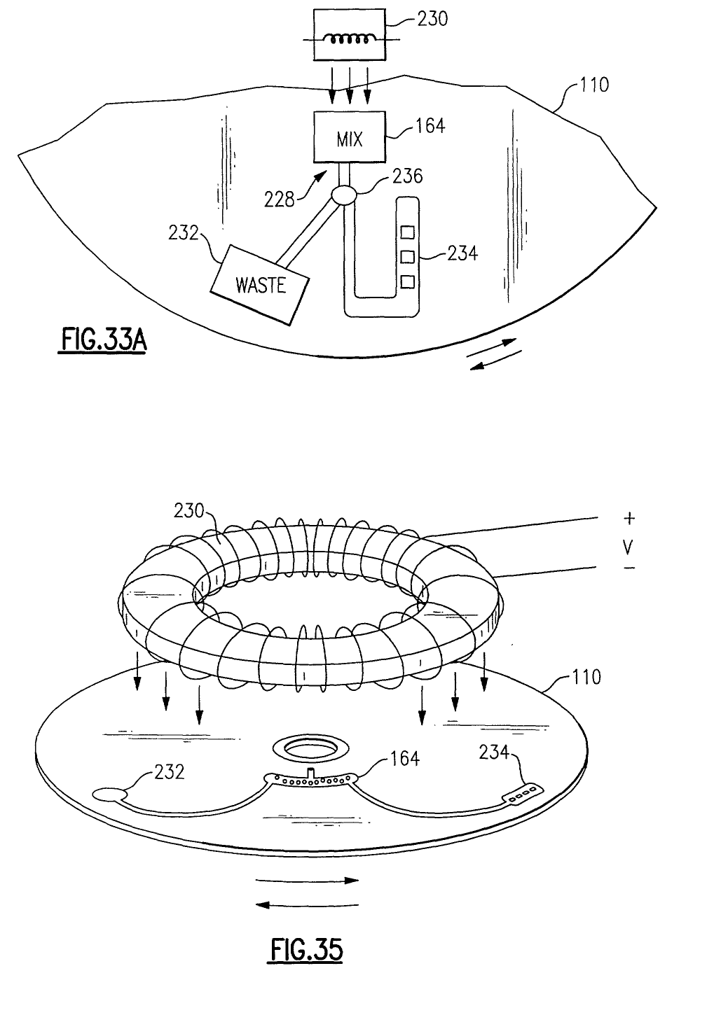

FIG. 33A is a general representation of an optical disc according to this aspect of the present invention and a method corresponding generally to the single-step method of FIGS. 11A and 11B is shown. The sample and beads can be added at one time or successively but closely in time, or the beads can be pre-loaded into a portion of the disc. These materials can be provided to a mixing chamber 164 that can have a breakaway wall 228 (see FIG. 25A) that holds in the solution within the mixing chamber 164. Mixing the sample and beads on the disc would be accomplished through rotation at a rate insufficient to cause the wall to break or the capillary forces to be overcome.

The disc can be rotated in one direction, or it can be rotated alternately in opposite directions to agitate the material in a mixing chamber. The mixing chamber is preferably sufficiently large so that circulation and mixing is possible. The mixing can be continuous or intermittent.

FIG. 33B shows one embodiment of a rotationally directionally dependent valve arrangement that is directionally dependent and uses a movable component for a valve. The mixing chamber leads to an intermediate chamber 244 that has a movable component, such as a ball 246. In the non-rotated state, the ball 246 may be kept in a slight recessed portion, or chamber 244 may have a gradual V-shaped tapering in the circumferential direction to keep the ball centered when there is no rotation.

Referring to FIGS. 33C and 33D in addition to FIGS. 33A and 33B, when the disc is rotated clockwise (FIG. 33C), ball 246 moves to a first valve seat 248 to block passage to detection chamber 234 and to allow flow to waste chamber 232, shown in FIG. 33A. When the disc is rotated counter-clockwise (FIG. 33D), ball 246 moves to a second valve seat 250 to block a passage to waste chamber 232 and to allow flow to detection chamber 234.

FIGS. 34A-34C show a variation of the prior embodiment in which the ball is replaced by a wedge 252 that moves one way or the other in response to acceleration of the disc. The wedge 252 can have a circular outer shape that conforms to the shape of an intermediate chamber 244. The wedge is preferably made of a heavy dense material relative to chamber 244 to avoid sticking. A coating can be used to promote sliding of the wedge relative to the chamber.

When the disc is initially rotated clockwise (FIG. 34B), the angular acceleration causes wedge 252 to move to block a passage to detection chamber 234 and to allow flow to waste chamber 232. When the disc is initially rotated counter-clockwise (FIG. 34C), the angular acceleration causes wedge 252 moves to block a passage to waste chamber 232 and allow flow to detection chamber 234. During constant rotation after the acceleration, wedge 252 remains in place blocking the appropriate passage.

In another embodiment of the present invention where the capture beads are magnetic, a magnetic field from a magnetic field generator or field coil 230 can be applied over the mixing chamber 164 to hold the dual bead complexes and unbound magnetic beads in place while material without magnetic beads are allowed to flow away to a waste chamber 232. This technique may also be employed to aid in mixing of the assay solution within the fluidic circuits or channels before any unwanted material is washed away. At this stage, only magnetic capture beads, unbound or as part of a dual bead complex, remain. The magnetic field is released, and the dual bead complex with the magnetic beads is directed to a capture and detection chamber 234.

The process of directing non-magnetic beads to waste chamber 232 and then magnetic beads to capture chamber 234 can be accomplished through the microfluidic construction and/or fluidic components. A flow control valve 236 or some other directing arrangement can be used to direct the sample and non-magnetic beads to waste chamber 232 and then to capture chamber 234. A number of embodiments for rotationally dependent flow can be used. Further details relating to the use of flow control mechanisms are disclosed in commonly assigned co-pending U.S. patent application Ser. No. 09/997,741 entitled “Dual Bead Assays Including Optical Biodiscs and Methods Relating Thereto” filed Nov. 27, 2001, which is herein incorporated by reference in its entirety.

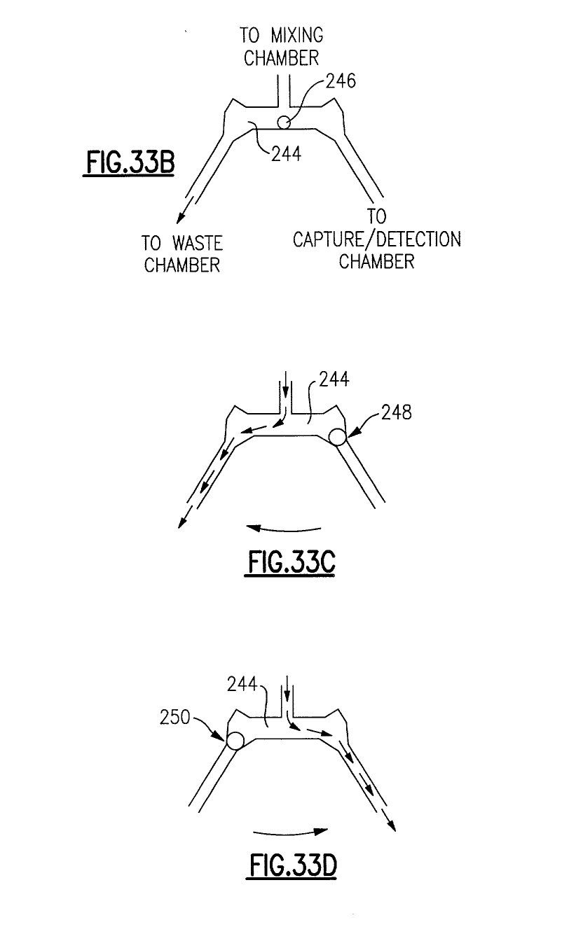

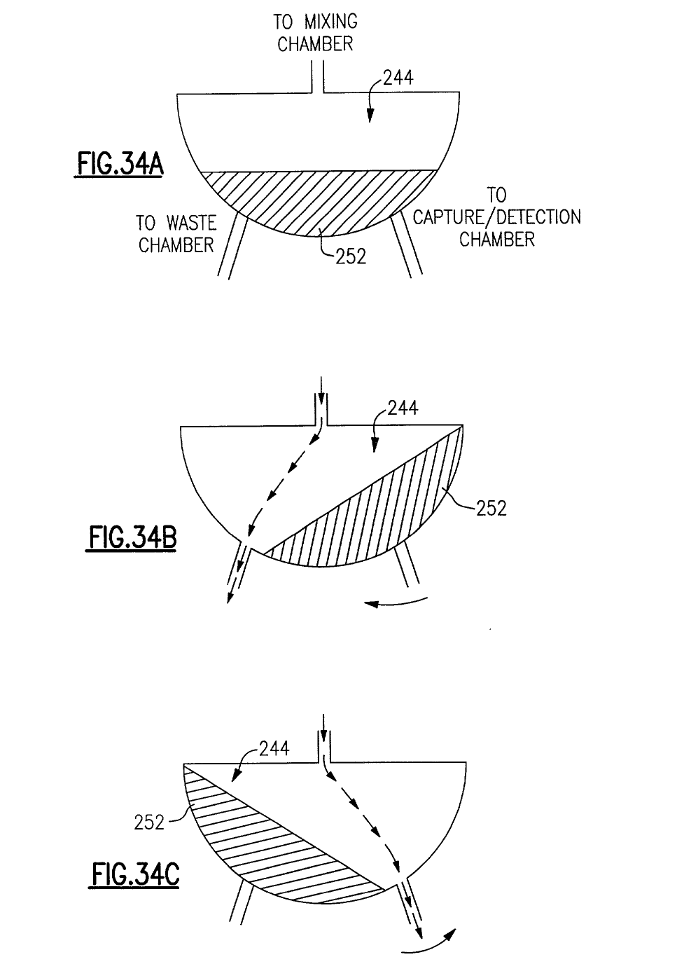

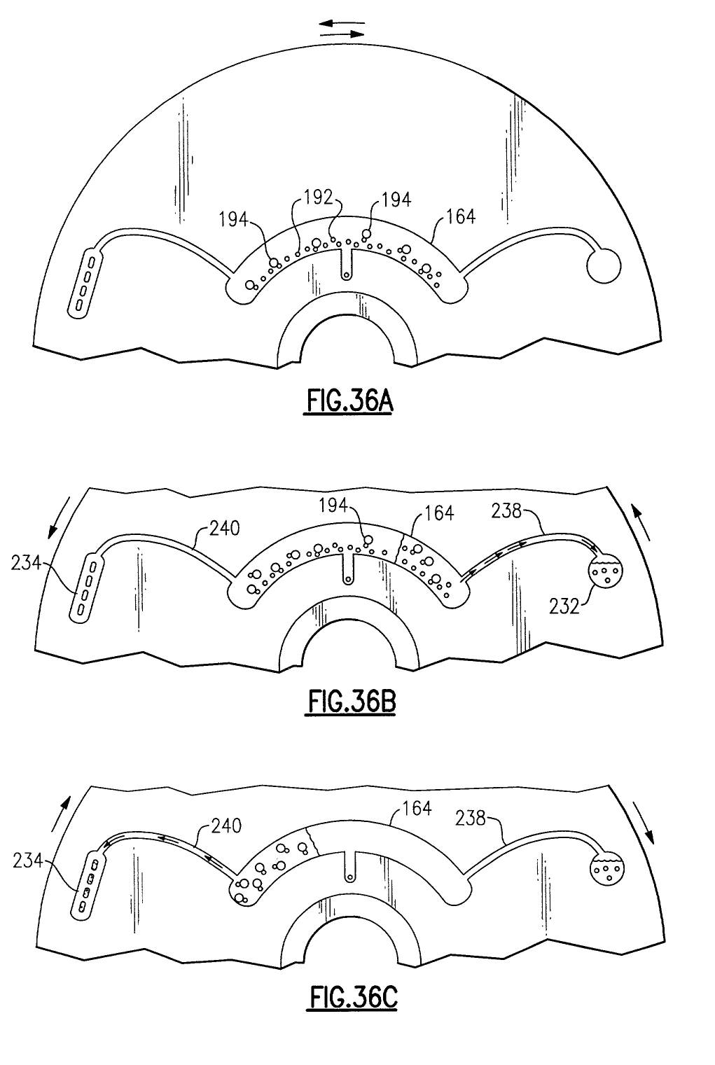

FIG. 35 is a perspective view of a disc including one embodiment of a fluidic circuit employed in conjunction with magnetic beads and the magnetic field generator 230 according to the present invention. FIG. 35 also shows the mixing chamber 164, the waste chamber 232, and the capture chamber 234. The magnetic field generator 230 is positioned over disc 110 and has a radius such that as disc 110 rotates, magnetic field generator 230 remains over mixing chamber 164, and is radially spaced from chambers 232 and 234. As with the prior embodiment discussed above, a magnetic field from the magnetic field generator 230 can be applied over the mixing chamber 164 to hold the dual bead complexes and/or unbound magnetic beads in place while additional material is allowed to enter the mixing chamber 164. The method of rotating the disc while holding magnetic beads in place with the magnetic field generator 230 may also be employed to aid in mixing of the assay solution within the mixing chamber 164 before the solution contained therein is directed elsewhere.

FIGS. 36A-36C are plan views illustrating a method of separation and detection for dual bead assays using the fluidic circuit shown in FIG. 35. FIG. 36A shows an unrotated optical disc with a mixing chamber 164 shaped as an annular sector holds a sample with dual bead complexes 194 and various unbound reporter beads 192. The electromagnet is activated and the disc is rotated counter-clockwise (FIG. 36B), or it can be agitated at a lower rpm, such as 1× or 3×. Dual bead complexes 194, with magnetic capture beads, remain in mixing chamber 164 while the liquid sample and the unbound reporter beads 192 move in response to angular acceleration to a rotationally trailing end of mixing chamber 164. The disc is rotated with sufficient speed to overcome capillary forces to allow the unbound reporter beads in the sample to move through a waste fluidic circuit 238 to waste chamber 232. At this stage in the process, the liquid will not move down the capture fluidic circuit 240 because of the physical configuration of the fluidic circuit as illustrated.

As illustrated next in FIG. 36C, the magnet is deactivated and the disc is rotated clockwise. Dual bead complexes 194 move to the opposite trailing end of the mixing chamber 164 in response to angular acceleration and then through a capture fluidic circuit 240 to the capture chamber 234. At this later stage in the process, the dual bead solution will not move down the waste fluidic circuit 238 due to the physical layout of the fluidic circuit, as shown. This embodiment shown in FIGS. 36A-36C thus illustrates directionally dependent flow as well as rotational speed dependent flow.

In this embodiment and others in which a fluidic circuit is formed in a region of the disc, a plurality of regions can be formed and distributed about the disc, for example, in a regular manner to promote balance. Furthermore, as discussed above, instructions for controlling the rotation can be provided on the disc. Accordingly, by reading the disc, the disc drive can have instructions to rotate for a particular period of time at a particular speed, stop for some period of time, and rotate in the opposite direction for another period of time. In addition, the encoded information can include control instructions such as those relating to, for example, the power and wavelength of the light source. Controlling such system parameters is particularly relevant when fluorescence is used as a detection method.

In yet another embodiment, a passage can have a material or configuration that can seal or dissolve either under influence from a laser in the disc drive, or with a catalyst pre-loaded in the disc, or such a catalyst provided in the test sample. For example, a gel may solidify in the presence of a material over time, in which case the time to close can be set sufficiently long to allow the unbound capture beads to flow to a waste chamber before the passage to the waste chamber closes. Alternatively, the passage to the waste chamber can be open while the passage to the detection chamber is closed. After the unbound beads are directed to the waste chamber, the passage to the direction chamber is opened by energy introduced from the laser to allow flow to the detection chamber.

FIG. 37 illustrates yet another embodiment of the optical disc 110 for use with a multiplexing dual bead assay. In this case, a disc, such as one used with a magneto-optical drive, has magnetic regions 242 that can be written and erased with a magnetic head. A magneto-optical disc drive, for example, can create magnetic regions 242 as small as 1 micron by 1 micron square. The close-up section of the magnetic region 242 shows the direction of the magnetic field with respect to adjacent regions.

The ability to write to small areas in a highly controllable manner to make them magnetic allows capture areas to be created in desired locations. These magnetic capture areas can be formed in any desired configuration or location in one chamber or in multiple chambers. These areas capture and hold magnetic beads when applied over the disc. The domains can be erased if desired, thereby allowing them to be made non-magnetic and allowing the beads to be released.

In one configuration of a magnetic bead array according to this aspect of the present invention, a set of three radially oriented magnetic capture regions 244 are shown, by way of example, with no beads attached to the magnetic capture regions in the columns. With continuing reference to FIG. 37, there is shown a set of four columns in Section A with individual magnetic beads magnetically attached to the magnetic areas in a magnetic capture region. Another set of four columns arrayed in Section B is shown after binding of reporter beads to form dual bead complexes attached to specific magnetic areas, with different columns having different types of reporter beads. As illustrated in Section B, some of the reporter beads utilized vary in size to thereby achieve the multiplexing aspects of the present invention as implemented on a magneto-optical biodisc. In Section C, a single column of various dual bead complexes is shown as another example of multiplexing assays employing various bead sizes individually attached at separate magnetic areas.

In a method for use with such a magneto-optical biodisc, the write head in an MO drive can be used to create magnetic areas, and then a sample can be directed over that area to capture magnetic beads provided in the sample. After introduction of the first sample set, other magnetic areas may also be created and another sample set can provided to the newly created magnetic capture region for detection. Thus detection of multiple sample sets may be performed on a single disc at different time periods. The magneto-optical drive also allows the demagnetization of the magnetic capture regions to thereby release and isolate the magnetic beads if desired. Thus this system provides for the controllable capture, detection, isolation, and release of one or more specific target molecules from a variety of different biochemical, chemical, or biological samples.

As described above, a sample can be provided to a chamber on a disc. Alternatively, a sample could be provided to multiple chambers that have sets of different beads. In addition, a series of chambers can be created such that a sample can be moved by rotational motion from one chamber to the next, and separate tests can then be performed in each chamber.

With such a disc, a large number of tests can be performed at one time and can be performed interactively. In this manner, when a test is performed and a result is obtained, the system can be instructed to create a new set of magnetic regions for capturing the dual bead complex. Regions can be created one at a time or in large groups, and can be performed in successive chambers that have different pre-loaded beads. Other processing advantages can be obtained with a disc that has writeable magnetic regions. For example, the “capture agent” is essentially the magnetic field created by in the magnetic region on the disc and therefore there is no need to add an additional biological or chemical capture agent.

Instructions for controlling the locations for magnetic regions written or erased on the disc, and other information such as rotational speeds, stages of rotation, waiting periods, wavelength of the light source, and other parameters can be encoded on and then read from the disc itself.

Methods for DNA Conjugation onto Solid Phase

Successful conjugation of probes to a solid phase such as a bead or a biodisc, is an important step for the dual bead assays of the invention. In certain embodiments of the invention, probes are attached covalently to the beads. Efficiency of the covalent conjugation depends on the type of bead utilized and the specific conjugation method employed.

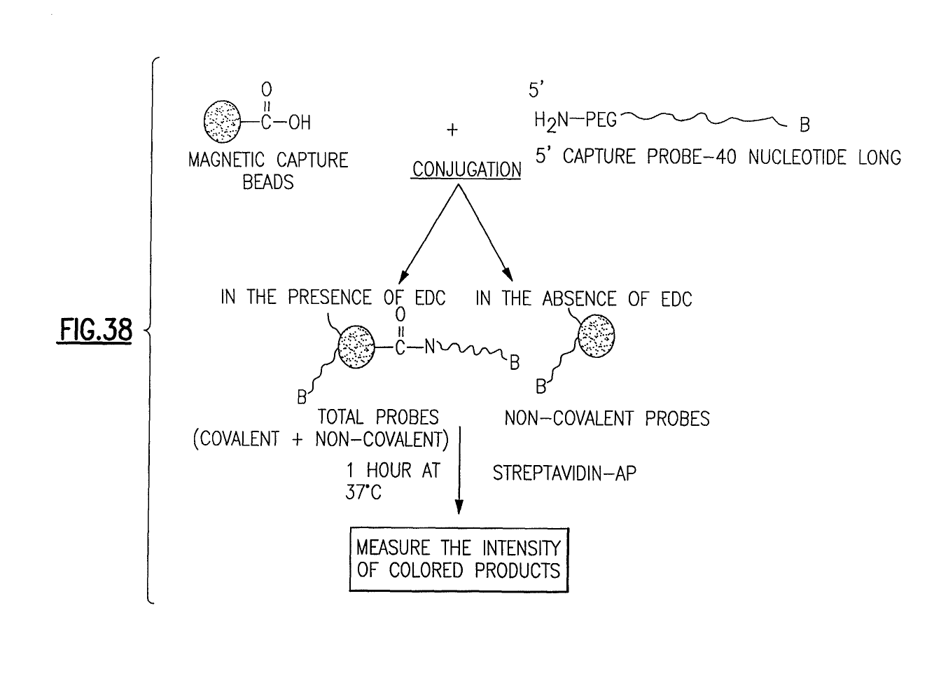

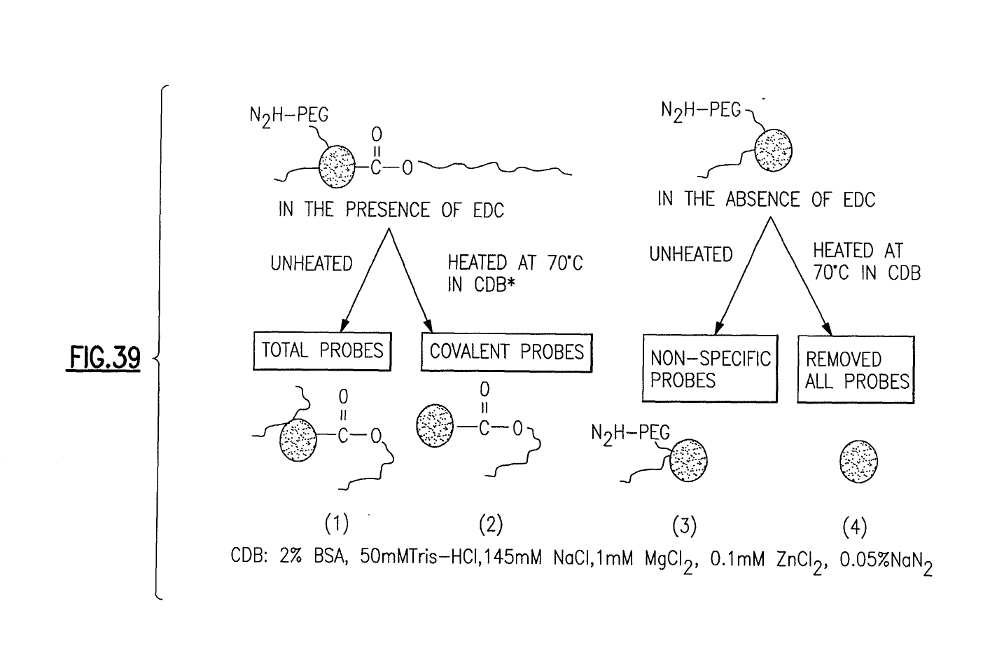

As illustrated in FIG. 38, a systematic method to evaluate the use of a solid phase for probe conjugation is presented. The methodology identifies covalent linkages that improve specificity of a dual bead assay. This approach can be used to evaluate treatment of solid phase (i.e., coating of a solid surface such as the surface of a bead or a surface on a biodisc) to see whether the treatment improves the solid phase conjugation efficiency. As a first step, probes are tagged with an appropriate molecule for detection and measurement of the amount of probe bound at a later time. By way of non-limiting example, a biotin moiety (B) can be attached at the 3′ end of a DNA probe. Next, the probe is conjugated in the presence or absence of a cross-linking agent, e.g., EDC (1-Ethyl 3-3 dimethylaminopropyl carbodiimide-HCl). In the presence of a cross-linking agent, a probe will be conjugated both covalently and non-covalently. Alternatively, in the absence of the cross-linking agent, a probe will only be absorbed to the bead non-covalently. After the appropriate washing steps are performed, a detection agent is added that binds specifically to the biotin molecule previously tagged to the probe. For example, streptavidin-alkaline phosphatase (S-AP) is added to the probe-bound beads, and the S-AP binds specifically to the biotinylated probes. Next, alkaline phosphatase substrate is added to the sample. This substrate develops color upon loss of a phosphate group, and the intensity of the color correlates with the amount of probes bound to the beads. After an appropriate incubation period, the solution is isolated and the optical density of the solution at an appropriate wavelength is determined with a spectrophotometer or microtiter plate reader.

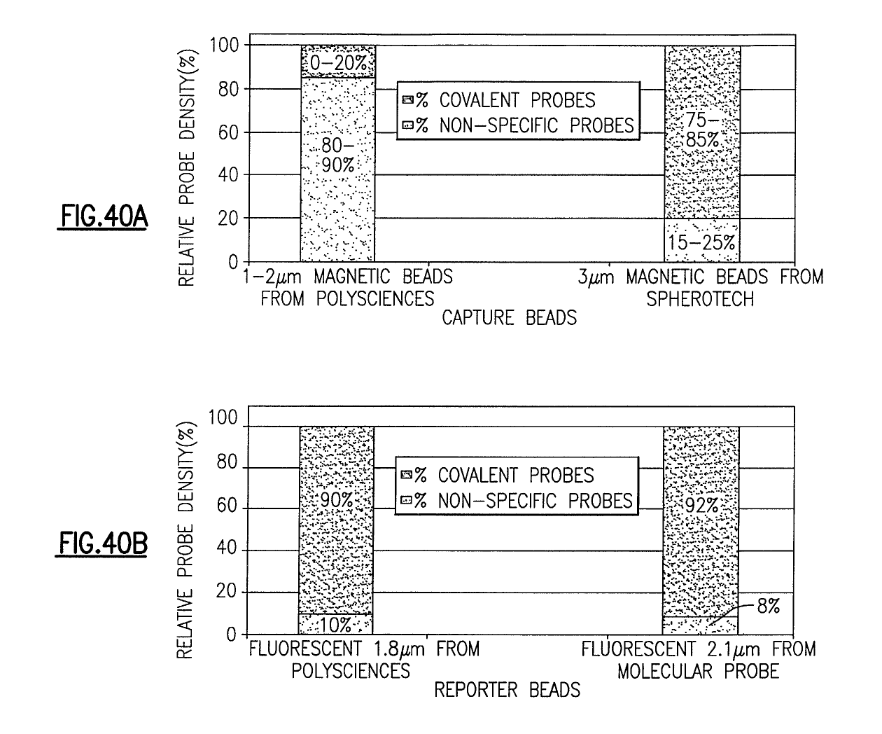

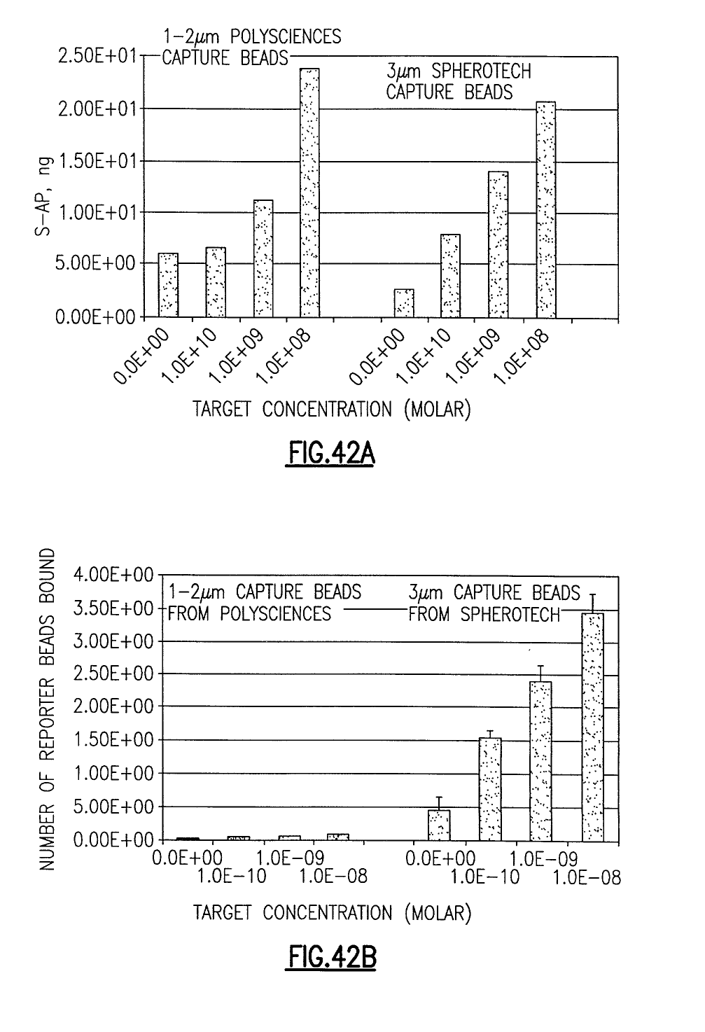

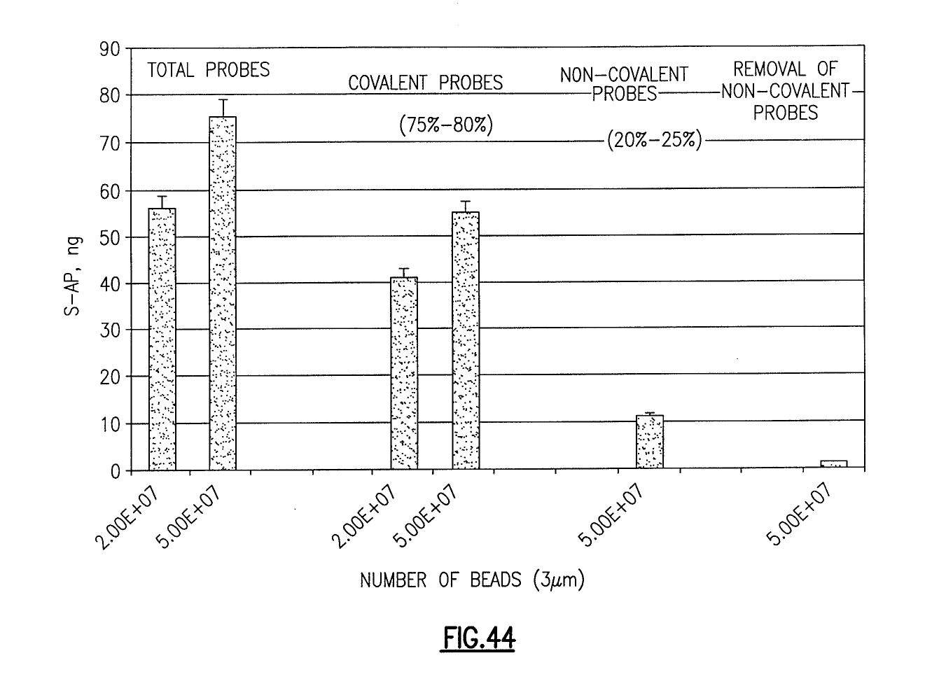

Referring to FIG. 39, there is illustrated conjugation of an oligonucleotide probe onto a carboxylated bead. Conjugation of probes may be carried out covalently or non-covalently. In a dual bead assay, covalent probe conjugation is preferred over non-covalent conjugation as discussed in further detail in connection with FIGS. 42A and 42B. This conjugation process is performed prior to Step I of the dual bead assay as presented in FIGS. 11A, 11B, 12A, and 12B. The amount of probe covalently bound to the solid surface may be evaluated by determining the amount of probe that binds to the solid phase covalently and non-covalently, i.e., non-specifically, in the presence and absence of a crosslinking agent (e.g., EDC). The percentage of non-covalently bound probe can be determined according to the formula 100% * N/T, and the percentage of covalently bound probe can be determined by the formula 100% * (T-N)/T, wherein “T” represents the total amount of signal obtained in the presence of a cross-linking agent (i.e., the total amount of covalently and noncovalently bound probe) and “N” represents the total amount of signal obtained when no crosslinking agent is used. Alternatively, the amount of probes conjugated covalently can be obtained directly if all non-covalently bound probes are removed prior to the addition of the S-AP. This can be conveniently achieved by heating the beads to 70° C. prior to the step of adding the S-AP. If the percentage of non-covalently bound probe is less than 20%, the beads being tested can be used as solid phase for covalent conjugation. Results of an application of this methodology are presented in FIGS. 40A, 40B, and 44 (see Example 3 for details).

As depicted in FIGS. 40A and 40B, the 1.8 μm, 2.1 μm, and 3 μm beads provide suitable solid phase for covalent probe conjugation with at least 75% conjugation efficiency. The 2.1 μm beads, however, may not be suitable for covalent conjugation of probes due to their low covalent conjugation efficiency of less than 21%.

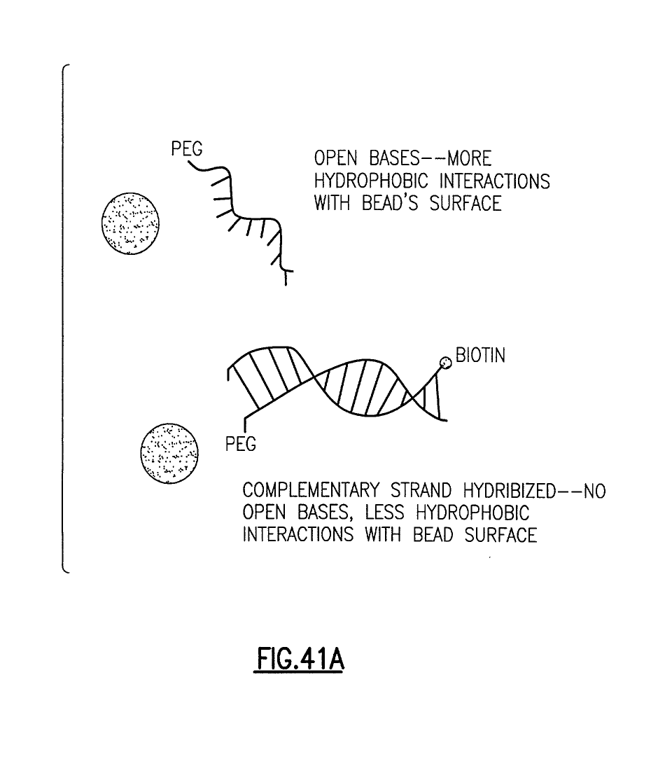

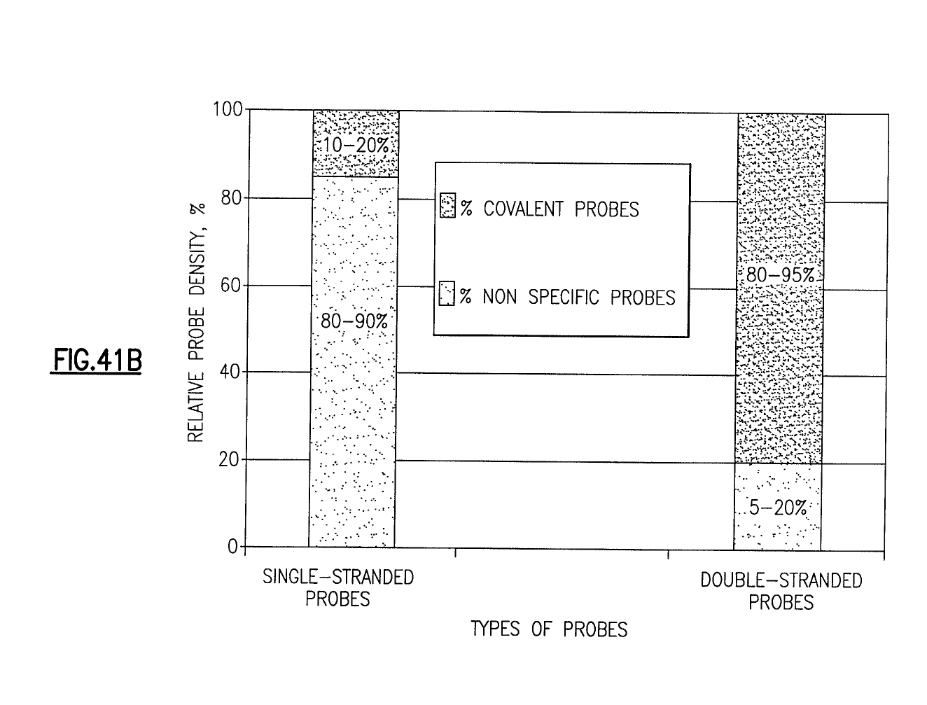

Various embodiments of the invention utilize nucleic acid molecules as probes. FIG. 41A shows the structural differences between single stranded and double stranded DNA in order to illustrate how the single stranded DNA can more readily bind non-covalently to a solid phase. Single-stranded DNA has hydrophobic base side chains that can readily absorb to a solid phase non-covalently. In contrast, with double-stranded DNA hydrophobic base interaction with a solid phase does not generally occur and non-covalent or non-specific binding is limited in comparison to a single-stranded DNA molecule. Thus, in various embodiments of the invention, double stranded DNA can be utilized in place of single-stranded DNA, thereby enhancing DNA binding to a solid phase by covalent linkage (FIG. 41B). After covalent binding of one of the strands of the double-stranded DNA probe to the solid phase, the non-covalently bound strand may be removed by heating the sample to 70° C. in the appropriate buffer. Under these conditions, the double stranded DNA are separated, and only single strand DNA probe that is covalently attached to the bead ramain and is used to capture the target. Experimental details regarding the use of double stranded DNA for covalent probe conjugation is described in further detail below in Example 4.

In various embodiments of the invention, heat treatment can be used to selectively remove non-covalently bound probe(s) from a solid phase. This method is useful when, for example, despite all optimizations with respect to the type of the solid phase, treatment of the solid phase, and the use of double stranded DNA, non-covalent binding to the solid phase is still problematic. The conditions for the heat treatment have been optimized; the optimal buffer consists of: 2% BSA, 50 mM Tris-HCl, 145 mM NaCl, 1 mM MgCl2, 0.1 mM ZnCl2. The treatment is done at a temperature less than or equal to approximately 70° C., since at higher temperatures, the magnetic beads can lose their magnetic properties.

In other embodiments of the invention, the methodology presented herein to determine optimal conditions to obtain covalent linkages that improve specificity of a dual bead assay can be applied to a disc surface that is used as a solid phase. Similarly, the invention provides in other embodiments analogous to those described herein above to evaluate solid surfaces for protein binding. For example, such an application would be useful where the probe utilized is an antigen or antibody.

Referring now to FIG. 42A, there is shown a bar graph of results collected from an enzyme assay detecting targets bound to probes on two different capture beads for use in a dual bead assay. As illustrated above in FIG. 40A, the 1-2 μm beads have a covalent binding efficiency up to 20% and the rest of the probes bind non-covalently and the covalent binding efficiency of the 3 μm beads is between 75-85%. The data shown in FIG. 42A indicates that both of the tested beads bind a similar amount of target regardless of whether the probe is bound covalently or noncovalently. This suggests that covalent binding is not necessary in an enzyme assay format.

In contrast to FIG. 42A, FIG. 42B represents results of a dual bead assay designed to examine the number of reporter beads captured by the same capture beads used in FIG. 42A. The results shown in FIG. 42B indicate that covalent binding of the probe to the capture bead is necessary to enhance the sensitivity of the assay. In this particular embodiment of the present invention, the 3 μm capture bead contains more covalently bound probes than the 1-2 μm beads, as mentioned above. This allows the retention of the reporter bead in the dual bead complex since covalently tethered probes on the capture bead have higher bond strength than non-covalently bound probes.

As mentioned in the summary of the invention above, the surface of the beads or solid phase may be uneven which limits the probe accessibility to the target in solution. Probe linkers may be used to extend the length of the probes to increase probe target accessibility as discussed with reference to FIG. 41A.

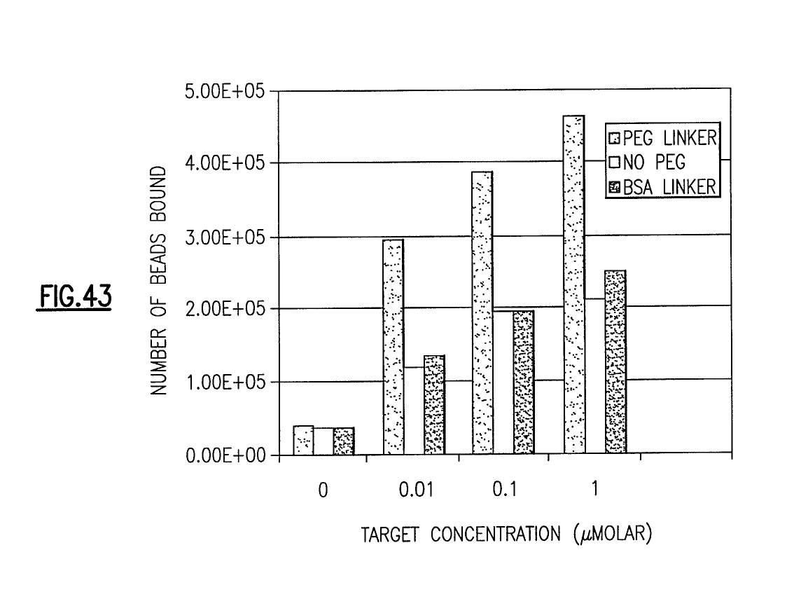

With reference now to FIG. 43, there is presented data collected from a dual bead assay showing enhanced target binding using PEG as a linker. Linkers may increase the assay sensitivity by approximately 50% or more. The use of linkers also decreases non-specific reporter bead binding to the capture beads. In this embodiment of the present invention, probes are attached to a solid phase by way of a linker molecule. The use of a linker molecule makes the probe longer and more rigid. These two properties increase the accessibility of the probe(s), and, therefore, maximize the efficiency of target capture and the sensitivity of the dual bead assay. As known to those skilled in the art, various linker molecules can be used that satisfy the criteria described herein. By way of non-limiting example, bovine serum albumin (BSA) or polyethylene glycol (PEG) can be used as linker molecules. In certain embodiments of the invention, the linker can be a series of 3 to 10 PEG molecules that are attached covalently to the 5′ end of a DNA probe. Details relating to the use of PEG as a linker molecule are described below in Example 5.

With reference now to FIG. 44, there is shown a bar graph demonstrating determination of percent covalent probe density on 3 μm Spherotech beads. These graphs represent signals generated from an enzyme assay using biotinylated probes and streptavidin-linked alkaline phosphatase enzyme reactions. As discussed with reference to FIG. 39, the covalent conjugation efficiency can be calculated by determining the total amount of probes bound to non heat-treated beads. A separate aliquot of the beads is then heated to remove the non-covalently bound probes and the amount of covalent probes is then determined using the enzyme assay as described in Example 3 below. With these data, the percentage of covalent probe binding can then be determined using the following formula: H/T*100 where H represents signal from heat treated beads and T is the total signal from the non-heat treated beads.

FIG. 45 is a bar graph presentation demonstrating the pretreatment of the beads with various blocking agents including detergents. Decreasing non-specific bead binding is critical in the dual bead assay since the assay sensitivity is inversely related to the baseline signal which is the non-specific binding of the reporter beads to the capture beads. Thus the lower the baseline, the more sensitive the assay becomes. As illustrated, the use of salmon sperm DNA worked best in reducing the nonspecific binding relative to the other blocking agents tested in this experiment. Salmon sperm DNA blocking reduced non-specific binding by approximately 10 fold. Salmon sperm DNA is, therefore, a preferred method for blocking non-specific bead binding in one aspect of the present invention. Other blocking agents may also be used including BSA, Denhardt's solution, and sucrose. Preferably, beads should be blocked by an appropriate blocking agent after conjugation and heat treatment as shown in FIG. 39 or prior to Step I in FIGS. 11A, 11B, 12A, and 12B above to increase the dual bead assay sensitivity.

Experimental Details

While this invention has been described in detail with reference to the drawing figures, certain examples and further illustrations of the invention are presented below.

### EXAMPLE 1

The two-step hybridization method demonstrated in FIG. 12A was used in performing the dual bead assay of this example.

### A. Dual Bead Assay

In this example, the dual assay in carried out to detect the gene sequence DYS that is present in male but not in female. The assay is comprised of 3μ magnetic and capture beads coated with covalently attached capture probe; 2.1μ fluorescent reporter beads coated with a covalently attached sequence specific for the DYS gene, and target DNA molecule containing DYS sequences. The target DNA is a synthetic 80 oligonucleotide sequence. The capture probe and reporter probes are 40 nucleotides in length and are complementary to DYS sequence but not to each other.

The specific methodology employed to prepare the assay involved treating 1×10⁷ capture beads and 2×10⁷ reporter beads in 100 microgram per milliliter Salmon Sperm DNA for 1 hr. at room temperature. This pretreatment will reduce non-covalent binding between the capture and reporter beads in the absence of target DNA as shown in FIG. 45. The capture beads were concentrated magnetically with the supernatant being removed. A 100 microliter volume of the hybridization buffer (0.2 M NaCl, 1 mM EDTA, 10 mM MgCl₂, 50 mM Tris HCl, pH 7.5, and 5×Denhart's mixture, 10 microgram per milliliter denatured salmon sperm DNA) were added to the capture beads and the beads were re-suspended. Various concentrations of target DNA ranging from 1, 10, 100, 1000 femto were added while mixing at 37° C. for 2 hours. The beads were magnetically concentrated and the supernatant containing target DNA was removed. A 100 microliter volume of wash buffer (145 mM NaCl, 50 mM Tris pH 7.5, 0.1% SDS, 0.05% Tween, 0.25% NFDM, 10 mM EDTA) was added and the beads were re-suspended. The beads were magnetically concentrated and the supernatant was again removed. The wash procedure was repeated twice.

A 2×10⁷ amount of reporter beads in 100 microliter hybridization buffer (0.2 M NaCl, 1 mM EDTA, 10 mM MgCl₂, 50 mM Tris HCl, pH 7.5, and 5×Denhart's mixture, 10 microgram per milliliter denatured salmon sperm DNA) were then added to washed capture beads. The beads were re-suspended and incubated while mixing at 37° C. for an additional 2 hours. After incubation the capture beads were concentrated magnetically, and the supernatant containing unbound reporter beads were removed. A 100 microliter volume of wash buffer (145 mM NaCl, 50 mM Tris pH 7.5, 0.1% SDS, 0.05% Tween, 0.25% NFDM, 10 mM EDTA) was added and the beads were re-suspended. The beads were magnetically concentrated and the supernatant was again removed. The wash procedure was repeated twice.

After the final wash, the beads were re-suspended in 20 microliters of binding buffer (50 mM Tris, 200 mM NaCl, 10 mM MgCl₂, 0.05% Tween 20, 1% BSA). A 10 microliter volume was loaded on to the disc that was prepared as described below in Part B of this example.

### B. Preparation of the Disc

A gold disc was coated with maleic anhydride polystyrene. An amine DNA sequence complementary to the reporter probes (or capture agent) was immobilized on to the discrete reaction zones on the disc. Prior to sample injection, the channels were blocked with a blocking buffer (50 mM Tris, 200 mM NaCl, 10 mM MgCl₂, 0.05% Tween 20, 1% BSA, 1% sucrose) to prevent non-covalent binding of the dual bead complex to the disc surface. A perspective view of the disc assembly showing capture agents 220, the capture zones 170, and fluidic circuits as employed in the present invention is illustrated in detail in FIGS. 25A-25D. Alternatively, if the reporter beads are coated with Streptavidin, a capture zone could be created with the capture agent such as BSA Biotin which could be immobilized on to the disc (pretreated with Polystyrene) by passive absorption. A perspective view of the disc assembly showing the use of biotin capture agents is presented in FIGS. 26A-26D. Various methods for use in this type of anchoring of beads onto the disc are also shown in FIGS. 15A-15B, 17, 19A-19C, and 23A-23B.

### C. Capture of Dual Bead Complex Structure on the Disc

A 10 microliter volume of the dual bead mixture prepared as described in Part A above was loaded in to the disc chamber and the injection ports were sealed. To facilitate hybridization between the reporter probes on the reporter beads and the capture agents, the disc was centrifuged at low speed (less than 800 rpm) upto 15 minutes. The disc was read in the CD reader at the speed 4× (approx. 1600 rpm) for 5 minutes. Under these conditions, the unbound magnetic capture beads were centrifuged away from the capture zone. The magnetic capture beads that were in the dual bead complex remained bound to the reporter beads in the capture zone. The steps involved in using the disc to capture and analyze dual bead complexes are presented in detail in FIGS. 25A-25D, 26A-26D, and 27A-27D.

### D. Quantification of the Dual Bead Complex Structures

The amount of target DNA captured could be enumerated by quantifying the number of capture magnetic beads and the number of reporter beads since each type of bead has a distinct signature.

### EXAMPLE 2

### A. Dual Bead Assay Multiplexing

In this example, the dual bead assay is carried out to detect two DNA targets simultaneously. The assay is comprised of 3μ magnetic capture bead. One population of the magnetic capture bead is coated with capture probes 1 which are complementary to the DNA target 1, another population of magnetic capture beads is coated with capture probes 2 which are complementary to the DNA target 2. Alternatively two different types of magnetic capture beads may be used. There are two distinct types of reporter beads in the assay. The two types may differ by chemical composition (for example Silica and Polystyrene) and/or by size. Various combinations of beads that may be used in a multiplex dual bead assay format are depicted in FIG. 32. One type of reporter bead is coated with reporter probes 1, which are complementary to the DNA target 1. The other reporter beads are coated with reporter probes 2, which are complementary to the DNA target 2. Again the capture probes and the reporter probes are complementary to the respective targets but not to each other.

The specific methodology employed to prepare the dual bead assay multiplexing involved treating 1×10⁷ capture beads and 2×10⁷ reporter beads in 100 μg/ml salmon sperm DNA for 1 hour at room temperature. This pretreatment will reduce non-covalent binding between the capture and reporter beads in the absence of target DNA. The capture beads were concentrated magnetically with the supernatant being removed. A 100 microliter volume of the hybridization buffer (0.2 M NaCl, 1 mM EDTA, 10 mM MgCl₂, 50 mM Tris HCl, pH 7.5, and 5×Denhart's mixture, 10 microgram per milliliter denatured salmon sperm DNA) were added and the beads were re-suspended. Various concentrations of target DNA ranging from 1, 10, 100, 1000 femto moles were added to the capture beads suspension. The suspension was incubated while mixing at 37° C. for 2 hours. The beads were magnetically concentrated and the supernatant containing target DNA was removed. A 100 microliter volume of wash buffer (145 mM NaCl, 50 mM Tris pH 7.5, 0.1% SDS, 0.05% Tween, 0.25% NFDM, 10 mM EDTA) was added and the beads were re-suspended. The beads were magnetically concentrated and the supernatant was again removed. The wash procedure was repeated twice.

A 2×10⁷ amount of reporter beads in 100 microliter hybridization buffer (0.2 M NaCl, 1 mM EDTA, 10 mM MgCl₂, 50 mM Tris HCl, pH 7.5, and 5×Denhart's mixture, 10 microgram per milliliter denatured salmon sperm DNA) were then added to washed capture beads. The beads were re-suspended and incubated while mixing at 37° C. for an additional 2 hours. After incubation the capture beads were concentrated magnetically, and the supernatant containing unbound reporter beads were removed. A 100 microliter volume of wash buffer (145 mM NaCl, 50 mM Tris pH 7.5, 0.1% SDS, 0.05% Tween, 0.25% NFDM, 10 mM EDTA) was added and the beads were re-suspended. The beads were magnetically concentrated and the supernatant was again removed. The wash procedure was repeated twice.

After the final wash, the beads were re-suspended in 20 microliters of binding buffer (50 mM Tris, 200 mM NaCl, 10 mM MgCl₂, 0.05% Tween 20, 1% BSA). A 10 microliter volume of this solution was loaded on to the disc that was prepared as described in below in Part B of this example.

### B. Disc Preparation

A gold disc was coated with maleic anhydride polystrene as described. Distinct reaction zones were created for two types of reporter beads. Each reaction zone consisted of amine DNA sequences complementary to the respective reporter probes (or capture agents). Prior to sample injection, the channel were blocked with a blocking buffer (50 mM Tris, 200 mM NaCl, 10 mM MgCl₂, 0.05% Tween 20, 1% BSA, 1% sucrose) to prevent non-covalent binding of the dual bead complex to the disc surface. Alternatively, magnetic beads employed in a multiplexing dual bead assay format may be detected using a magneto-optical disc and drive. The chemical reaction zones, in the magnetic disc format, are replaced by distinctly spaced magnetic capture zones as discussed in conjunction with FIG. 37.

### C. Capture of Dual Bead Complex Structure on the Disc

A 10 microlitre volume of the dual bead mixture prepared as described above in Part A of this example, was loaded in to the disc chamber and the injection ports were sealed. To facilitate hybridization between the reporter probes on the reporter beads and the capture agents, the disc was centrifuged at low speed (less than 800 rpm) for up to 15 minutes. The disc was read in the CD reader at the speed 4× (approx. 1600 rpm) for 5 minutes. Under these conditions, the unbound magnetic capture beads were centrifuged to the bottom of the channels. The reporter beads bound to the capture zone via hybridization between the reporter probes and their complementary agent.

### D. Quantification of the Dual Bead Complex Structures

The amount of target DNA 1 and 2 captured could be enumerated by quantifying the number of the respective reporter beads in the respective reaction zones.

### EXAMPLE 3

This experiment was performed to determine the amount of covalently conjugated probe on different beads to determine which bead type is best for covalent probe linking.

### A. Conjugation

Magnetic beads (1-2 μm) from Polysciences, magnetic beads (3 μm) from Spherotech, fluorescent beads (1.8 μm) from Polysciences and fluorescent beads (2.1 μm) from Molecular Probes were evaluated in this example. Approximately 5×10⁸ beads were used per conjugation reaction. The beads were washed and resuspended in 0.05 M MES buffer (2-N-morpholeno-ethanesulphonic acid), pH 6.0 and activated for 15 minutes by the addition of 0.1M EDC (1-ethyl 3-3 dimethylaminopropyl carbodimide-HCl). After activation, the pH of the bead solution was adjusted to ˜7.5 with NaOH. Then 0.5 nanomoles of biotinylated probes were added to the solution. The probes were allowed to conjugate for 2-3 hours at room temperature on a rotating mixer. The beads were then magnetically concentrated and the supernatant was collected. To estimate the amount of biotinylated probes bound to the beads, the optical density (at 260 nm) of the supernatant was measured before and after the conjugation.

### B. Determination of Covalent Conjugation Efficiency

Typically 1 to 5×10⁷ beads, conjugated with biotinylated probes as discussed above, were used in the determination of covalent conjugation efficiency of the probes. These beads were washed three times in wash buffer and were resuspended in 200 μl CDB (145 mM NaCl, 50 mM Tris HCl, 2% BSA, 1 mg/ml MgCl₂, 0.1 mM ZnCl₂, 0.05% NaN₂). The beads were then magnetically concentrated, and the supernatant was removed. The beads were resuspended in 100 μl CDB containing 550 ng/ml streptavidin-alkaline phosphatase (S-AP) and incubated for 1 hour at 37° C. to allow sufficient time for the streptavidin to bind to the biotin on the probe. Following incubation with S-AP, the beads were magnetically concentrated, and the supernatant containing unbound S-AP was removed. The beads were washed three times in wash buffer. Next 100 μl of p-nitrophenylphosphate (pNPP), a substrate for alkaline phosphatase at a concentration of 3.7 mg/ml in 0.1 M Tris-HCl, pH 10 was added to the beads at fixed time intervals to minimize the variation due to difference in incubation time. The incubation time with the substrate was varied (from 2 min upto 30 min) as needed to obtain reliable OD at 405 nm since time for color development varies depending upon the concentration of probe. The optical density obtained from a spectrophotometer at 405 nm wavelength was proportional to the amount of probes bound to the beads.

The results of the experiment are presented in FIGS. 44A and 44B. As indicated, 87% of the probes that bound to the 1-2 μm magnetic beads from Polysciences were non covalently bound, as compared to 15% of non-covalently bound probes on the 3 μm Spherotech beads.

Referring to FIGS. 42A and 42B, data showing a correlation between the covalent conjugation efficiency and the sensitivity of the dual bead assay is presented. These results indicate that with higher covalent conjugation efficiency, the more sensitive and specific the dual bead assay is. The amount of covalenty bound probes may be calculated by repeating steps in this Part B after performing the steps in Part C below. The calculation of the amount of covalent binding is presented in FIG. 44.

### C. Heat Treatment in the Removal of Non-Covalently Bound Probes

After determining which bead type has the desired covalent conjugation efficiency, the steps in Parts A and B above may be repeated using non-biotinylated probes and the appropriate bead type for use in a dual bead assay.

Following conjugation, the non-covalently bound probes could be selectively removed by heat treatment of the beads. For this purpose, up to 3×10⁷ beads were resuspended in 100 μl of CDB solution heated at 70° C. for 10 minutes. The beads were then immediately magnetically concentrated and the supernatant was removed. The beads were washed twice in wash buffer and once in CDB and resuspended in 100 μl CDB. At this point the beads are now ready for use in a dual beads test.

### EXAMPLE 4

Experiments were also done to evaluate the use of double stranded DNA during probe conjugation to increase the covalent conjugation efficiency of the DNA probe on the solid phase.

### A. Formation of Double Stranded DNA

The capture probe utilized was 40 nucleotides in length and contained an aminogroup (NH₂) at the 5′ end and several chains of PEG (polyethylene glycol) linker. The strand complimentary to the aminated probe used in this experiment was 40 nucleotides in length and contained a biotin group at the 5′ end. A hybridization reaction was carried out with an excess of complementary probes under stringent conditions at 37° C.

### B. Conjugation of the Double-Stranded DNA Probe onto Beads

Magnetic beads (1-2 μm) from Polysciences, magnetic beads (3 μm) from Spherotech, fluorescent beads (1.8 μm) from Polysciences and fluorescent beads (2.1 μm) from Molecular Probes were evaluated in this example. Approximately 5×10⁸ were used per conjugation reaction. The beads were washed and resuspended in 1 ml of 0.05 M MES buffer (2-N-morpholeno-ethanesulphonic acid), pH 6.0 and activated for 15 minutes by the addition of 0.1M EDC (1-ethyl 3-3 dimethylaminopropyl carbodimide —HCl). After activation, the pH was adjusted to ˜7.5 with NaOH. A volume of 0.5 nanomoles of probes were then added to the solution. The probe conjugation was carried out for 2-3 hours at room temperature on a rotating mixer. The beads were then magnetically concentrated and the supernatant was removed. To estimate the amount of probes bound to the beads, the optical density at 260 nm of the supernatant was measured before and after the conjugation.

After the conjugation, all unreacted carboxyl groups on the beads were blocked with 1 ml 0.1 M Tris-HCl pH 7.5 for 1 hour at room temperature on a mixer. The beads were then blocked for 30 minutes in 1 ml of 10 mg/ml BSA in PBS at room temperature on the mixer to block any unspecific protein binding sites. After blocking, the beads were washed three times with PBS and resuspended in storage buffer (PBS with 10 mg/ml BSA, 5% glycerol, 0.1% sodium azide).

### C. Determination of Covalent Conjugation Efficiency

An aliquot of 2×10⁸ magnetic beads was taken out from the above conjugated beads and pre-treated with 0.1 mg/ml salmon sperm DNA for 1 hour at room temperature. The beads were then washed 3 times in wash buffer and resuspended in 200 μl CDB. Then 200 picomoles of blocking probes and 100 μl of hybridization buffer were added to the bead solution. The blocking probes were allowed to hybridize for two hours at 37° C. After hybridization, the beads were magnetically concentrated and the supernatant was removed. The beads were then washed three times in wash buffer using by magnetic concentration. The beads were resuspended with 100 μl of buffer containing 550 ng/ml streptavidin-alkaline phosphatase (S-AP) and incubated for 1 hour at 37° C. Following incubation with SAP, the beads were magnetically concentrated, and the supernatant containing unbound S-AP was removed. The beads were washed three times in wash buffer. Next 100 μl of p-nitrophenylphosphate (pNPP), a substrate for alkaline phosphatase at a concentration of 3.7 mg/ml, was added to the beads at fixed time intervals to minimize the variation due to difference in incubation time. The time for color development varies depending upon the concentration of probe. The incubation time with the substrate was varied from 2 min up to 30 min as needed to obtain reliable OD at 405 nm. The optical density at 405 nm was proportional to the amount of probes bound to the beads. The results from one of these double stranded conjugation experiments are presented in FIGS. 41A and 41B above.

### D. Use of Heat Treatment to Separate Complimentary Strands from Capture Probes

An aliquot of 100 μl of beads were heated for 10 min. at 70° C. Magnetically concentrate the beads and take out the supernatant promptly. Wash once in hot wash buffer and once in CDB. Then resuspend in CDB.

### EXAMPLE 5

Experiments were also conducted to test the use of linkers of longer spacers to increase the efficiency of conjugation and the accessibility and rigidity of the probes attached to a solid phase. In these experiments, the capture and reporter probes were 40 nucleotides in length. These synthetic nucleotide sequences were specific to the analyte of interest. In this example, the 5′ end of the capture probe and 3′ end of the reporter probe contained conjugated 3 polyethylene glycol moieties. These covalently bound linkers were introduced to the probes during probe synthesis. Data collected from one of these experiments are depicted in FIG. 43 above. As shown in FIG. 43, the use of linkers significantly increases the sensitivity of the dual bead assay.

The beads used in this particular assay were 3 μm magnetic beads from Spherotech and 2.1 μm reporter beads from Molecular Probes. The probes were covalently conjugated to the beads as described above. An aliquot of 2×10⁷ of probe conjugated capture beads and 6×10⁷ of reporter beads per assay were washed three times with PBS. After washing, the beads were pretreated with 100 μg/ml of salmon sperm DNA in water for one hour at room temperature. The beads were washed three times in wash buffer (0.145M NaCl, 50 mM Tris-HCl pH 7.5, 0.5% Tween-20), once with hybridization buffer (50 mM Tris-HCl pH 7.5, 0.1M NaCl, 10 mM MgCl, 1 mM EDTA pH 7.5) and re-suspended in hybridization buffer containing 100 μg/ml DNA, and 5×Denhart's mixture.

The two-step hybridization method, as presented in FIG. 12A, was employed in performing the dual bead assay of this example. Different concentrations of a single target were used including Control (0 femtomole), 10 femtomole, 1 femtomole, 0.1 femtomole, 0.01 femtomole, 0.001 femtomole, 0.0001 femtomole diluted in hybridization buffer containing 100 μg/ml of salmon sperm DNA and 5×Denhart's solution. The various target solutions were then mixed with the capture beads and incubated at 37° C. for 2 hours to allow ample time for target hybridization to the capture probe on the beads. After hybridization the hybridized capture beads were washed three times with wash buffer, once with hybridization buffer, and re-suspended in 100 μl hybridization buffer including 100 μg/ml DNA, and 5×Denhart's mixture. The capture bead solution, containing hybridized target, was then mixed with 100 μl of reporter beads and incubated at 37° C. for 2 hours while continuously mixing. Then washed 6 times with new wash buffer (145 mM NaCl, 50 mM Tris-HCl pH 7.5, 05% Tween 20, 0.1% SDS, 0.25% NFDM) and once with PBS. The washed solution containing the dual bead complexes was then re-suspended with 250 μl PBS. The fluorescent signal from the reporter beads were then quantified using a fluorimeter.

Results showed that when 3 PEG linkers were introduced into the capture probe, it lowered the background in dual bead assays and improved the assay sensitivity significantly as compared to probes without linkers.

### CONCLUDING STATEMENT

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

What is claimed is:

1. A method of evaluating a solid phase for use in a dual bead assay, the method comprising the steps of:
selecting a test solid phase;
binding a probe to the test solid phase in the presence or absence of a cross-linking agent;
determining a total amount of probe bound to the test solid phase in the presence or absence of a cross-linking agent;
determining a percentage of probe bound covalently to the solid phase;
determining an amount of probe bound to the solid phase non-covalently; and
calculating a percentage of probe bound covalently to the solid phase, wherein if no less than a pre-determined minimum threshold of the probes is bound covalently, the solid phase is suitable for use in a dual bead assay.

2. The method according to claim 1 wherein said solid phase is a bead.

3. The method according to claim 2 wherein said bead is a magnetic bead.

4. The method according to claim 1 wherein said solid phase is a surface on a bio-disc.

5. The method according to claim I wherein said probe is nucleic acid.

6. The method according to claim 5 wherein said nucleic acid is double stranded.

7. The method according to claim 1 wherein said probe is a protein.

8. The method according to claim any of the claims 5, 6, or 7 wherein said probe further comprises a linker.

9. The method according to claim 8 wherein said linker is at least 1 polyethylene glycol moiety.

10. The method according to claim 8 wherein said linker is a polymer consisting of polyethylene glycol.

11. The method according to any of the claims 5, 6, 7, 9, or 10 wherein said probe is biotinylated.

12. The method according to claim 11 wherein said probe is quantified by an enzyme assay.

13. The method according to claim 1 wherein said test solid phase is attached to a bio-disc.

14. The method according to claim 1 wherein the said minimum threshold for covalent probe binding is 50%.

15. The method according to claim 1 wherein the said minimum threshold for covalent probe binding is 80%.

16. A method for DNA conjugation onto a solid phase for determining the suitability of a test solid phase for use in a dual bead assay, said method comprising the steps of:
selecting a test solid phase;
conjugating a probe onto the test solid phase;
washing the solid phase employing a conjugate dilution buffer;
heat treating the solid to thereby remove the non-covalently bound probes, and;
calculating the percentage of probes bound covalently to the solid phase.

17. The method according to claim 16 wherein if no less than a predetermined minimum threshold of probe is bound covalently, the solid phase is suitable for use in a dual bead assay.

18. The method according to claim 17 wherein the said minimum threshold for covalent probe binding is 50%.

19. The method according to claim 17 wherein the said minimum threshold for covalent probe binding is 80%.

20. The method according to either claim 18 or 19 wherein said solid phase is a bead.

21. The method according to claim 20 wherein said bead is a magnetic bead.

22. The method according to claim 21 wherein said bead is a 3 μm bead.

23. The method according to claim 22 wherein said conjugation is performed in the presence of a cross linking agent.

24. The method according to claim 23 wherein said cross-linking agent is EDC.

25. The method according to claim 16 wherein said probe is single stranded.

26. The method according to claim 16 wherein said probe is double stranded.

27. The method according to claim 16 wherein said conjugation is partially covalent.

28. The method according to claim 16 wherein said conjugation is completely covalent.

29. An optical bio-disc, comprising:
a substrate having a tracking groove formed therein;
a reflective layer formed on at least a portion of said substrate so that an incident beam of electromagnetic energy may track along said groove;
an active layer associated with said substrate; and
a capture agent having an affinity for said active layer so that said capture agent is immobilized by said active layer so that a percentage of said capture agent bound covalently to said active layer may be calculated.

30. The optical bio-disc according to claim 29 wherein said capture agent is a strand of DNA.

31. The optical bio-disc according to claim 30 wherein said strand of DNA is a single strand of DNA.

32. The optical bio-disc according to claim 30 wherein said strand of DNA includes a double strand of DNA.

33. The optical bio-disc according to claim 29 wherein the capture agent is an antibody.

34. The optical bio-disc according to claim 29 wherein the capture agent is an antigen.

35. The optical bio-disc according to claim 29 wherein the capture agent is biotin.

36. The optical bio-disc according to claim 29 wherein the capture agent is streptavidin.

37. The optical bio-disc according to any one of claims 30, 31, 32, 33, 34, 35, or 36 wherein said active layer is formed from a polystyrene co-maleic anhydride.

38. The optical bio-disc according to claim 37 wherein said capture agent contains an active group that binds covalently to said active layer.

39. The optical bio-disc according to claim 38 wherein said active group is an amino group.

40. The optical bio-disc according to claim 38 wherein said capture agent binds to an anchor agent to thereby locate said anchor agent within said target zone.

41. The optical bio-disc according to claim 40 wherein said anchor agent is a DNA strand.

42. The optical bio-disc according to claim 40 wherein said anchor agent is an antibody.

43. The optical bio-disc according to claim 40 wherein said anchor agent is an antigen.

44. The optical bio-disc according to claim 40 wherein said anchor agent is biotin.

45. The optical bio-disc according to claim 40 wherein said anchor agent is streptavidin.

46. The optical bio-disc according to claim 40 wherein said anchor agent is attached to a bead to thereby locate said bead within the target zone.

47. The optical bio-disc according to claim 46 wherein said bead is a capture bead.

48. The optical bio-disc according to claim 46 wherein said bead is a reporter bead.

49. The optical bio-disc according to claim 47 wherein said capture bead and reporter bead are linked by a target molecule thereby forming a dual bead complex which is tethered to said capture agent within said target zone.

50. The optical bio-disc according to claim 49 wherein an incident beam of electromagnetic radiation inspects said dual bead complex.

51. An optical bio-disc, comprising:
a substrate having encoded information associated therewith, said encoded information being readable by a disc drive assembly to control rotation of the disc;
a target zone associated with said substrate, said target zone disposed at a predetermined location relative to said substrate;
an active layer associated with said target zone; and
a plurality of capture agents attached to said active layer so that when said substrate is rotated, said capture agents remain attached to said active layer to thereby maintain a number of said capture agents within said target zone so that when a dual bead complex including covalently bound probes is introduced into said target zone, said capture agent sequesters said dual bead complex therein to thereby allow detection of captured dual bead complex.

52. The optical bio-disc according to claim 51 wherein said capture agent is a single stranded oligonucleotide sequence.

53. The optical bio-disc according to claim 51 wherein said capture agent is a double stranded oligonucleotide sequence.

54. The optical bio-disc according to claim 51 wherein said capture agent is an antibody.

55. The optical bio-disc according to claim 51 wherein said capture agent is an antigen.

56. The optical bio-disc according to claim 51 wherein said capture agent is biotin.

57. The optical bio-disc according to claim 51 wherein said capture agent is streptavidin.

58. The optical bio-disc according to any one of claims 52, 53, 54, 55, 56, or 57 wherein said capture agent contains an amino group.

59. The optical bio-disc according to claim 58 wherein said active layer is formed from a polystyrene-co-maleic anhydride.

60. The optical bio-disc according to claim 59 wherein said amino group chemically reacts with said maleic anhydride to form a covalent bond thereby maintaining said capture agents within said target zone.

61. The optical bio-disc according to claim 60 wherein said capture agent binds with an anchor agent to thereby locate said anchor agent within said target zone.

62. The optical bio-disc according to claim 61 wherein the anchor agent is bound to one of two beads forming said dual bead complex which includes a capture bead and a reporter bead.

63. The optical bio-disc according to claim 62 wherein said anchor agent is associated with said capture bead.

64. The optical bio-disc according to claim 62 wherein said anchor agent is associated with said reporter bead.

65. The optical bio-disc according to claim 62 wherein said capture and reporter beads are linked together by a target agent to thereby form said dual bead complex.

66. The optical bio-disc according to claim 65 wherein said dual bead complex is immobilized within said target zone for inspection by an incident beam of electromagnetic radiation.

67. A method of preparing a dual bead assay for use in an optical bio-disc, said method comprising the steps of:
providing a mixture of capture beads that have transport probes covalently bound thereto;
suspending said mixture of capture beads in a hybridization solution;
adding to said mixture a target agent that hybridizes with said transport probes;
adding to said mixture reporter beads including covalently bound signal probes attached thereto;
allowing said signal probes to hybridize with said target agent to thereby form a dual bead complex including at least one capture bead and one reporter bead;
separating said dual bead complex from unbound reporter beads;
removing from said mixture said unbound reporter beads; and
loading said mixture including said dual bead complex into an optical bio-disc for analysis.

68. The method according to claim 67 wherein said step of adding said target agent is performed before said step of adding said reporter beads.

69. The method according to either claim 67 or 68 wherein said target agent is a segment of genetic material.

70. The method according to claim 69 wherein said segment of genetic material is a single strand of DNA.

71. The method according to claim 69 wherein said segment of genetic material includes a portion of double stranded DNA.

72. The method according to claim 69 wherein said segment of genetic material is a single strand of RNA.

73. The method according to claim 69 wherein said segment of genetic material includes a portion of double stranded RNA.

74. The method according to claim 67 wherein said capture beads are magnetic and said separating step is performed by use of a magnet field.

75. The method according to claim 74 wherein said magnetic field is formed by a magnet.

76. The method according to claim 74 wherein said magnetic field is formed by an electromagnet.

77. The method according to claim 67 including the further step of removing said hybridization solution for said mixture.

78. The method according to claim 77 including the further step of washing said dual bead complex to purify said mixture by further removing unbound material.

79. The method according to claim 78 including the further step of adding a buffer solution to said mixture.

80. A method of preparing a dual bead assay for use in an optical bio-disc, said method comprising the steps of:
providing a mixture of capture beads having transport probes covalently attached thereto;
suspending said mixture of capture beads in a hybridization solution;
adding to said mixture a target agent that hybridizes with said transport probes;
allowing said transport probes to hybridize with said target agent to thereby form a hybridized partial complex including at least one capture bead;
separating within said mixture said hybridized partial complex from unbound target agents;
adding to said mixture reporter beads including signal probes covalently attached thereto;
allowing said signal probes to hybridize with said target agent to thereby form a dual bead complex including at least one capture bead and one reporter bead;
separating said dual bead complex from unbound reporter beads;
removing from said mixture said unbound reporter beads; and
loading said mixture including said dual bead complex into an optical bio-disc for analysis.

81. The method according to claim 80 wherein said step of adding said target agent is performed before said step of adding said reporter beads.

82. The method according to either claim 80 or 81 wherein said target agent is a segment of genetic material.

83. The method according to claim 82 wherein said segment of genetic material is a single strand of DNA.

84. The method according to claim 82 wherein said segment of genetic material includes a portion of double stranded DNA.

85. The method according to claim 82 wherein said segment of genetic material is a single strand of RNA.

86. The method according to claim 82 wherein said segment of genetic material includes a portion of double stranded RNA.

87. The method according to claim 80 wherein said capture beads are magnetic and said separating step is performed by use of a magnet field.

88. The method according to claim 87 wherein said magnetic field is formed by a magnet.

89. The method according to claim 87 wherein said magnetic field is formed by an electromagnet.

90. The method according to claim 80 including the further step of removing said hybridization solution for said mixture.

91. The method according to claim 90 including the further step of washing said dual bead complex to purify said mixture by further removing unbound material.

92. The method according to claim 91 including the further step of adding a buffer solution to said mixture.

93. A method of testing for the presence of a target-DNA in a DNA sample by use of an optical bio-disc, said method comprising the steps of:
preparing a DNA sample to be tested for the presence of a target-DNA;
preparing a plurality of reporter beads each having covalently attached thereto a plurality of strands of signal-DNA and an anchor agent, the target-DNA and the signal-DNA being complementary;
preparing a plurality of capture beads each having covalently attached thereto a plurality of transport-DNA, the target-DNA and transport-DNA being complimentary;
mixing said DNA sample, said plurality of reporter beads, and said plurality of capture beads to thereby form a test sample, the transport-DNA and the signal-DNA being non-complimentary;
allowing hybridization between said signal-DNA, any target-DNA, and transport-DNA existing in the DNA sample to thereby form a dual bead complex including at least one capture bead and one reporter bead;
removing from the test sample reporter beads and capture beads that are not associated with the dual bead complex;
depositing said test sample in a flow channel of an optical bio-disc which is in fluid communication with a target zone, the target zone including a plurality of capture agents each including an amino group that attaches to an active layer to immobilize the capture agents within the target zone;
allowing any anchor agent to bind with the capture agents so that reporter beads associated with the dual bead complex are maintained within the target zone; and
detecting any dual bead complexes in the target zone to thereby determine whether target-DNA is present in the DNA sample.

94. A method of testing for the presence of a target-DNA in a test sample by use of an optical bio-disc, said method comprising the steps of:
preparing a test sample to be tested for the presence of a target-DNA;
preparing a plurality of reporter beads each having covalently attached thereto a plurality of strands of signal-DNA, the target-DNA and the signal-DNA being complementary;
preparing a plurality of capture beads each having covalently attached thereto a plurality of transport-DNA and an anchor agent, the target-DNA and transport-DNA being complimentary;
depositing a plurality of capture beads and reporter beads in a mixing chamber, each of said reporter beads and said capture beads including signal-DNA and transport-DNA, respectively, being non-complimentary to each other;
depositing said test sample in the mixing chamber of an optical bio-disc which is linked to a target zone by a connecting flow channel allowing any target-DNA existing in the test sample to bind to the signal-DNA and the transport-DNA on the reporter and the capture bead, respectively, to thereby form a dual bead complex;
rotating the optical bio-disc to cause the dual bead complex to move from the mixing chamber through the flow channel and into the target zone, the target zone including a plurality of capture agents each including an amino group that attaches to an active layer to immobilize the capture agents within the target zone, said capture agent having affinity for the anchor agent;
allowing any anchor agent to bind with the capture agent so that capture beads associated with dual bead complex are maintained within the capture zone;
removing from the target zone reporter beads that are free of any dual bead complex; and
detecting any dual bead complex in the target zone to thereby determine whether target-DNA is present in the test sample.

95. A method of testing for the presence of a target-RNA in a test sample by use of an optical bio-disc, said method comprising the steps of:
preparing a test sample to be tested for the presence of a target-RNA;
preparing a plurality of reporter beads each having covalently attached thereto a plurality of strands of signal-DNA, the target-RNA and the signal-DNA being complementary;
preparing a plurality of capture beads each having covalently attached thereto a plurality of transport-DNA and an anchor agent, the target-RNA and transport-DNA being complimentary;
depositing a plurality of capture beads and reporter beads in a mixing chamber, each of said reporter beads and capture beads including the signal-DNA and the transport-DNA, respectively, being non-complimentary to each other;
depositing said test sample in the mixing chamber of an optical bio-disc which is linked to a target zone by a connecting flow channel allowing any target-RNA existing in the test sample to hybridize with the signal-DNA and the transport-DNA on the reporter and the capture bead, respectively, to thereby form a dual bead complex;
rotating the optical bio-disc to cause the dual bead complex to move from the mixing chamber through the flow channel and into the target zone, the target zone including a plurality of capture agents each including an amino group that attaches to an active layer to immobilize the capture agents within the target zone, said capture agent and said anchor agent having affinity to each other;
allowing any anchor agent to bind with the capture agent so that capture beads associated with dual bead complex are maintained within the capture zone;
removing from the target zone reporter beads that are free of any dual bead complex; and
detecting any dual bead complex in the target zone to thereby determine whether target-RNA is present in the test sample.

96. A method of testing for the presence of a target-antigen in a test sample by use of an optical bio-disc, said method comprising the steps of:
preparing a test sample to be tested for the presence of a target-antigen;
preparing a plurality of reporter beads each having covalently attached thereto a plurality of signal-antibody, the signal-antibody having an affinity to epitopes on the target-antigen;
preparing a plurality of capture beads each having covalently attached thereto a plurality of transport-antibody and an anchor agent, the transport-antibody having affinity to epitopes on the target-antigen;
depositing the capture beads and the reporter beads in a mixing chamber, each of said reporter beads and capture beads including the signal-antibody and the transport-antibody, respectively, having no affinity to each other;
depositing said test sample in the mixing chamber of an optical bio-disc which is linked to a target zone by a connecting flow channel allowing any target-antigen existing in the test sample to bind to the signal-antibody and the transport-antibody on the reporter and the capture bead, respectively, to thereby form a dual bead complex;
rotating the optical bio-disc to cause the dual bead complex to move from the mixing chamber through the flow channel and into the target zone, the target zone including a plurality of capture agents each including an amino group that attaches to an active layer to immobilize the capture agents within the target zone;
allowing any anchor agent to bind with the capture agent so that capture beads associated with dual bead complex are maintained within the capture zone;
removing from the target zone reporter beads that are free of any dual bead complex; and
detecting any dual bead complex in the target zone to thereby determine whether target-antigen is present in the test sample.

97. The method according to any one of claims 93, 94, 95, or 96 wherein the said dual bead complex is detected by directing a beam of electromagnetic energy from a disc drive assembly toward said target zone and analyzing electromagnetic energy returned from said target zones.

98. A method of making an optical bio-disc for testing for the presence of a target-DNA in a DNA sample, said method comprising the steps of:
providing a substrate having a center and an outer edge;
encoding information on an information layer associated with the substrate, said encoded information being readable by a disc drive assembly to control rotation of the disc;
forming a target zone in association with said substrate, said target zone disposed at a predetermined location relative to said center of said substrate;
applying an active layer in said target zone;
depositing within said target zone, a plurality of strands of capture-DNA each including an amino group that covalently attaches to said active layer to immobilize said strands of capture-DNA within said target zone;
forming a flow channel in fluid communication with said target zone;
forming a mixing chamber in fluid communication with the flow channel;
depositing a plurality of reporter beads in the mixing chamber, each of said reporters including a signal-DNA that has an affinity for the target-DNA;
depositing a plurality of capture beads in the mixing chamber, each of said capture bead including a transport-DNA that hybridizes with a portion of the target-DNA and is complementary to said capture-DNA, the transport-DNA and signal-DNA being non-complimentary; and
designating an input site associated with the mixing chamber, the input site implemented to receive a DNA sample to be tested for the presence of any target-DNA, so that when the DNA sample is deposited in the mixing chamber hybridization occurs between the signal-DNA, the target-DNA, and the transport-DNA to thereby form a dual bead complex including at least one reporter bead and one capture bead, so that when the disc is rotated, the dual bead complex move into the target zone and hybridization occurs between the anchor-DNA and the capture-DNA to thereby place the dual bead complex in the target zone.

99. A method of making an optical bio-disc for determining the presence of a target-DNA in a test sample, said method comprising the steps of:
providing a substrate having a center and an outer edge;
encoding information on an information layer associated with the substrate, the encoded information being readable by a disc drive assembly to control rotation of the disc;
forming a target zone in association with the substrate, the target zone disposed at a predetermined location relative to the center of the substrate;
applying an active layer in the target zone;
depositing within the target zone, a plurality of strands of capture-DNA each including an amino group that covalently attaches to the active layer to immobilize the strands of capture-DNA within the target zone; and
forming a flow channel in fluid communication with the target zone.

100. The method according to claim 99 wherein the flow channel is implemented to receive a test sample including sample-DNA, a plurality of reporter beads each having covalently attached thereto a plurality of strands of signal-DNA, and a plurality of capture beads each having covalently attached thereto a plurality of strands of transport-DNA.

101. The method according to claim 100 wherein the capture-DNA and the signal-DNA are non-complementary, the transport-DNA and the capture-DNA are complimentary.

102. The method according to claim 101 wherein the sample-DNA to be tested for the presence of a target-DNA is complementary to the transport-DNA and the signal-DNA so that when the test sample is deposited in the flow channel, a dual bead complex including at least one reporter bead and one capture bead is formed, and when the disc is rotated the dual bead complex moves into the target zone and hybridization occurs between any transport-DNA and the capture-DNA thereby maintaining capture beads and dual bead complexes within the target zone.

103. A method of making an optical bio-disc for determining the presence of a target-antigen in a test sample, said method comprising the steps of:
providing a substrate having a center;
encoding information on an information layer associated with the substrate, the encoded information being readable by a disc drive assembly to control rotation of the disc;
forming a target zone in association with the substrate, the target zone disposed at a predetermined location relative to the center of the substrate;
depositing an active layer in the target zone;
depositing in the target zone, a plurality of capture agents each including an amino group that covalently attaches to the active layer to immobilize the capture agents within the target zone; and
forming a flow channel in fluid communication with the target zone, the flow channel implemented to receive a test sample including target-antigen.

104. A method of using the disc made according to claim 103 wherein a plurality of reporter beads each having covalently attached thereto a plurality of signal-antibody, and a plurality of capture beads each having covalently attached thereto a plurality of transport-antibody are introduced into the flow channel.

105. The method according to claim 104 wherein the signal-antibody has no affinity for the capture agent, the transport-antibody has affinity for the capture agent, and the transport-antibody and the signal-antibody have affinity to different epitopes on the target-antigen so that when the test sample is deposited in the flow channel, a dual bead complex including at least one reporter bead and one capture bead is formed.

106. The method according to claim 105 wherein when the disc is rotated, the dual bead complex moves into the target zone and binding occurs between any transport-antibody and the capture agent to thereby maintain capture beads and dual bead complexes within the target zone.

107. A method of making an optical bio-disc to test for the presence of a target agent in a test sample, the method comprising the steps of:
providing a substrate having a center and an outer edge;
encoding information on an information layer associated with the substrate, the encoded information being readable by a disc drive assembly to control rotation of the disc;
forming a target zone in association with the substrate, the target zone disposed at a predetermined location relative to the center of the substrate;
depositing an active layer in the target zone;
depositing a plurality of capture agents in the target zone, each capture agent including an amino group that covalently attaches to the active layer to immobilize the capture agent within the target zone;
forming a flow channel in fluid communication with the target zone;
forming a mixing chamber in fluid communication with the flow channel;
depositing a plurality of reporter beads in the mixing chamber, each of the reporter beads having covalently attached thereto a plurality of signal probes, each of the signal probe having affinity to the target agent; and
depositing a plurality of capture beads in the mixing chamber, each of the capture beads having covalently attached thereto a plurality of transport probes and an anchor agent, each of the transport probe having affinity to the target agent, the transport probes and signal probes having no affinity toward each other, and the capture agents and the anchor agents having specific affinity to each other.

108. The method according to any one of claims 93, 94, 95, or 96 wherein the said dual bead complex is detected by directing a beam of electromagnetic energy from a disc drive assembly toward said target zone and analyzing electromagnetic energy returned from said target zones.

109. An optical bio-disc, comprising:
a substrate having a center and an outer edge, said substrate forming a distal layer of the bio-disc, said substrate having a top surface and a bottom surface relative to an interrogation beam of electromagnetic energy directed from a disc drive;
a reflective layer formed on the bottom surface of said substrate;
an active layer associated with said substrate and said reflective layer; and
a strand of capture DNA including an amino group which has an affinity for said active layer so that said amino group covalently attaches to said active layer to immobilize said strand of DNA in a target zone disposed between said center and said outer edge.

110. The optical bio-disc according to claim 109 wherein said strand of capture DNA is complementary to a strand of anchor DNA which includes a dual bead complex with at least a reporter bead and a capture bead that is detectable by said interrogation beam.

111. The optical bio-disc according to either claim 109 or 110 wherein said strand of capture DNA is a single strand of DNA.

112. The optical bio-disc according to either claim 109 or 110 wherein said strand of capture DNA includes a double strand of DNA.

113. The optical bio-disc according to either claim 109 or 110 wherein said active layer is formed from a modified polystyrene.

114. The optical bio-disc according to either claim 109 or 110 wherein said reflective layer is interposed between said substrate and said active layer.

115. The method according to any of the claims 98, 99, 103, or 107 wherein said removing step is performed by rotating the optical bio-disc.
